(19) United States
(12) Patent Application Publication
(10) Pub. No.: US 2023/0129710 A1
(43) Pub. Date: Apr. 27, 2023

(54) Methods and Reagents for Synthesizing Nucleosides and Analogues Thereof

(71) Applicants: Simon Fraser University, Burnaby (CA); Merck, Sharp & Dohme Corp., Rahway, NJ (US)

(72) Inventors: Robert Britton, North Vancouver (CA); Adluri Bharanishashank, Saint Laurent (CA); Michael Meanwell, Nanaimo (CA); Steven Silverman, Jersey City, NJ (US)

(21) Appl. No.: 17/913,920

(22) PCT Filed: Mar. 25, 2021
(86) PCT No.: PCT/IB2021/052464

§ 371 Date: Sep. 23, 2022

Related U.S. Application Data
(60) Provisional application No. 62/994,349, filed on Mar. 25, 2020.

(51) Int. Cl. C07H 1/00; C07H 19/067

(52) U.S. Cl. CPC C07H 1/00; C07H 19/067

(57) ABSTRACT

The present invention relates to methods and intermediates for the synthesis of nucleosides and nucleoside analogues (NAs). More specifically, the present invention relates to methods of synthesizing nucleosides and NAs, using simple achiral materials by a ‘one-pot’ proline-catalyzed halogenation of a heteroaryl-substituted acetaldehyde together with a tandem enantioselective aldol reaction followed by a reduction or organometallic addition and cyclization (annulation) reaction involving halide displacement.

### FIELD

The present invention relates to synthesis of nucleosides and analogues thereof. More specifically, the present invention relates to methods and reagents for the synthesis of nucleosides and analogues thereof.

### BACKGROUND

Nucleosides play key roles in diverse cellular processes ranging from cell signalling to metabolism (1). The prebiotic synthesis of DNA (25) and RNA (26) is proposed to involve couplings between nucleobase-type enamines and glyceraldehyde to form a nucleobase iminium ion prior to the furanose in a “ribose-last” approach.

Synthetic nucleoside analogues (NAs), designed to mimic their natural counterparts, are widely exploited in medicinal chemistry and used as tool compounds in chemical biology (2-18). NAs have been used in the treatment of cancer (2, 6) and represent the largest class of small molecule antivirals (3, 4). Mechanistically, NAs can operate as toxic antimetabolites that interfere with nucleic acid synthesis (4). Alternatively, following in vivo phosphorylation, the resulting nucleotide analogues can inhibit enzymes involved in cancer cell growth or viral replication (e.g., DNA/RNA polymerases, ribonucleotide reductases or nucleoside phosphorylases) (2, 4). NAs have also demonstrated promise as epigenetic modulators, and both decitabine and azacitidine inhibit DNA methyltransferase and have been approved for cancer therapy (4).

The processes for synthesis of NAs, however, are often protracted, not amenable to diversification and rely on a limited pool of chiral carbohydrate starting materials and therefore present many challenges (e.g., 19-24, 27, 33, 42-44).

Locked nucleic acids (LNAs) (39) are conformationally restricted NAs that demonstrate improved stability and their incorporation in antisense oligonucleotides can lead to significant increases in specificity and potency. However, much like syntheses of other C4′-modified NAs, the synthesis of LNAs is often protracted.

### SUMMARY

The present invention relates to synthesis of nucleosides and analogues thereof.

In one aspect, the present invention provides a method of synthesizing a nucleoside or analogue thereof by: halogenating an aryl- or heteroaryl-substituted acetaldehyde compound by proline catalysis followed by an enantioselective aldol reaction to yield a halohydrin compound; reducing the halohydrin compound to yield a halohydrin diol compound; and contacting the halohydrin diol compound with a Lewis acid or a base in an annulative halide displacement (AHD) reaction, to yield a nucleoside or analogue thereof.

In some embodiments, the Lewis acid may be InCl₃ or Sc(OTf)₃.

In some embodiments, the halohydrin diol compound may be separated prior to treatment with the Lewis base.

In some embodiments, the base may be NaOH.

In some embodiments, the base-AHD reaction may yield a C3′,C5′-protected nucleoside or analogue thereof.

In alternative aspects, the present invention provides a method of preparing an intermediate in the synthesis of a nucleoside or analogue thereof by: halogenating a heteroaryl-substituted acetaldehyde compound by proline catalysis followed by an enantioselective aldol reaction to yield an halohydrin compound; and reducing the halohydrin compound to obtain a halohydrin diol compound, to yield an intermediate in the synthesis of a nucleoside or analogue thereof.

In alternative aspects, the present invention provides a method of synthesizing a nucleoside or analogue thereof by: (i) providing a halohydrin diol compound; and ii) contacting the halohydrin diol compound with a Lewis acid or a base in an annulative halide displacement (AHD) reaction, to yield a nucleoside or analogue thereof.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 is a schematic showing the synthesis of nucleosides and nucleoside analogues (NAs) through a short sequence of reactions involving an asymmetric α-fluorination aldol reaction (αFAR) followed by a cyclization (annulation) reaction involving fluoride displacement (AFD reaction). Het=heteroaryl.

FIGS. 2A-C show the synthesis of the pyrazolyl NA 17. A: The prebiotic synthesis of nucleosides is proposed to involve the coupling of nucleoside enamines such as 12 with glyceraldehyde in a “ribose-last” approach. A synthetic, ribose-last approach to NAs involves an aldol reaction of the iminium ion surrogate 14. B: Examination of a proline catalyzed α-fluorination and aldol reaction revealed this process is compatible with α-pyrazolyl aldehyde 15, providing the fluorohydrins 16 in good yield and enantioselectivity. Reduction and an annulative fluoride displacement (AFD) provides a rapid route to NA 17. C: Mechanistic studies reveal that the AFD proceeds via stereochemical inversion (S_{N}2 reaction) followed by epimerization. NFSI=N-fluorobenzenesulfonimide; DMF=dimethylformamide; MeCN=acetonitrile; OTf=triflate.

FIGS. 3A-F show nucleoside and NA synthesis. A: A 4-step reaction sequence converts readily available starting materials into enantioenriched and naturally configured p-D-NAs. B: AFD to produce uracil, thymine, pyrazolyl and 5-pyrimidinyl nucleosides and NAs can be promoted by NaOH. C: AFD to produce trifluoromethyl uracil, triazolyl, phthalimidyl, deazaadenine, and adenosine nucleosides and NAs can be promoted by the Lewis acids Sc(OTf)₃ or InCl₃. D: NAs protected at both the C3′ and C5′-alcohol functions. E: Non-natural nucleosides (L-enantiomers) using D-proline to catalyze the αFAR reaction F: C2′-modified NAs. ^{a} TEMPO, BAIB, dioxane (92% from 34). ^{b} i) thiocarbonyldiimidazole, THF; ii) Bu₃SnH, azobisisobutyronitrile (55% over 2 steps from 35). ° i) TEMPO, BAIB, dioxane; ii) MeMgBr, THF, −78° C. (80% over 2 steps from 34). ^{d} DAST, CH₂Cl₂ then HCl, MeOH (53% from 35). TEMPO=2,2,6,6-Tetramethylpiperidin-1-yl)oxyl; BAIB=bis(acetoxy)iodobenzene; THF=tetrahydrofuran; DAST=diethylaminosulfur trifluoride.

FIGS. 4A-E show the rapid synthesis of C4′-modified and other NAs. A: The addition of organomagnesium reagents to αFAR products generates tertiary alcohols that undergo direct AFD or Lewis acid/base-promoted AFD to C4′-modified NAs. B: The large-scale (˜380 g) production of fluorohydrin 55 supports the synthesis of MK-3682 (HCV RNA polymerase inhibitor). C: Reductive amination of fluorohydrin 59 provides a direct route to iminonucleoside 60. D: Preparation of C4′-modified C2′-deoxy NA 62 by exploiting the inherent protection of the C3′ and C5′-OH functions. E: Synthesis of two LNAs 65 and 68. ^{a}Yield from keto-fluorohydrin aldol adduct.^{b} Combined yield of diastereomers. ^{c} Product following heating of crude reaction mixture to 50° C. with CSA and dimethoxyacetone. ^{d}Product following treatment of crude reaction mixture with aqueous HCl. ^{e} Starting from a single fluorohydrin 59.

### DETAILED DESCRIPTION

The present disclosure provides, in part, methods and intermediates for the synthesis of nucleosides or analogues thereof.

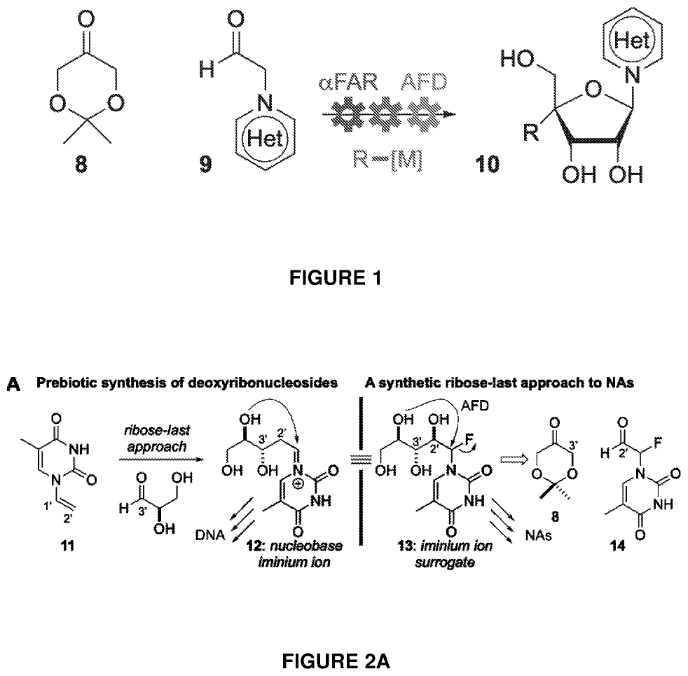

FIG. 1 shows a proline catalyzed α-fluorination and aldol reaction (α-FAR) and annulative fluoride displacement (AFD) for nucleoside analogue (NA) synthesis using simple achiral building blocks. The synthesis includes a one-pot, proline-catalyzed α-fluorination-aldol reaction of heteroaryl-substituted acetaldehydes 9 followed by reduction or organometallic addition and AFD. This process allows, for example, direct access to C3′/C5′ protected NAs 10 (and C2′ modified NAs), provides flexibility in nucleobase substitution, offers a direct route to C4′ modified NAs, etc.

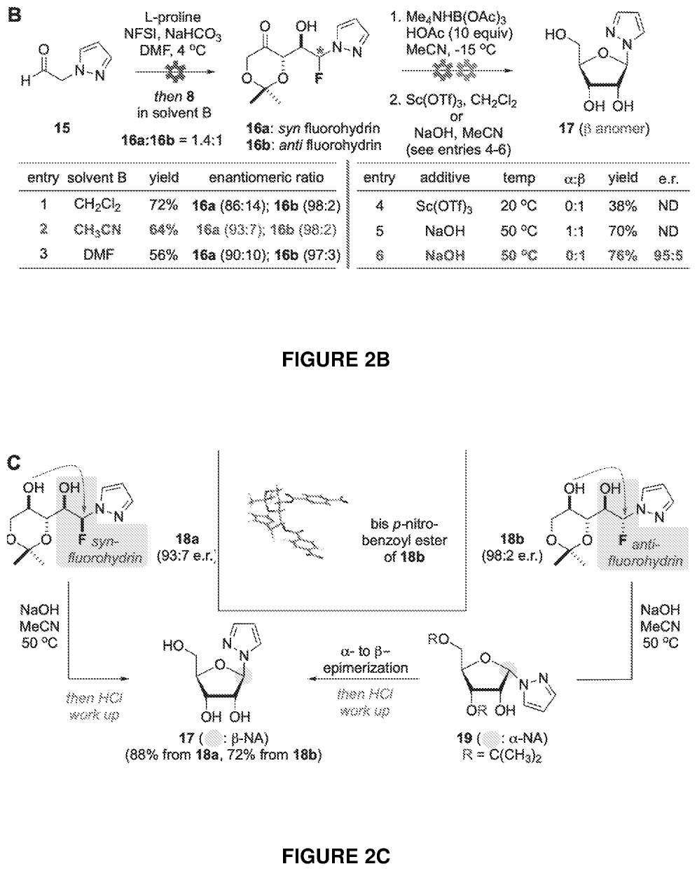

In some embodiments, the methods include a complementary (ribose-last) approach, that also involves the terminal cyclization of a nucleobase-iminium ion, for the synthesis of nucleosides and NAs. In a proposed prebiotic synthesis of DNA, couplings between nucleobase-type enamines 11 (FIG. 2A) and glyceraldehyde form a nucleobase iminium ion 12 prior to the furanose in a “ribose-last” approach. As a synthetic equivalent to a nucleobase iminium ion 12, the halogenated acyclic NA 13 (FIG. 2A) was proposed. Without being bound to any particular theory, formation of the ribonucleoside C2′-C3′ bond and control of both the relative and absolute stereochemistry would be possible through an organocatalytic aldol reaction of a dihydroxyacetone derivative (e.g., 8)(30) and the α-haloaldehyde 14 (FIG. 2A). Accordingly, methods described herein include i) harnessing the reactivity of α-haloaldehydes (e.g., 28, 29, 31, 32, 35), which are known to be unstable, coupled with a nucleobase connected at the same position (e.g., 8), and ii) the development of an annulative halide displacement (AHD) reaction to form the ribose ring in the last step.

In some embodiments, the present disclosure provides a method of synthesizing nucleosides and NAs, using simple achiral materials, through a short (2-3 step) sequence of reactions involving a ‘one-pot’ proline-catalyzed α-halogenation of a heteroaryl-substituted acetaldehyde together with a tandem enantioselective aldol reaction (αHAR) followed by a reduction or organometallic addition and cyclization (annulation) reaction involving halide displacement (AHD).

More specifically, in some embodiments, the present disclosure provides a method of synthesizing a nucleoside or analogue thereof, by:

(i) halogenating an aryl- or heteroaryl substituted acetaldehyde compound by proline catalysis to yield an α-haloaldehyde compound that is then coupled by proline catalysis with a ketone to produce a halohydrin compound;

ii) reducing an halohydrin compound to yield a halohydrin diol compound; and

iii) contacting the halohydrin diol compound with a Lewis acid or a base in an annulative halide displacement (AHD) reaction,

to yield a nucleoside or analogue thereof.

In some embodiments, the Lewis acid may be, without limitation, a halophilic Lewis acid.

In some embodiments, the Lewis acid may be, without limitation, InCl₃ or Sc(OTf)₃.

In some embodiments, Lewis acid-promoted AHD may yield a C2′,C3′-protected nucleoside or NA.

In some embodiments, Lewis acid-promoted AHD may result in protecting group migration, i.e., may yield a NA with a migrated acetonide protecting group.

In some embodiments, Lewis acid-promoted AHD may result in deprotection.

In some embodiments, the base may be NaOH.

In some embodiments, the base-promoted AHD may yield a C3′,C5′-protected NA.

In some embodiments, the αHAR reaction products may be reduced and separated prior to treatment with a Lewis base.

In some embodiments, the present disclosure provides a method of preparing an intermediate in the synthesis of a nucleoside or analogue thereof, by:

(i) halogenating a heteroaryl-substituted acetaldehyde compound by proline catalysis followed by an enantioselective aldol reaction to yield a halohydrin compound;

ii) reducing the halohydrin compound to obtain a halohydrin diol compound,

to yield an intermediate in the synthesis of a nucleoside or analogue thereof.

In some embodiments, the present disclosure provides a method of synthesizing a nucleoside or analogue thereof, by:

(i) providing a halohydrin diol compound; and

ii) contacting the halohydrin diol compound with a Lewis acid or a base in an annulative halide displacement (AHD) reaction,

to yield a nucleoside or analogue thereof.

By “halohydrin” is meant a compound containing a functional group in which a halogen and a hydroxyl are bonded to adjacent groups. A halohydrin can have the following the general structure, where R¹ and R² may be any suitable group, as indicated herein, and X may be as indicated herein:

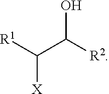

In some embodiments, the halohydrin compound may have the following general structure, where NB and X may be as indicated herein:

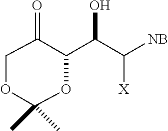

In some embodiments, the halohydrin compound may be functionalized with an aryl or heteroaryl i.e., NB may be an aryl or heteroaryl.

In some embodiments, the halohydrin diol compound may have the following general structure, where NB and X may be as indicated herein:

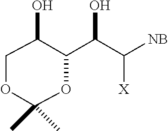

In some embodiments, the halohydrin diol compound may be functionalized with an aryl or heteroaryl i.e., NB may be an aryl or heteroaryl.

In some embodiments, the present disclosure provides the following nucleosides or analogues thereof, including without limitation diastereomers thereof, where NB may be as indicated herein and each R may independently be —OH, —OC(CH₃)₂O—, —(CH₂)₃—, —CH₂SCH₂—, or —CH₂OCH₂—;

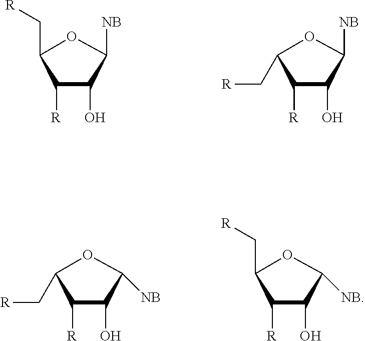

In some embodiments, the present disclosure provides the following compounds, or enantiomers thereof, where NB and X may be as indicated herein, and each R may independently be —OH, —OC(CH₃)₂O—, —(CH₂)₃—, —CH₂SCH₂—, or —CH₂OCH₂—, for use as an intermediate in the synthesis of a nucleoside or analogue thereof:

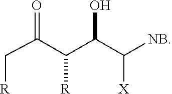

In some embodiments, the present disclosure provides the following compounds, or enantiomers thereof, where NB and X may be as indicated herein, Y may be CH₂, O, S, NR, where R may be alkyl or aryl, and Z may be a protecting group for an alcohol, including without limitation, acetonide, silyl protecting group, alkyl protecting group or aryl protecting group (including cyclic or acyclic), for use as an intermediate in the synthesis of a nucleoside or analogue thereof:

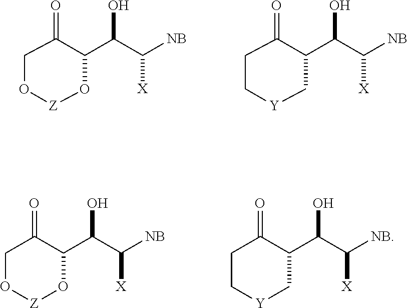

In some embodiments, the present disclosure provides the following compounds, or enantiomers thereof, where NB and X may be as indicated herein, for use as an intermediate in the synthesis of a nucleoside or analogue thereof:

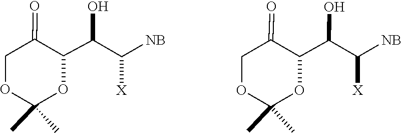

In some embodiments, the present disclosure provides the following compounds, or enantiomers thereof, where NB and X may be as indicated herein, and Y may be CH₂, O, S, NR, where R may be alkyl or aryl, for use as an intermediate in the synthesis of a nucleoside or analogue thereof:

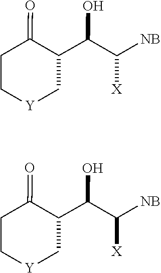

In some embodiments, the methods disclosed herein provide rapid access to intermediates in the synthesis of nucleosides or analogues thereof in good enantioselectivity and/or yield, for example, greater than about 10 g to about 400 g, or any value in between, for example 10 g, 15 g, 20 g, 25 g, 50 g, 75 g, 100 g, 125 g, 150 g, 200 g, 250 g, 300 g, 350 g, or 400 g. Accordingly, the methods disclosed herein may be used in the process scale production of nucleosides and/or NAs.

In some embodiments, the methods disclosed herein enable direct access to C3′/C5′ protected NA 3, where R may be alkyl, alkynyl or aryl and NB may be as indicated herein (and hence C2′ modified NAs), provide flexibility in nucleobase substitution, and/or offer a direct route to C4′ modified NAs:

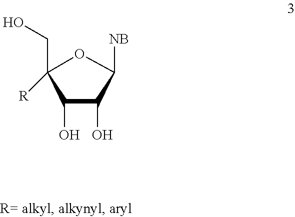

In some embodiments, in the methods disclosed herein, carbonyl reduction followed by an annulative halide displacement affords naturally configured p-D-NAs with both the C3′—OH and C5′-OH functions protected.

In some embodiments, the methods disclosed herein enable direct incorporation of a wide range of nucleobases and the selective functionalization of the C2′ position of the furanose core of natural nucleosides and NAs including, without limitation, C-linked or L-configured NAs.

In some embodiments, in the methods disclosed herein, replacement of the reductant with an organomagnesium reagent provides direct access to an array of C4′-modified NAs including, without limitation, locked nucleic acids (LNAs).

In some embodiments, the synthesis methods disclosed herein may be useful, without limitation, in the production of D- and L-nucleosides and nucleoside analogues, locked nucleic acids, iminonucleosides, C4′-modified nucleosides and/or C2′-modified nucleosides.

In some embodiments, the methods disclosed herein may be useful as a tool for drug design.

In some embodiments, the methods disclosed herein may be useful in the preparation of diversity libraries. For example, larger collections of C4′-modified NAs (e.g., focused screening libraries) can be generated using the methods described herein.

By “nucleoside” is meant a glycosylamine having a nitrogenous base or “nucleobase” or “NB” and a sugar ring (e.g., ribose or deoxyribose), in which the anomeric carbon is linked through a glycosidic bond to the N9 of a purine (e.g., adenine or guanine) or the N1 of a pyrimidine (e.g., cytosine, thymine, or uracil). Nucleosides include both L- and D-nucleoside isomers. Examples of nucleosides include cytidine, uridine, adenosine, guanosine, thymidine and inosine.

Nucleoside analogues (NAs) are compounds that are structurally similar to naturally occurring nucleosides. NAs may include, without limitation, compounds with modifications at positions C1′, C2′, C3′, C4′ and/or C5′ of the sugar ring. In some embodiments, NAs may exist as a free triol or may be phosphorylated at C3′ and/or C5′. In some embodiments, NAs may include, without limitation, compounds with a saturated or unsaturated carbocyclic ring.

In some embodiments, NAs may include nitrogen in the sugar ring, for example as a replacement for the naturally occurring oxygen, and/or may include N—R groups, where R may be without limitation alkyl, allyl, alkynyl or benzyl. In some embodiments, NAs that include sulphur in the sugar ring, for example as a replacement for the naturally occurring oxygen, are specifically excluded.

The “NB” or nucleobase of NAs may be any aryl or heteroaryl attached from the C1 position to a carbon or nitrogen atom. NBs may also be modified, for example, may be 5,6-dihydrouracil, 5-methylcytosine, 5-hydroxymethylcytosine, 5,5,5-trifluoromethylthymine, 5-fluorouracil, 2-thiouracil, 4-methylbenzimidazole, hypoxanthine, 7-deazaguanine, 7-deazaadenine, indole, imidazole, triazole, pyrrole, pyrazole, etc. It is to be understood that enantiomers of aldol products (halohydrins) can be produced using D-proline catalysis and may be used to prepare enantiomeric NAs.

By “aryl” is meant a monocyclic or bicyclic aromatic ring containing only carbon atoms, including for example, 5-14 members, such as 5, 6, 7, 8, 9, 10, 11, 12, 13, or 14 members. Examples of aryl groups include phenyl, biphenyl, naphthyl, indanyl, indenyl, tetrahydronaphthyl, 2,3-dihydrobenzofuranyl, dihydrobenzopyranyl, 1,4-benzodioxanyl, and the like. Unless stated otherwise specifically herein, the term “aryl” is meant to include aryl groups optionally substituted by one or more substituents as described herein.

“Heteroaryl” refers to a single or fused aromatic ring group containing one or more heteroatoms in the ring, for example N, O, S, including for example, 5-14 members, such as 5, 6, 7, 8, 9, 10, 11, 12, 13, or 14 members. Examples of heteroaryl groups include furan, thiophene, pyrrole, oxazole, thiazole, imidazole, pyrazole, isoxazole, isothiazole, 1,2,3-oxadiazole, triazole (e.g., 1,2,3-triazole or 1,2,4-triazole), 1,3,4-thiadiazole, tetrazole, pyrazole, pyridine, pyridazine, pyrimidine, 2,6-dichloropyrimidine pyrazine, 1,3,5-triazine, imidazole, benzimidazole, benzoxazole, benzothiazole, indolizine, indole, isoindole, benzofuran, benzothiophene, 1H-indazole, purine, 4H-quinolizine, quinoline, isoquinoline, cinnoline, phthalazine, quinazoline, quinoxaline, 1,8-naphthyridine, pteridine, uracil, thymine, deazadenine, phthalimide, adenine, and the like. Unless stated otherwise specifically herein, the term “heteroaryl” is meant to include heteroaryl groups optionally substituted by one or more substituents as described herein.

Halogens include bromine, chlorine, fluorine, iodine, etc. and are represented by “X” in the chemical structures disclosed herein. In some embodiments, a halogen may include chlorine or fluorine. According, “halo” refers to bromo, chloro, fluoro, iodo, etc. A halide is a halogen atom bearing a negative charge. By “halogenating” is meant introducing a halogen atom into a compound or molecule.

“Optional” or “optionally” means that the subsequently described event of circumstances may or may not occur, and that the description includes instances where the event or circumstance occurs one or more times and instances in which it does not. For example, “optionally substituted alkyl” means that the alkyl group may or may not be substituted and that the description includes both substituted alkyl groups and alkyl groups having no substitution, and that the alkyl groups may be substituted one or more times.

Examples of optionally substituted alkyl groups include, without limitation, methyl, ethyl, propyl, butyl, pentyl, hexyl, isopropyl, isobutyl, sec-butyl, tert-butyl, etc. Examples of suitable optional substituents include, without limitation, H, F, Cl, CH₃, OH, OCH₃, CF₃, CHF₂, CH₂F, CN, halo, and C₁₋₁₀ alkoxy.

As used herein the singular forms “a”, “and”, and “the” include plural referents unless the context clearly dictates otherwise. For example, “a compound” refers to one or more of such compounds. Throughout this application, it is contemplated that the term “compound” or “compounds” refers to the compounds discussed herein and includes precursors and derivatives of the compounds. The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Any formulas, structures or names of compounds described in this specification that do not specify a particular stereochemistry are meant to encompass any and all existing isomers as described above and mixtures thereof in any proportion. When stereochemistry is specified, the invention is meant to encompass that particular isomer in pure form or as part of a mixture with other isomers in any proportion. Single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. These procedures can be repeated, if desired, to increase the enantiomeric purity of a compound. When the compounds described herein contain olefmic double bonds or other centers of geometric asymmetry, and unless otherwise specified, it is intended that the compounds include the cis, trans, Z- and E-configurations. Likewise, all tautomeric forms are also intended to be included.

The starting materials can be obtained from commercial sources, prepared from commercially available organic compounds, prepared using known synthetic methods.

The present invention will be further illustrated in the following examples.

### Examples

Materials and Methods

General Considerations

L- and D-proline (99% purity) were purchased from Alfa Aesar. All reactions described were performed at ambient temperature and atmosphere unless otherwise specified. Column chromatography was carried out with 230-400 mesh silica gel (E. Merck, Silica Gel 60). Concentration and removal of trace solvents was done via a Buchi rotary evaporator using acetone-dry-ice condenser and a Welch vacuum pump.

Nuclear magnetic resonance (NMR) spectra were recorded using deuterochloroform (CDCl₃), deuteromethanol (CD₃OD), deuteroacetone ((CD₃)₂CO), deuteroacetonitrile (CD₃CN) or deuterodimethyl sulfoxide (DMSO-d₆) as the solvent. Signal positions (δ) are given in parts per million from tetramethylsilane (δ 0) and were measured relative to the signal of the solvent (¹H NMR: CDCl₃: δ 7.26; CD₃OD: δ 3.31; (CD₃)₂CO: δ 2.05; CD₃CN: δ 1.96; DMSO-d₆: δ 2.50; ¹³C NMR: CDCl₃: δ 77.16; CD₃OD: δ 49.00; (CD₃)₂CO: δ 29.84; CD₃CN: δ 1.32; DMSO-d₆: 39.5). Coupling constants (J values) are given in Hertz (Hz) and are reported to the nearest 0.1 Hz. ¹H NMR spectral data are tabulated in the order: multiplicity (s, singlet; d, doublet; t, triplet; q, quartet; sept, septet; m, multiplet; brbroad), coupling constants, number of protons. NMR spectra were recorded on a Bruker Avance 600 equipped with a QNP or TCI cryoprobe (600 MHz), Bruker 400 (400 MHz) or Bruker 500 (500 MHz). Diastereomeric ratios (dr) are based on analysis of crude ¹H NMR. Assignments of ¹H are based on analysis of ¹H-¹H-COSY and nOe spectra. Assignments of ¹³C are based on analysis of HSQC spectra.

High performance liquid chromatography (HPLC) analysis was performed on an Agilent 1100 HPLC, equipped with a variable wavelength UV-Vis detector.

Infrared (IR) spectra were recorded neat on a Perkin Elmer Spectrum Two FTIR spectrometer. Only selected, characteristic absorption data are provided for each compound.

Optical rotation was measured on a Perkin-Elmer Polarimeter 341 at 589 nm.

General Procedures

General Procedure A (one-pot organocatalytic α-fluorination/aldol reaction)

A sample of aldehyde (1.5 equiv.) was added to a stirred suspension of NFSI (1.5 equiv.), L-proline (1.5 equiv.), and NaHCO₃ (1.5 equiv.) in DMF (0.75 M) at 4° C. When complete conversion to the α-fluoroaldehyde was observed by ¹H NMR spectroscopic analysis, 2,2-dimethyl-1,3-dioxan-5-one (8) (1.0 equiv.) in CH₂Cl₂ or THF or MeCN (1.25*DMF vol.) was then added and the resulting mixture was allowed to warm to room temperature. After a further 36-72 hours, or when complete consumption of 8 was observed by ¹H NMR spectroscopic analysis of small reaction aliquots, the mixture was diluted with CH₂Cl₂ and the organic layer was washed once with saturated sodium bicarbonate solution and once with water. The organic layer was then dried over MgSO₄, concentrated under reduced pressure and the crude product was purified by flash chromatography as indicated.

General Procedure B (Syn-Reduction)

To a stirred solution of syn- and anti-fluorohydrins (1.0 equiv) in MeCN (0.10 M) at −15° C. was added tetramethylammoniumtriacetoxyborohydride (5.0 equiv) and acetic acid (10 equiv). The resulting mixture was stirred 16 hours or until complete consumption of starting material (as determined by TLC analysis). The reaction mixture was then diluted with a saturated solution of Rochelle salt and washed three times with CH₂Cl₂. The organic layer was separated, dried over MgSO₄, concentrated under reduced pressure, and the crude product was purified by flash chromatography.

General Procedure C (Base Promoted Cyclization)

To a stirred solution of syn-diols, syn- and anti-fluorohydrins (1.0 equiv.) in MeCN (0.10 M) was added 2 M NaOH (2.5-10 equiv.) and the reaction mixture was stirred for 5 hours or until no starting material remained (as determined by TLC analysis). The reaction mixture was diluted with CH₂C₂ and washed with saturated ammonium chloride solution. The organic layer was separated, dried over MgSO₄, filtered, and concentrated under reduced pressure. The crude product was purified by flash chromatography.

General Procedure D (Lewis Acid Promoted Cyclization)

To a stirred solution of syn-diols, syn- and anti-fluorohydrin (1.0 equiv.) in MeCN (0.10 M) was added Sc(OTf)₃ or InCl₃ (0.10-2.5 equiv.) and the reaction mixture was stirred for 6 hours or until complete consumption of starting material (as determined by TLC analysis).

The reaction mixture was diluted with CH₂Cl₂ and was washed with saturated sodium bicarbonate solution. The organic layer was separated, dried over MgSO₄, filtered, and concentrated under reduced pressure. The crude product was purified by flash chromatography.

General Procedure E (Grignard Additions)

A stirred solution of fluorohydrin aldol adduct (1 equiv.) in CH₂Cl₂ (0.025 M) was cooled to −78° C. Organomagnesium reagent (2.2-5 equiv.) was added dropwise and the resulting reaction mixture was stirred for 5 hrs. The reaction mixture was quenched at −78° C. with an ammonium chloride:methanol solution (1:1—saturated ammonium chloride solution:methanol) and warmed to room temperature. The resulting mixture was diluted with CH₂Cl₂ and washed twice with water. The organic layer was dried over MgSO₄, filtered, and concentrated under reduced pressure to give crude product. The crude product was either purified by flash chromatography or used directly for cyclization.

Preparation and Characterization of Compounds

Preparation of S1, Aldehyde SM1, Aldol Adduct A1, Diol Adducts 18a/18b, and Nucleoside Analogues 17, 19, and 34

A solution of pyrazole (1.00 g, 14.7 mmol, 1.0 equiv.), bromoacetaldehyde diethyl acetal (2.67 mL, 17.6 mmol, 1.2 equiv.) and K₂CO₃ (4.06 g, 29.4 mmol, 2.0 equiv.) was stirred in DMF (74 mL) for 36 hours at 90° C. The reaction mixture was then filtered and washed with 40 mL of CH₂Cl₂ and concentrated under reduced pressure. Purification of crude S1 by flash chromatography (pentane:ethyl acetate—7:3) afforded SI (2.43 g, 90% yield) as a colorless oil. A solution of S1 (0.100 g, 0.543, 1.0 equiv.) was heated to 90° C. in 0.5 M HCl (0.54 mL) for 5 hrs. Upon complete conversion to SM1, the reaction mixture was concentrated under reduced pressure and the resulting product SM1 was used in the next reaction without purification.

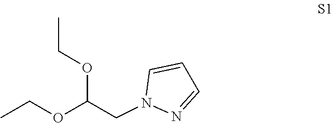

Data for S1: IR (neat): ν=2977, 2904, 1516, 1396, 1129, 1063, 751, 621 cm⁻¹; ¹H NMR (400 MHz, CDCl₃): δ 7.51 (d, J=1.8 Hz, 1H), 7.46 (d, J=2.3 Hz, 1H), 6.24 (dd, J=2.3, 1.8 Hz, 1H), 4.77 (t, J=5.5 Hz, 2H), 4.22 (d, J=5.5 Hz, 2H), 3.70 (m, 2H), 3.41 (m, 2H), 1.16 (t, J=7.1 Hz, 6H); ¹³C NMR (125 MHz, CDCl₃): δ 139.7, 130.6, 105.6, 101.7, 63.8, 55.2, 15.3 HRMS (EI⁺) calcd for C₉H₁₇N₂O₂ [M+H]⁺ 185.1285; found 185.1284

α-Fluorination/Aldol

Following General Procedure A, a solution of SM1 (0.543 mmol), NFSI (0.170 g, 0.543 mmol), L-proline (0.063 g, 0.543 mmol) and NaHCO₃ (0.045 g, 0.543 mmol) was stirred for 12 hours at 4° C. in DMF (0.72 mL). 8 (0.043 mL, 0.362 mmol) in MeCN (0.90 mL) was then added and the reaction mixture was stirred for 60 hrs at room temperature. Purification of the crude fluorohydrin A1 by flash chromatography (pentane:Et₂O—25:75) afforded a mixture of syn- and anti-fluorohydrins A1 (0.060 g, 64% yield, dr 1.4:1) as a light yellow oil.

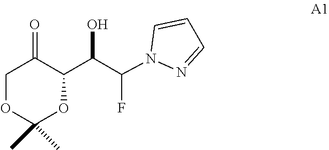

Data for syn- and anti-fluorohydrins A1: IR (neat): ν=2989, 1749, 1446, 1376, 1091, 1042, 764 cm⁻¹; ¹H NMR (600 MHz, CDCl₃): δ 7.88, 7.78, 7.63, 6.45, 6.44, 6.39, 6.37, 4.89, 4.50, 4.36, 4.34, 4.31, 4.26, 4.07, 4.04, 1.50, 1.45, 1.45, 1.34; ¹³C NMR (150 MHz, CDCl₃): δ 209.0, 207.4, 141.7, 141.4, 131.5, 131.1, 107.7, 107.5, 101.8, 101.4, 95.0, 94.6, 74.3, 72.4, 71.0, 70.2, 67.0, 66.9, 24.0, 23.7, 23.7, 23.4; ¹⁹F NMR (470 MHz, CDCl₃): δ −144.9, −154.1 HRMS (EI⁺) calcd for C₁₁H₁₆FN₂O₄ [M+H]⁺ 259.1089; found 259.1093

Syn-Reduction of Syn- and Anti-Fluorohydrins A1

Following General Procedure B, Me₄NHB(OAc)₃ (0.968 g, 3.68 mmol) and AcOH (0.442 mL, 7.36 mmol) were added to a stirred solution of A1 (0.190 g, 0.736 mmol) at −15° C. in MeCN (7.36 mL) and the reaction mixture was stirred for 18 hrs. Purification of the crude diols 18a and 18b by flash chromatography (pentane:ethyl acetate—1:1) afforded a mixture of 18a and 18b (0.151 g, 79% yield, d.r. (syn/anti)=1:1.2) as a colourless oil.

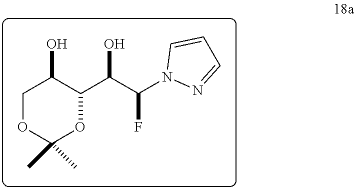

Data for syn-diol, syn-fluorohydrin 18a: [α]_{D}²⁰=+83.2 (c 0.37 in MeCN); IR (neat): ν=3001, 1442, 1375, 1039, 918, 749 cm⁻¹; ¹H NMR (600 MHz, CDCl₃): δ 7.68 (d, J=2.4 Hz, 1H), 7.64 (d, J=1.5 Hz, 1H), 6.38 (dd, J=2.4, 1.5 Hz, 1H), 6.18 (d, J=51.2 Hz, 1H), 4.27 (dd, J=22.4, 8.8 Hz, 1H), 3.95 (dd, J=11.1, 5.6 Hz, 1H), 3.93 (dd, J=9.5, 8.0 Hz, 1H), 3.80 (m, 1H), 3.70 (dd, J=11.2, 11.0 Hz, 1H), 1.52 (s, 3H), 1.39 (s, 3H); ¹³C NMR (150 MHz, CDCl₃): δ 141.5, 132.0, 107.2, 99.0, 91.9 (d, J=211.0 Hz), 72.3 (d, J=21.8 Hz), 70.6, 67.1, 63.8, 28.7, 19.4; ¹⁹F NMR (470 MHz, CD₃CN): δ −150.3 HRMS (EI⁺) calcd for C₁₁H₁₈FN₂O₄ [M+H]⁺ 261.1245; found 261.1255

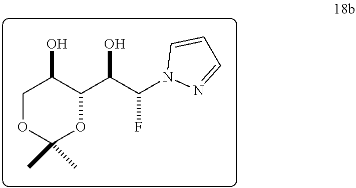

Data for syn-diol, anti-fluorohydrin 18b: [α]_{D}²⁰=−10.8 (c 0.91 in MeCN); IR (neat): ν=3646, 3001, 1443, 1375, 1039, 918 cm⁻¹; ¹H NMR (600 MHz, CDCl₃): δ 7.70 (d, J=0.9 Hz, 1H), 7.65 (d, J=2.5 Hz, 1H), 6.40 (dd, J=2.5, 0.9 Hz, 1H), 6.29 (dd, J=48.4, 2.9 Hz, 1H), 4.41 (ddd, J=8.0, 4.0, 2.9 Hz, 1H), 3.87 (m, 2H), 3.52 (dd, J=11.3, 2.7 Hz, 1H), 3.17 (dd, J=8.8, 8.8 Hz, 1H), 1.34 (s, 3H), 1.16 (s, 3H); ¹³C NMR (150 MHz, CDCl₃): δ 142.1, 132.0, 106.9, 98.9, 93.1 (d, J=207.9 Hz), 76.2 (d, J=24.7 Hz), 72.2 (d, J=5.3 Hz), 67.3 (d, J=4.6 Hz), 63.8, 28.5, 19.3; ¹⁹F NMR (470 MHz, CD₃CN): δ −145.9

HRMS (EI⁺) calcd for C₁₁H₁₈FN₂O₄[M+H]⁺ 261.1245 found 261.1262

Cyclization of Diols 18a and 18b

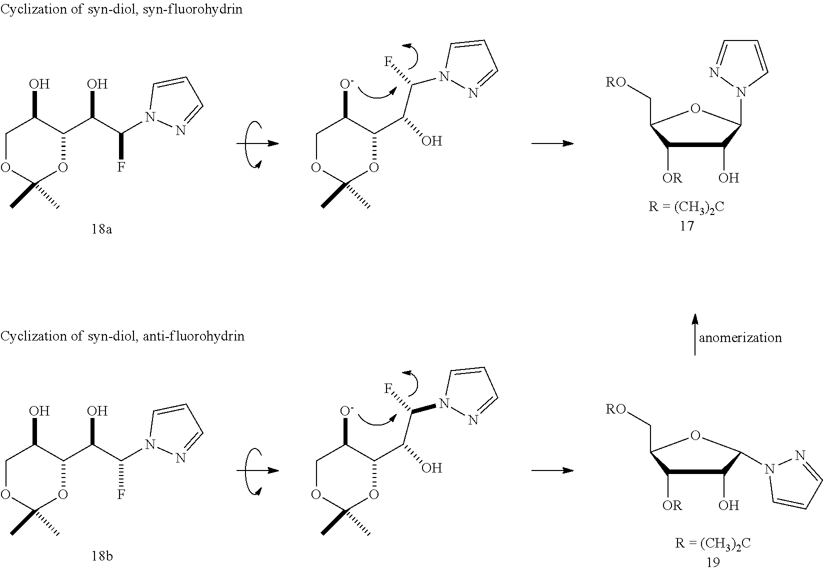

Following General Procedure C, diols 18a and 18b were cyclized separately to the same product (17). The α-anomer resulting from an S_{N}2 cyclization from 18b epimerizes following cyclization to the thermodynamically more stable β-anomer 17 under the reaction conditions. Moreover, taking a 2:1 mixture of products (19:17) and following General Procedure C affords only the β-anomer 17. Note also the e.r. of 17 (95:5) represents the average e.r. of 18a (93:7) and 18b (98:2).

Following General Procedure C, a mixture of 18a and 18b (0.025 g, 0.096 mmol, d.r. (syn/anti)=1:1) and 2 M NaOH (0.48 mL, 0.962 mmol) was stirred in MeCN (0.96 mL) at 50° C. for 5 hrs. Purification of the crude 34 by flash chromatography (pentane:ethyl acetate −65:35) afforded nucleoside analogue 34 (0.018 g, 76% yield) as a white solid. On occasion, product mixtures of up to 5:1 (β:α) were observed.

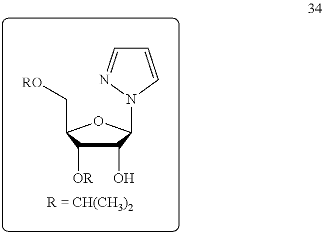

Data for nucleoside analogue 34: [α]_{D}²⁰=−58.9 (c 2.0 in MeCN); IR (neat): ν=3339, 2926, 1647, 1450, 1397, 1092, 1045, 759 cm⁻¹; ¹H NMR (400 MHz, CD₃CN): δ 7.70 (d, J=2.4 Hz, 1H), 7.56 (d, J=1.6 Hz, 1H), 6.30 (dd, J=2.4, 1.6 Hz, 1H), 5.70 (s, 1H), 4.47 (d, J=4.6 Hz, 1H), 4.12 (dd, J=9.6, 4.6 Hz, 1H), 4.11 (dd, J=9.6, 4.6 Hz, 1H), 3.91 (dd, J=10.3, 9.6 Hz, 1H), 3.83 (dd, J=9.6, 4.6 Hz, 1H), 3.72 (br s, 1H), 1.54 (s, 3H), 1.43 (s, 3H); ¹³C NMR (100 MHz, CD₃CN): δ 141.7, 130.1, 106.7, 101.7, 96.1, 74.7, 74.4, 71.8, 65.9, 29.3, 20.1 HRMS (EI⁺) calcd for C₁₁H₁₇N₂O₄ [M+H]⁺ 241.1183; found 241.1197

Deprotection of Nucleoside Analogue 34

34 (0.021 g, 0.088 mmol) was dissolved in MeOD (1.0 mL) and two drops of 1 M HCl was added and the solution was left for 12 hrs at room temperature. Subsequently, the reaction mixture was concentrated under reduced pressure to afford 17 as a white solid (0.018 g, 100%).

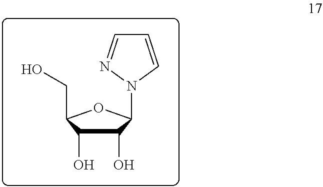

Data for nucleoside analogue 17: [α]_{D}²⁰=+70.4 (c 0.48 in MeOH); IR (neat): ν=3325, 2944, 2832, 1449, 1022, 631 cm⁻¹; ¹H NMR (600 MHz, CD₃CN): δ 7.74 (d, J=2.3 Hz, 1H), 7.58 (d, J=1.0 Hz, 1H), 6.30 (dd, J=2.3, 1.0 Hz, 1H), 5.70 (d, J=4.3 Hz, 1H), 4.51 (m, 1H), 4.33 (m, 1H), 4.08 (br s, 1H), 3.74 (dd, J=12.3, 2.8 Hz, 1H), 3.67 (d, J=5.7 Hz, 1H), 3.59 (dd, J=12.3, 2.5 Hz, 1H), 3.52 (d, J=4.3 Hz, 1H); ¹³C NMR (150 MHz, CD₃CN): δ 141.2, 131.1, 106.4, 94.7, 87.2, 76.6, 72.3, 63.4. HRMS (EI⁺) calcd for C₈H₁₃N₂O₄ [M+H]⁺ 201.0870; found 201.0870

Cyclization of Diol 18b

A solution of 18b (0.043 g, 0.165 mmol) and 2 M NaOH (0.21 mL, 0.443 mmol, 2.5 equiv.) was stirred for 3 hrs in MeCN (1.65 mL) at 50° C. Purification of the crude 19 by flash chromatography (pentane:ethyl acetate—65:35) afforded nucleoside analogue 19 (0.026 g, 76% yield) as a white solid.

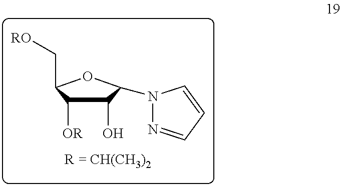

Data for nucleoside analogue 19: [α]_{D}²⁰=+72.2 (c 0.98 in MeCN); IR (neat): ν=3366, 2992, 1306, 1383, 1200, 1076, 754 cm⁻¹, 1H NMR (600 MHz, CD₃CN): δ 7.76 (d, J=2.3 Hz, 1H), 7.56 (d, J=1.2 Hz, 1H), 6.35 (d, J=2.3 Hz, 1H), 5.38 (d, J=0.9 Hz, 1H), 4.12 (dd, J=0.9, 2.1 Hz, 1H), 3.94 (d, J=2.1, 9.7 Hz, 1H), 3.81 (dd, J=5.0, 10.6 Hz, 1H), 3.59 (m, 2H), 3.37 (m), 1.45 (s, 3H), 1.33 (s, 3H); ¹³C NMR (150 MHz, CDCl₃): δ 142.1, 131.0, 108.2, 99.9, 71.8, 65.4, 65.2, 64.7, 59.0, 29.1, 19.9. HRMS (EI⁺) calcd for C₁₁H₁₇N₂O₄ [M+H]⁺ 241.1183; found 241.1176

Determination of Relative Stereochemistry for Diol 18a

Diol 18a was converted into the bis-p-nitro-benzoyl ester and recrystallized in ethanol. This allowed for the relative stereochemistry to be assigned using single X-ray crystallography.

Determination of Relative Stereochemistry for Nucleoside Analogue 17

Analysis of 2D NOESY of nucleoside analogue 17 supported the indicated stereochemistry.

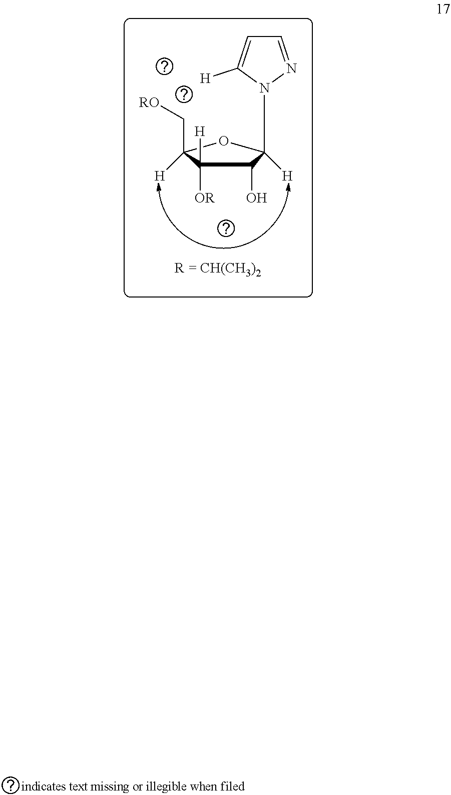

Determination of Relative Sterchemistry for Nucleoside Analogue 19

Analysis of 2D NOESY of nucleoside analogue 19 supported the indicated stereochemistry.

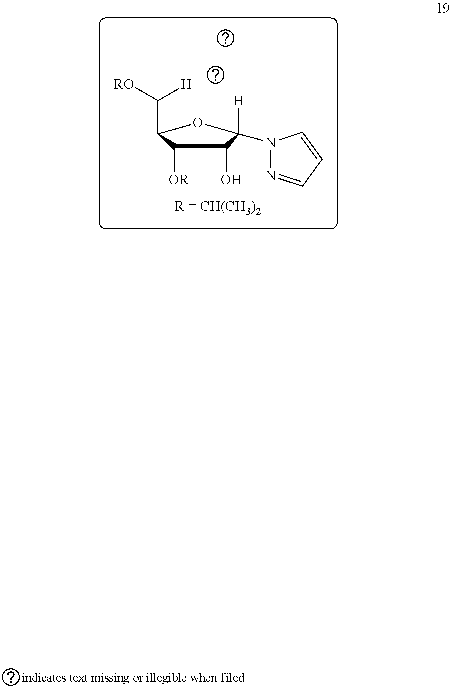

Determination of Enantiomeric Excess of Diol 18a

Following General Procedures A and B, using a 1:1 mixture of L-:D-proline, a racemic sample of diol 18a was prepared. The enantiomeric diols were separated by chiral HPLC using a Lux® 3 μm Amylose-1 column; flow rate 0.40 mL/min; eluent: hexanes-iPrOH 90:10; detection at 210 nm; retention time=6.66 min for (+)-18a; 8.10 min for (−)-18a. The enantiomeric ratio of the optically enriched (+)-18a diol was determined using the same method (93:7 e.r.).

Determination of Enantiomeric Excess of Diol 18b

Following General Procedures A and B, using a 1:1 mixture of L-:D-proline, a racemic sample of diol 18b was prepared. The enantiomeric diols were separated by chiral HPLC using a Lux® 3 μm Amylose-1 column; flow rate 0.40 mL/min; eluent: hexanes-iPrOH 90:10; detection at 210 nm; retention time=6.13 min for (−)-18b; 11.72 min for (+)-18b. The enantiomeric ratio of the optically enriched (−)-18b diol was determined using the same method (98:2 e.r.).

Determination of Enantiomeric Excess of Nucleoside Analogue 34

Following General Procedures A, B, and C, using a 1:1 mixture of L-:D-proline, a racemic sample of nucleoside 34 was prepared. The enantiomeric nucleosides were separated by chiral HPLC using a Lux® 3 μm-i-Cellulose-5 column; flow rate 0.10 mL/min; eluent: hexanes-iPrOH 90:10; detection at 254 nm; retention time=8.91 min for (−)-34; 13.32 min for (+)-34. The enantiomeric ratio of the optically enriched (−)-34 was determined using the same method (95:5 e.r.).

Preparation of Aldol Adduct A2, Diol Adducts D2, and Nucleoside Analogues 24, 35, and Ent-24

α-Fluorination/Aldol

The corresponding starting aldehyde/hydrate SM3 was prepared following literature procedures (45). Following General Procedure A, a solution of aldehyde (1.32 mmol), NFSI (0.416 g, 1.32 mmol), L-proline (0.152 g, 1.32 mmol) and NaHCO₃ (0.111 g, 1.32 mmol) was stirred for 12 hours at 4° C. in DMF (1.76 mL). 8 (0.105 mL, 0.880 mmol) in THF (2.64 mL) was then added and the reaction mixture was stirred for 96 hrs at 4° C. Purification of the crude fluorohydrin A2 by flash chromatography (pentane:ethyl acetate −1:1) afforded an inseparable mixture of syn- and anti-fluorohydrins A2 (0.159 g, 60% yield, d.r. 1.2:1) as an off-white solid.

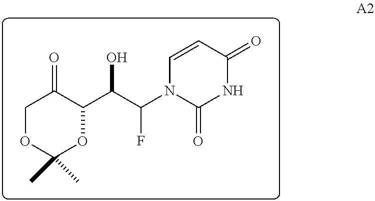

Data for syn- and anti-fluorohydrins A2: IR (neat): ν=3432, 2992, 2900, 1692, 1381, 1079 cm⁻¹; ¹H NMR (600 MHz, CDCl₃): δ 8.87, 8.79, 7.74, 7.68, 6.68, 6.67, 5.80, 5.77, 4.53, 4.40, 4.34, 4.33, 4.30, 4.13, 4.11, 4.06, 3.70, 3.48, 1.52, 1.46, 1.44, 1.44; ¹³C NMR (150 MHz, CDCl₃): δ 211.3, 208.7, 162.8, 162.6, 150.3, 149.8, 141.7, 141.1, 103.2, 102.6, 102.1, 101.9, 90.7, 90.3, 73.3, 71.4, 70.7, 70.5, 66.6, 66.5, 23.7, 23.6, 23.6, 23.3; ¹⁹F NMR (470 MHz, CDCl₃): δ −162.0, −178.6. HRMS (EI⁺) calcd for C₁₂H₁₆FN₂O₆[M+H]⁺ 303.0987; found 303.0982

Syn-Reduction of Syn- and Anti-Fluorohydrins A2

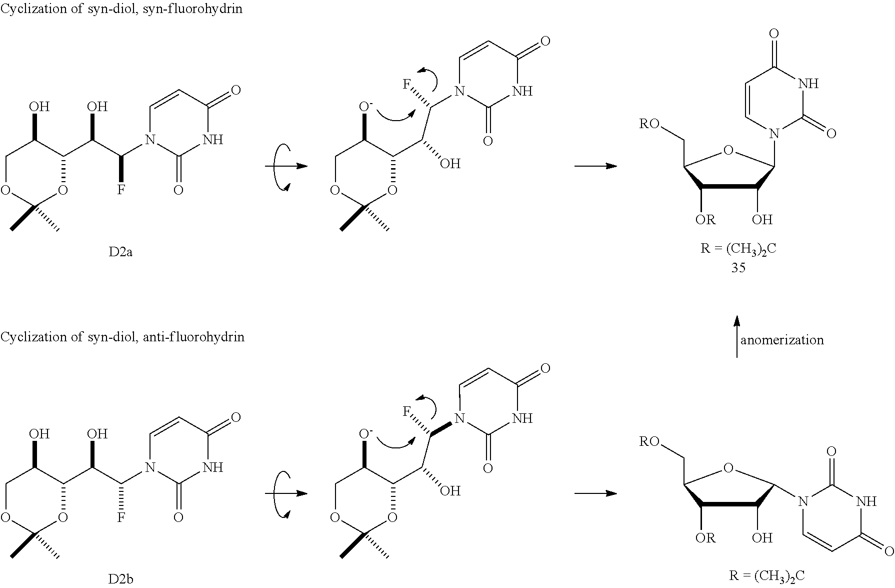

Following General Procedure 0, diols D2a and D2b were cyclized separately to the same product (35). The α-anomer resulting from an S_{N}2 cyclization from D2b epimerizes following cyclization to the thermodynamically more stable β-anomer 35.

Following General Procedure B, Me₄NHB(OAc)₃ (0.174 g, 0.660 mmol) and AcOH (0.076 mL, 1.32 mmol) were added to a stirred solution of A2 (0.040 g, 0.130 mmol) at −15° C. in MeCN (1.32 mL) and the reaction mixture was stirred for 24 hrs. Purification of the crude diols D2a and D21b by flash chromatography (pentane:ethyl acetate—1:3) afforded diols D2a and D21b (0.020 g, 50%, d.r. (syn/anti)=1.2:1) as white solids.

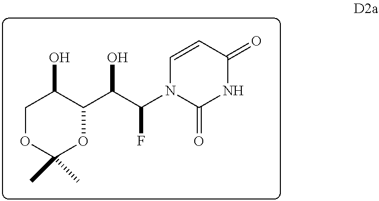

Data for syn-diol, syn-fluorohydrin D2a: ¹H NMR (600 MHz, MeOD): δ 7.76 (d, J=8.0, 1H), 6.46 (dd, J=44.4, 4.8 Hz, 1H), 5.73 (d, J=8.0 Hz, 1H), 4.03 (ddd, J=18.3, 7.0, 5.0 Hz, 1H), 3.82 (dd, J=11.4, 5.1 Hz, 1H), 3.71 (m, 2H), 3.60 (dd, J=11.4, 8.1 Hz, 1H), 1.42 (s, 3H), 1.28 (s, 3H); ¹³C NMR (150 MHz, MeOD): δ 165.8, 151.7, 143.1 (d, J=2.6 Hz), 102.9, 100.1, 94.3 (d, J=208.4 Hz), 74.6 (d, J=24.6 Hz), 73.7 (d, J=4.5 Hz), 67.3, 65.3, 28.3, 19.7. HRMS (EI⁺) calcd for C₁₂H₁₈FN₂O₆[M+H]⁺ 305.1143; found 305.1142

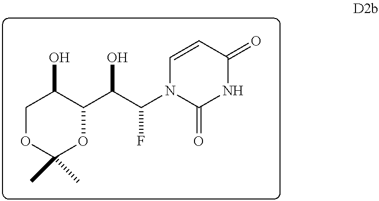

Data for syn-diol, anti-fluorohydrin D2b: ¹H NMR (600 MHz, MeOD): δ 7.90 (d, J=8.1 Hz, 1H), 6.71 (dd, J=44.2, 6.1 Hz, 1H), 5.74 (d, J=8.1 Hz, 1H), 4.32 (m, 1H), 3.81 (m, 3H), 3.60 (m, 1H), 1.43 (s, 3H), 1.32 (s, 3H); ¹³C NMR (150 MHz, MeOD): δ 165.8, 152.2, 143.0, 103.2 100.2, 92.6 (d, J=204.4), 75.9 (d, J=2.8 Hz), 71.5 (d, J=29.1 Hz), 65.7, 64.5 (d, J=2.2 Hz), 28.6, 19.4. HRMS (EI⁺) calcd for C₁₂H₁₈FN₂O₆[M+H]⁺ 305.1143; found 305.1123

Cyclization of Diols D2a and D2b

Following General Procedure C, a solution of D2 (0.022 g, 0.072 mmol, d.r. syn/anti=1.2:1) and 2 M NaOH (0.36 mL, 0.72 mmol) was stirred for 24 hours in MeCN (0.72 mL). Purification of the crude 35 by flash chromatography (CH₂Cl₂:MeOH—92.5:7.5) afforded nucleoside analogue 35 (0.019 g, 95% yield) as a white solid.

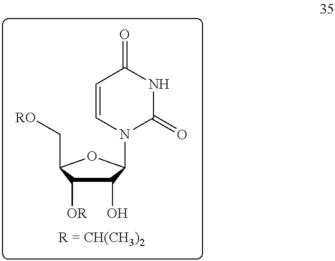

Data for nucleoside analogue 35: [α]_{D}²⁰=+48.1 (c 0.90 in MeOH); IR (neat): ν=2912, 1436, 1407, 1042, 952, 697 cm⁻¹; ¹H NMR (600 MHz, (CD₃)₂CO): δ 7.71 (d, J=8.0 Hz, 1H), 5.81 (s, 1H), 5.61 (d, J=8.0 Hz, 1H), 4.45 (d, J=4.6 Hz, 1H), 4.20 (dd, J=9.8, 4.7 Hz, 1H), 4.12 (dd, J=10.0, 10.0 Hz, 1H), 3.90 (dd, J=10.0, 4.8 Hz, 1H), 3.86 (ddd, J=10.0, 10.0, 4.7 Hz, 1H), 1.56 (s, 3H), 1.42 (s, 3H); ¹³C NMR (150 MHz, (CD₃)₂CO): δ 164.2, 151.8, 142.4, 103.4, 102.3, 94.5, 75.3, 74.6, 72.5, 66.1, 33.1, 22.8 HRMS (EI⁺) calcd for C₁₂H₁₇N₂O₆ [M+H]⁺ 285.1081; found 285.1085

Deprotection of Nucleoside Analogue 35

35 (0.019 g, 0.068 mmol) was dissolved in MeOD (0.68 mL) and two drops of 1 M HCl was added and the solution was left for 12 hrs at room temperature. Subsequently, the reaction mixture was concentrated under reduced pressure to afford nucleoside 24 as a white solid (0.017 g, 100%). The spectral data matched previous reports (46).

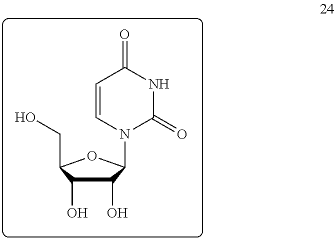

Data for nucleoside 24: [α]_{D}²⁰=−23 (c=0.1, MeOH); IR (neat): ν=3347, 2927, 2857, 1679, 1464, 1381, 1260, 1202, 1104, 1053, 806 cm⁻¹; ¹H NMR (600 MHz, MeOD): δ 8.03 (d, J=8.1 Hz, 1H), 5.91 (d, J=4.7 Hz, 1H), 5.70 (d, J=8.1 Hz, 1H), 4.18 (dd, J=4.9, 4.9 Hz, 1H), 4.15 (dd, J=4.9, 4.9 Hz, 1H), 4.00-4.01 (m, 1H), 3.84 (dd, J=12.2, 2.6 Hz, 1H), 3.74 (dd, J=12.2, 3.1 Hz, 1H); ¹³C NMR (150 MHz, MeOD): 166.2, 152.5, 142.7, 102.6, 90.6, 86.4, 75.7, 71.3, 62.3 HRMS (EI⁺) calcd for C₉H₁₃N₂O₆ [M+H]⁺ 245.0768; found 245.0770

Determination of Relative Stereochemistry for Diol D2a and D2b

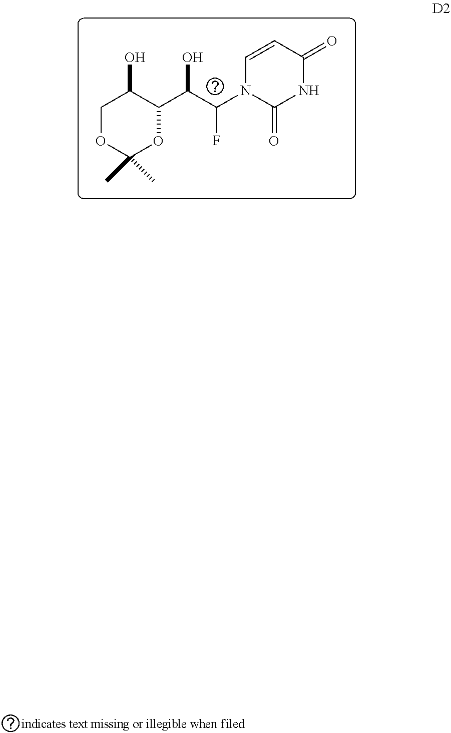

Based on J-based configurational analysis of compounds D5a/D5b, D8a/D8b and XRD analysis of compounds 18a, D7b, D9a a clear trend was established between the stereochemistry at the fluoromethine center and the chemical shift of the fluoromethine proton (*). In every case, the syn-fluorohydrin diol has a lower chemical shift than the diastereomeric anti-fluorohydrin diol. Here, D2a has a chemical shift of 6.46 ppm while D2b has a chemical shift of 6.71 ppm for the flouromethine proton. D2a was assigned as the syn-fluorohydrin diol and D2b the anti-fluorohydrin diol.

Determination of Relative Stereochemistry for Nucleoside 35

Analysis of 2D NOESY of nucleoside 35 revealed the indicated stereochemistry. Furthermore, the ¹H NMR and ¹³C NMR of nucleoside 24 matched reported data (38).

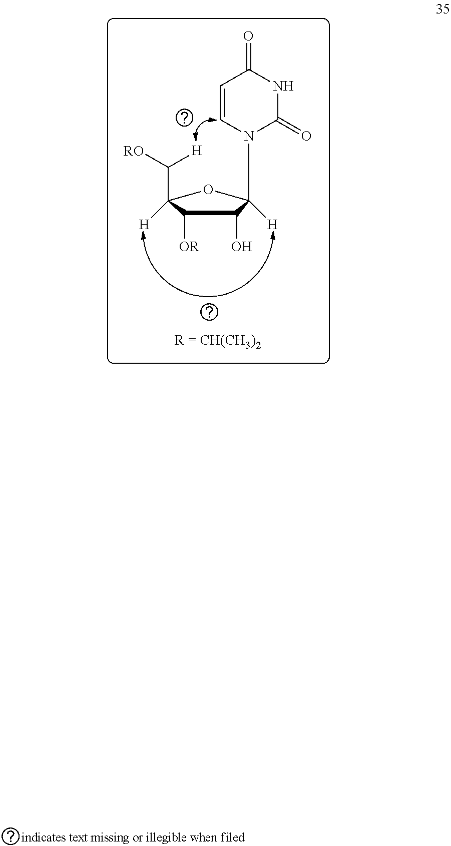

Determination of Enantiomeric Excess of Nucleoside Ent-35

Following General Procedures A, B, and C, using a 1:1 mixture of L-:D-proline, a racemic sample of nucleoside ent-35 was prepared. The enantiomeric nucleosides were separated by chiral HPLC using a Lux® 3 μm Amylose-1 column; flow rate 0.25 mL/min; eluent: hexanes-iPrOH 85:15; detection at 254 nm; retention time=19.99 min for (−)-35; 23.30 min for (+)-35. The enantiomeric ratio of the optically enriched ent-35 was determined using the same method (95:5 e.r.).

Preparation of Aldol Adducts A3, Diol Adducts D3, and Nucleoside Analogues NA3 and 25

α-Fluorination/Aldol

The corresponding starting aldehyde/hydrate SM3 was prepared following literature procedures (47). Following General Procedure A, a solution of SM3 (0.40 mmol), NFSI (0.126 g, 0.40 mmol), L-proline (0.046 g, 0.40 mmol) and NaHCO₃ (0.034 g, 0.40 mmol) was stirred for 14 hours at 4° C. in DMF (0.53 mL). Dioxanone 8 (0.032 mL, 0.27 mmol) in CH₂Cl₂ (0.67 mL) was then added and the reaction mixture was stirred for 96 hrs at 4° C. Purification of the crude fluorohydrin A3 by flash chromatography (pentane:ethyl acetate −3:7) afforded fluorohydrin A3 (0.072 g, 84% yield, d.r. 1.3:1) as an off-white solid. Mixture of 2 diastereomers and their corresponding tautomers (1:1.1:0.65:0.28). Varying the pH of the solution changes the ratio of these products. Following reduction, only 2 products (d.r. (syn/anti)=1.3:1) are present in the crude.

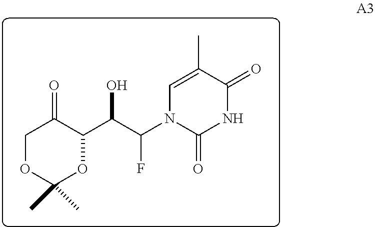

Data for syn- and anti-fluorohydrins A3: IR (neat): ν=2995, 1696, 1451, 1376, 1087, 1049 cm⁻¹; ¹H NMR (600 MHz, CDCl₃): δ 8.65, 8.60, 8.52, 7.57, 7.46, 7.41, 7.23, 6.67, 6.66, 6.64, 6.52, 4.59, 4.54, 4.52, 4.40, 4.39, 4.36, 4.35, 4.35, 4.33, 4.33, 4.32, 4.32, 4.12, 4.11, 4.07, 4.06, 3.67, 3.37, 1.97, 1.95, 1.95, 1.94, 1.52, 1.51, 1.51, 1.49, 1.47, 1.46, 1.45, 1.44; ¹³C NMR (150 MHz, CDCl₃): δ 211.4 208.5, 207.9, 206.4, 163.4, 163.2, 163.2, 163.1, 150.8, 150.5, 149.9, 149.9, 137.2, 136.2, 135.7, 134.6, 112.6, 112.0, 111.9, 111.0, 102.1, 102.1, 101.8, 101.7, 91.9, 90.8, 90.7, 90.1, 73.7, 73.0, 71.5, 70.8, 70.6, 70.5, 68.2, 68.0, 67.1, 66.8, 66.6, 66.5, 24.0, 23.9, 23.7, 23.7, 23.7, 23.6, 23.6, 23.4, 12.7, 12.7, 12.7, 12.7; ¹⁹F NMR (470 MHz, CDCl₃): δ −159.9, −161.6, −169.6, −177.8 HRMS (EI⁺) calcd for C₁₃H₁₈FN₂O₆ [M+H]⁺ 317.1143; found 317.1142

Syn-Reduction of Syn-Fluorohydrin and Anti-Fluorohydrins A3

Following General Procedure B, Me₄NHB(OAc)₃ (0.416 g, 1.58 mmol) and AcOH (0.181 mL, 3.16 mmol) were added to a stirred solution of A3 (0.100 g, 0.316 mmol) at −15° C. in MeCN (2.10 mL) and the reaction mixture was stirred for 18 hrs. Purification of the crude diol D3a by flash chromatography (pentane:ethyl acetate—3:7) afforded diols D3a and D3b (0.063 g, 63% yield, d.r. (syn:anti)=1.3:1) as a white solid.

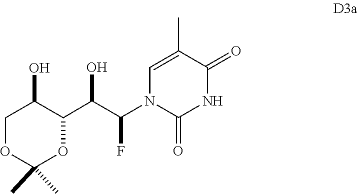

Data for syn-diol, syn-fluorohydrin D3a: [α]_{D}²⁰=−11.8 (c 1.0 in MeOH); IR (neat): ν=3363, 2924, 2858, 1674, 1380, 1209, 1075 cm⁻¹; ¹H NMR (600 MHz, CD₃CN): δ 7.42 (d, J=0.90 Hz, 1H), 6.36 (dd, J=44.9, 5.1 Hz, 1H), 4.04 (ddd, J=18.1, 6.6, 5.1 Hz, 1H), 3.79 (dd, J=11.3, 4.5 Hz, 1H), 3.67 (m, 2H), 3.55 (m, 1H), 1.83 (d, J=0.90 Hz, 3H), 1.39 (s, 3H), 1.24 (s, 3H); ¹³C NMR (150 MHz, CD₃CN): δ 164.7, 151.5, 137.9, 111.7, 99.9, 94.0 (d, J=205.9 Hz), 74.8 (d, J=25.1 Hz), 73.0 (d, J=4.3 Hz), 67.1, 65.0, 28.8, 19.9, 12.7; ¹⁹F NMR (470 MHz, CD₃CN): δ −169.1

¹H NMR in MeOD for syn-diol, syn-fluorohydrin D3a for relative stereochemical assignment: ¹H NMR (600 MHz, MeOD): δ 7.58 (s, 1H), 6.43 (dd, J=4.1 Hz, 1H), 4.06 (m, 1H), 3.81 (m 1H), 3.71 (m, 2H), 3.59 (m, 1H), 1.89 (s, 3H), 1.41 (s, 3H), 1.26 (s, 3H). HRMS (EI⁺) calcd for C₁₃H₂₀FN₂O₆[M+H]⁺ 319.1300; found 319.1329

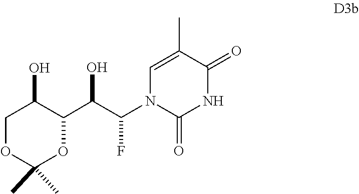

Data for syn-diol, anti-fluorohydrin D3b: [α]_{D}²⁰=+26.2 (c 0.45 in CH₃CN); IR (neat): ν=3360, 2922, 2855, 1670, 1380, 1207, 1078 cm⁻¹; ¹H NMR (600 MHz, MeOD): 7.72 (d, J=1.1 Hz, 1H), 6.71 (dd, J=44.3, 6.8 Hz, 1H), 4.32 (m, 1H), 3.82 (m, 3H), 3.60 (m, 1H), 1.90 (d, J=1.1 Hz, 3H), 1.44 (s, 3H), 1.32 (s, 3H); ¹³C NMR (150 MHz, MeOD): δ 166.1, 152.5, 138.3, 112.0, 100.2, 92.6 (d, J=204.7 Hz), 75.9, 71.3 (d, J=29.9 Hz), 65.7, 64.4 (d, J=2.1 Hz), 28.6, 19.5, 12.4. ¹⁹F NMR (470 MHz, CD₃CN): δ −160.3. HRMS (EI⁺) calcd for C₁₃H₂₀FN₂O₆[M+H]⁺ 319.1300; found 319.1320

Cyclization of Diols D3a and D3b

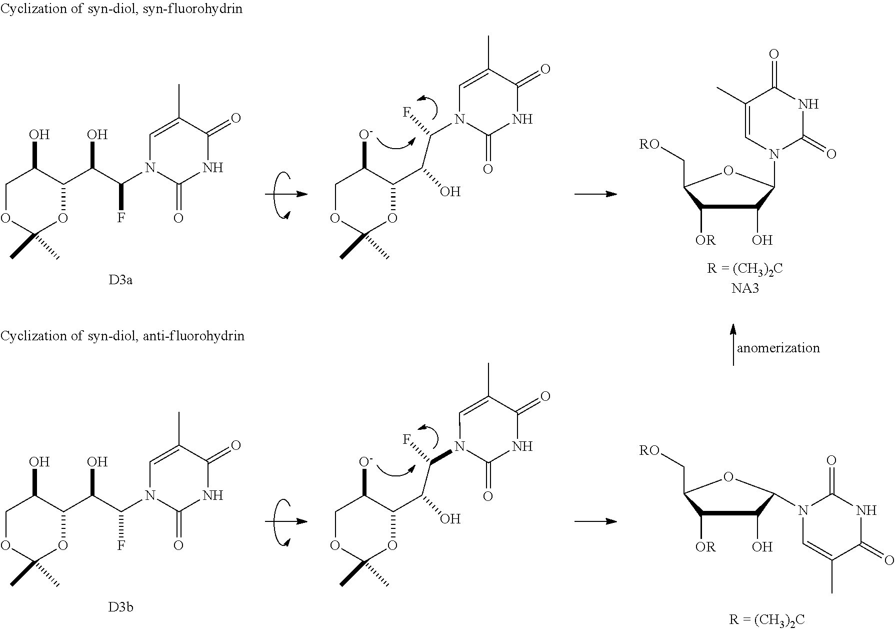

Following General Procedure C, diols D3a and D3b were cyclized separately to the same product, NA3. The α-anomer resulting from an S_{N}2 cyclization from D3b epimerizes following cyclization to the thermodynamically more stable β-anomer NA3.

Following General Procedure C, a solution of D3a and D3b (0.100 g, 0.314 mmol, d.r. syn/anti=1.5:1) and 2 M NaOH (0.236 mL, 0.472 mmol) was stirred for 10 hours in MeCN (3.14 mL). Purification of the crude nucleoside NA3 by flash chromatography (ethyl acetate) afforded nucleoside NA3 (0.089 g, 95% yield) as a white solid.

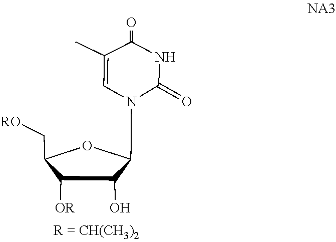

Data for nucleoside NA3: [α]_{D}²⁰=+39.4 (c 1.1 in MeCN); IR (neat): ν=3405, 2993, 1687, 1267, 1138, 845, 734 cm⁻¹; ¹H NMR (600 MHz, CD₃CN): δ 9.04 (br s, 1H), 7.19 (d, J=1.1 Hz, 1H), 5.67 (s, 1H), 4.22 (dd, J=4.8, 3.1 Hz, 1H), 4.15 (dd, J=9.1, 3.5 Hz, 1H), 4.02 (dd, J=10.1, 9.8 Hz, 1H), 3.70 (m, 2H), 3.55 (m, 1H), 1.85 (d, J=1.1 Hz, 3H), 1.53 (s, 3H), 1.41 (s, 3H); ¹³C NMR (150 MHz, CD₃CN): δ 164.9, 151.6, 137.5, 111.8, 102.3, 93.8, 74.7, 74.1, 72.1, 65.6, 29.6, 20.5, 12.7 HRMS (EI⁺) calcd for C₁₃H₁₉N₂O₆[M+H]⁺ 299.1238; found: 299.1277.

Deprotection of Nucleoside Analogue NA3

NA3 (0.010 g, 0.034 mmol) was dissolved in MeOD (0.34 mL) and two drops of 1 M HCl was added and the solution was left for 12 hrs at room temperature. Subsequently, the reaction mixture was concentrated under reduced pressure to afford 25 as a white solid (8.7 mg, 100%). The spectral data matched previous reports (48).

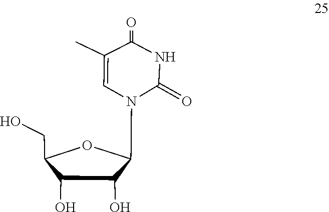

Data for nucleoside analogue 25: [α]_{D}²⁰=−33.0 (c=0.1 in MeOH); IR (neat): ν=3346, 2928, 2867, 1688, 1466, 1378, 1262, 1200, 1104, 1050, 803 cm⁻¹; ¹H NMR (600 MHz, MeOD): δ 7.86 (d, J=1.1 Hz, 1H), 5.91 (d, J=4.6 Hz, 1H), 4.15-4.18 (m, 2H), 3.98-4.00 (m, 1H), 3.86 (dd, J=12.2, 2.7 Hz, 1H), 3.75 (dd, J=12.2, 3.0 Hz, 1H), 1.88 (d, J=0.9 Hz, 3H); ¹³C NMR (150 MHz, MeOD): δ 166.4, 152.7, 138.4, 111.5, 90.3, 86.3, 75.5, 71.3, 62.3, 12.4. HRMS (EI⁺) calcd for C₁₀H₁₅N₂O₆ [M+H]⁺ 259.0925; found: 259.0923.

Determination of Relative Stereochemistry for Diol D3a and D3b

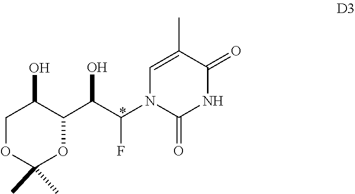

Based on J-based configurational analysis of compounds D5a/D5b, D8a/D8b and XRD analysis of compounds 18a, D7b, D9a a clear trend was established between the stereochemistry at the fluoromethine center and the chemical shift of the fluoromethine proton (*). In every case, the syn-fluorohydrin diol has a lower chemical shift than the diastereomeric anti-fluorohydrin diol. Here, D3a has a chemical shift of 6.43 ppm while D3b has a chemical shift of 6.69 ppm for the fluoromethine proton. D3a was assigned as the syn-fluorohydrin diol and D3b the anti-fluorohydrin diol.

Determination of Absolute Stereochemistry

Comparison of [α]_{D}²⁰ values of nucleoside 25 with literature values confirmed absolute stereochemistry (49).

Determination of Enantiomeric Excess of Nucleoside NA3

Following General Procedures A, B, and C, using a 1:1 mixture of L-:D-proline, a racemic sample of nucleoside NA3 was prepared. The enantiomeric nucleosides were separated by chiral HPLC using a Lux® 3 μm Amylose-1 column; flow rate 0.25 mL/min; eluent: hexanes-iPrOH 85:15; detection at 254 nm; retention time=5.18 min for (+)-NA3; 12.61 min for (−)-NA3. The enantiomeric ratio of the optically enriched (+)-NA3 was determined using the same method (91:9 e.r.).

Preparation of Aldol Adduct A4, Diol Adducts D4a/D4b, and Nucleoside Analogue 27

α-Fluorination/Aldol and Syn-Reduction of Syn- and Anti-Fluorohydrins

Following General Procedure A, a solution of 2-(4,6-dichloropyrimidin-5-yl)acetaldehyde (0.250 g, 1.31 mmol, 1 equiv.), NFSI (0.413 g, 1.31 mmol, 1 equiv.), L-proline (0.151 g, 1.31 mmol, 1 equiv.) and NaHCO₃ (0.110 g, 1.31 mmol, 1 equiv.) was stirred for 1 hr at 4° C. in DMF (1.19 mL). Dioxanone 8 (0.521 mL, 4.36 mmol, 3.33 equiv.) was added and the reaction mixture was stirred for 24 hrs at 4° C. Purification of the crude fluorohydrin A4 by flash chromatography (pentane:ethyl acetate—3:7) afforded fluorohydrin A4 (0.301 g, 68% yield) as an orange oil. Following General Procedure B, Me₄NHB(OAc)₃ (2.16 g, 8.21 mmol) and AcOH (0.905 mL, 16.4 mmol) were added to a stirred solution of A4 (0.555 g, 1.64 mmol) at −15° C. in MeCN (16.4 mL) and the reaction mixture was stirred for 24 hrs. Purification of the crude diol D4a by flash chromatography (pentane:ethyl acetate—4:1) afforded diol D4a (0.295 g, 53% yield, d.r. (syn/anti)=3:1) as an off-white solid.

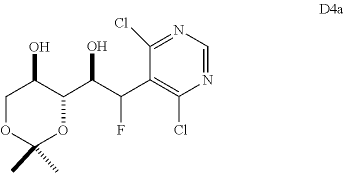

Data for syn-diol D4a: [α]_{D}²⁰=+26.6 (c 5.0 in MeCN); IR (neat): ν=3000, 1442, 1375, 1039, 918, cm⁻¹; ¹H NMR (600 MHz, CDCl₃): δ 8.73 (s, 1H), 6.05 (dd, J=46.0, 7.9 Hz, 1H), 4.64 (m, 1H), 3.89 (dd, J=11.5, 5.7 Hz, 1H), 3.80 (m, 1H), 3.73 (dd, J=9.1, 8.5 Hz, 1H), 3.61 (dd, J=11.5, 9.5 Hz, 1H), 1.29 (s, 3H), 0.94 (s, 3H); ¹³C NMR (150 MHz, CDCl₃): δ 161.5, 157.4, 127.8, 98.3, 91.1 (d, J=179.4 Hz), 75.5 (d, J=21.3 Hz), 71.7 (d, J=5.5 Hz), 66.6, 63.3, 28.2, 18.7; ¹⁹F NMR (470 MHz, CDCl₃): δ −193.0. HRMS (EI⁺) calcd for C₁₂H₁₆C₁₂FN₂O₄[M+H]⁺ 341.0466; found 341.0425

Cyclization of Diol D4a

Following General Procedure C, a solution of D4a (0.014 g, 0.044 mmol, 1 equiv.) and 2 M NaOH (0.11 mL, 0.22 mmol, 5 equiv.) was stirred for 15 minutes in MeCN (0.30 mL). Purification of the crude nucleoside 27 by flash chromatography (ethyl acetate:pentane—50:50) afforded nucleoside 27 (6.4 mg, 51% yield) as a white solid.

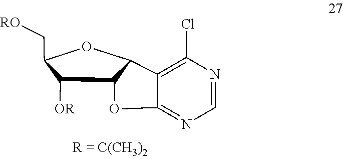

Data for nucleoside analogue 27: [α]_{D}²⁰=+51.2 (c 0.34 in CH₂Cl₂); IR (neat): ν=3363, 2927, 1602, 1598, 1571, 1408, 968 cm⁻¹; ¹H NMR (600 MHz, CD₃CN): δ 8.66 (s, 1H), 4.19 (dd, J=10.1, 4.9 Hz, 1H), 3.91 (dd, J=10.2, 10.1 Hz, 1H), 3.86 (dd, J=10.1, 4.7 Hz, 1H), 3.30 (ddd, J=10.2, 10.1, 4.8 Hz, 1H), 1.56 (s, 3H), 1.51 (s, 3H); ¹³C NMR (150 MHz, CD₃CN): δ 176.6, 160.8, 158.7, 114.6, 101.7, 82.2, 79.1, 75.6, 69.0, 64.7, 28.9, 19.5. HRMS (EI⁺) calcd for C₁₂H₁₄ClN₂O₄[M+H]⁺ 285.0637; found 285.0644

Determination of the Relative Stereochemistry for Nucleoside 27

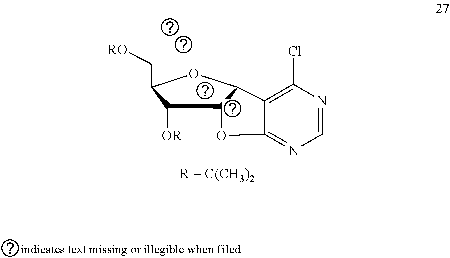

Analysis of 2D NOESY of nucleoside 27 revealed the indicated stereochemistry.

Determination of Enantiomeric Excess of Diol D4a

Following General Procedures A and B, using a 1:1 mixture of L-:D-proline, a racemic sample of diol D4a was prepared. The enantiomeric diols were separated by chiral HPLC using a Lux® 3 μm Amylose-1 column; flow rate 0.25 mL/min; eluent: hexanes-iPrOH 90:10; detection at 254 nm; retention time=11.81 min for (−)-D4a; 12.68 min for (+)-D4a. The enantiomeric ratio of the optically enriched (+)-D4a diol was determined using the same method (95:5 e.r.).

Preparation of S5, Hydrate SM5, Aldol Adduct A5, Diol Adducts D5a and D5b, and Nucleoside Analogue 28

A solution of 1,2,3-triazole (1.00 mL, 17.2 mmol, 1.0 equiv.), bromoacetaldehyde diethyl acetal (3.10 mL, 20.7 mmol, 1.2 equiv.) and K₂CO₃ (4.75 g, 34.4 mmol, 2.0 equiv.) was stirred for 24 hours at 90° C. in DMF (86 mL). The reaction mixture was then filtered and washed with 40 mL of CH₂Cl₂ and concentrated under reduced pressure. Purification of crude S5 by flash chromatography (pentane:ethyl acetate—7:3) afforded S5 (2.90 g, 91% yield) as a colorless oil. A solution of S5 (0.100 g, 0.54 mmol, 1.0 equiv.) was heated to 90° C. in 0.5M HCl (0.54 mL) for 5 hours. Upon complete conversion to SM5, the reaction mixture was concentrated under reduced pressure and the resulting product SM5 was used in the reaction without purification.

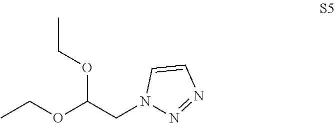

Data for S5: ¹H NMR (400 MHz, CDCl₃): δ 7.68 (d, J=0.90 Hz, 1H), 7.66 (d, J=0.90 Hz, 1H), 4.76 (t, J=5.3 Hz, 1H), 4.48 (d, J=5.3 Hz, 2H), 3.73 (m, 2H), 3.47 (m, 2H), 1.17 (m, 6H); ¹³C NMR (125 MHz, CDCl₃): δ 133.8, 124.9, 101.1, 64.0, 52.9, 15.3. HRMS (EI⁺) calcd for C₈H₁₆N₃O₂ [M+H]⁺ 186.1237; found 186.1233

α-Fluorination/Aldol

Following General Procedure A, a solution of S5 (0.54 mmol), Selectfluor (0.192 g, 0.54 mmol), L-proline (0.063 g, 0.54 mmol) and NaHCO₃ (0.045 g, 0.54 mmol) was stirred for 12 hours at 4° C. in DMF (0.72 mL). Dioxanone 8 (0.043 mL, 0.36 mmol) in MeCN (0.43 mL) was then added and the reaction mixture was stirred for 72 hrs at room temperature. Purification of the crude fluorohydrin A5 by flash chromatography (Et₂O) afforded fluorohydrin A5 (0.061 g, 65% yield, d.r. 1:1) as a light yellow oil.

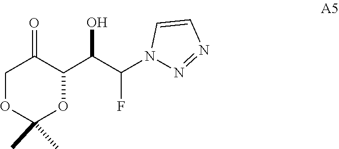

Data for syn- and anti-fluorohydrins A5: IR (neat): ν=3138, 2990, 1749, 1455, 1379, 1224, 1070, 799 cm⁻¹; ¹H NMR (600 MHz, CDCl₃): δ 8.24 (1H), 8.12 (1H), 7.79 (1H), 7.77 (1H), 6.89 (1H), 6.86 (1H), 4.74 (1H), 4.49 (1H), 4.33 (2H), 4.26 (1H), 4.14 (1H), 4.06 (1H), 3.89 (1H), 1.55 (3H), 1.48 (3H), 1.44 (3H), 1.31 (3H); ¹³C NMR (150 MHz, CDCl₃): δ 210.8, 209.4, 134.5, 134.5, 124.4, 124.4, 102.1, 102.0, 94.5, 93.5, 72.1, 71.3, 70.8, 70.1, 66.5, 66.5, 23.8, 23.5, 23.4, 23.4; ¹⁹F NMR (470 MHz, CDCl₃): δ −154.6, −163.8. HRMS (EI⁺) calcd for C₁₀H₁₅FN₃O₄[M+H]⁺ 260.1041; found 260.1044

Syn-Reduction of Syn- and Anti-Fluorohydrins A

Following General Procedure B, Me₄NHB(OAc)₃ (0.391 g, 1.49 mmol) and AcOH (0.170 mL, 2.98 mmol) were added to a stirred solution of A5 (0.077 g, 0.30 mmol) at −15° C. in MeCN (3.00 mL) and the reaction mixture was stirred for 24 hrs. Purification of the crude diols D5a and D5b by flash chromatography (CH₂Cl₂:MeOH—96:4) afforded diols D5a and D5b (0.072 g, 94% yield, d.r. (syn/anti)=1.2:1) as white solids.

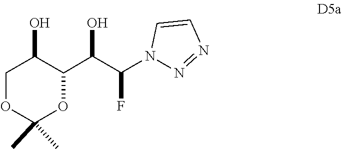

Data for syn-diol, syn-fluorohydrin D5a: [α]_{D}²⁰=+52.4 (c 0.51 in MeCN); IR (neat): ν=3432, 2997, 2253, 1444, 1375, 1071, 1039 cm⁻¹; ¹H NMR (600 MHz, CD₃CN): δ 8.17 (d, J=1.0 Hz, 1H), 7.78 (d, J=1.0 Hz, 1H), 6.69 (dd, J=48.1, 4.7 Hz, 1H), 4.36 (ddd, J=18.4, 5.0, 5.0 Hz, 1H), 3.79 (dd, J=11.4, 5.0 Hz, 1H), 3.63 (m, 2H), 3.54 (m, 2H), 1.39 (s, 3H), 1.31 (s, 3H); ¹³C NMR (150 MHz, CD₃CN): δ 135.2, 126.2, 100.0, 95.9 (d, J=206.7 Hz), 74.7 (d, J=22.7 Hz), 73.1 (d, J=4.4 Hz), 66.0, 65.2, 28.8, 19.9; ¹⁹F NMR (470 MHz, CDCl₃): δ −156.0 HRMS (EI⁺) calcd for C₁₀H₁₇FN₃O₄[M+H]1 262.1198; found 262.1209.

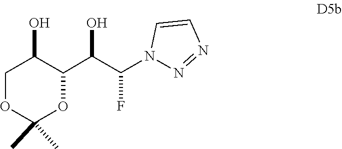

Data for syn-diol, anti-fluorohydrin D5b: [α]_{D}²⁰=+40.0 (c 0.37 in MeCN); IR (neat): ν=3000, 1442, 1375, 1039, 918, 740 cm⁻¹; ¹H NMR (600 MHz, CD₃CN): δ 8.22 (d, J=1.0 Hz, 1H), 7.79 (d, J=1.0 Hz, 1H), 6.78 (dd, J=46.4, 6.0 Hz, 1H), 4.53 (ddd, J=10.4, 6.0, 4.7 Hz, 1H), 4.09 (br s, 1H), 3.83 (m, 2H), 3.57 (m, 2H), 3.41 (br s, 1H), 1.35 (s, 3H), 1.34 (s, 3H); ¹³C NMR (150 MHz, CD₃CN): δ 135.3, 125.7, 100.0, 96.5 (d, J=204.3 Hz), 74.2 (d, J=2.3 Hz), 72.9 (d, J=27.2 Hz), 65.4, 65.3 (d, J=2.0 Hz), 28.9, 19.8; ¹⁹F NMR (470 MHz, CDCl₃): δ −151.2 HRMS (EI⁺) calcd for C₁₀H₁₇FN₃O₄[M+H]⁺ 262.1198; found 262.1206

Cyclization of Diol D5a

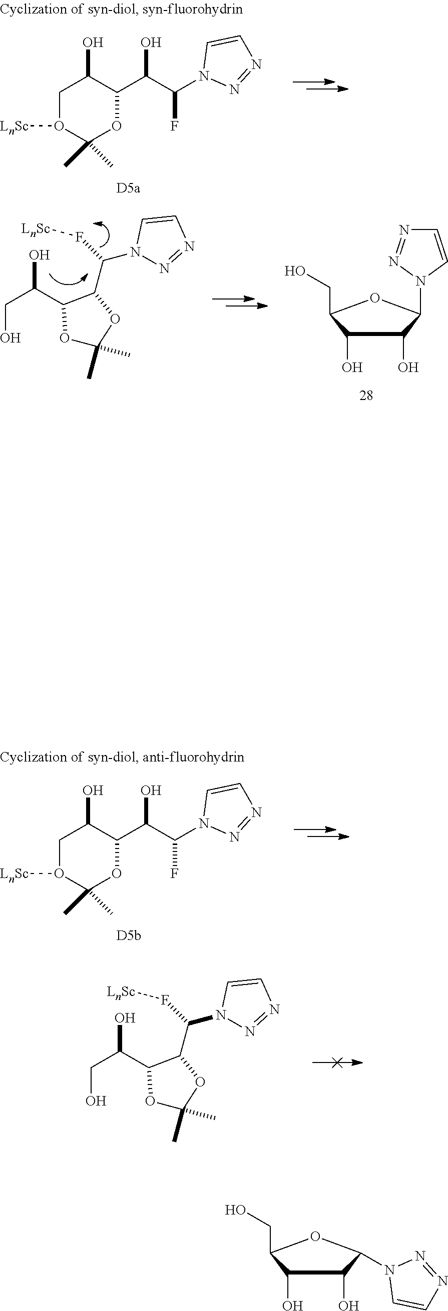

Following General Procedure D, diol D5a was cyclized separately to 28 while diol D5b did not cyclize. This suggests the product generated from the diol mixture comes only from the D5a diol via an S_{N}2 cyclization.

Following General Procedure D, a solution of D5a and D5b (0.025 g, 0.096 mmol, 1.0 equiv, d.r. (syn/anti)=1.2:1) and Sc(OTf)₃ (0.118 g, 0.239 mmol, 2.5 equiv.) was stirred in dry MeCN (1.00 mL). After 12 hours, pyridine (0.50 mL) and acetic anhydride (0.25 mL) were added and the reaction mixture was left to stir for 3 hrs. Purification of the crude 28 by flash chromatography (pentane:ethyl acetate—1:3) afforded nucleoside analogue 28 (0.015 g, 47% yield) as a clear colorless oil.

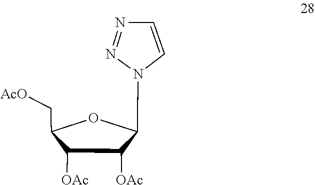

Data for nucleoside analogue 28: [α]_{D}²⁰=+1.3 (c 0.60 in CH₂Cl₂); IR (neat): ν=2926, 1747, 1373, 1227, 1064 cm⁻¹; ¹H NMR (600 MHz, CDCl₃): δ 7.76 (s, 1H), 7.26 (s, 1H), 6.19 (d, J=3.7 Hz. 1H), 5.85 (dd, J=5.0, 3.8 Hz, 1H), 5.63 (dd, J=5.3, 5.0 Hz, 1H), 4.49 (ddd, J=5.3, 4.3, 3.0 Hz, 1H), 4.41 (dd, J=12.4, 3.0 Hz, 1H), 4.22 (dd, J=12.4, 4.3 Hz, 1H), 2.13 (s, 3H), 2.13 (s, 3H), 2.06 (s, 3H); ¹³C NMR (150 MHz, CDCl₃): δ 170.5, 169.6, 169.5, 134.3, 122.9, 90.0, 81.0, 74.5, 70.8, 62.9, 20.8, 20.6, 20.6; HRMS (EI⁺) calcd for C₁₃H₁₈N₃O₇ [M+H]⁺ 328.3005; found 328.3000

Determination of Relative Stereochemistry for Diol D5a

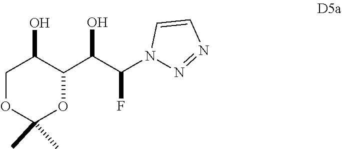

The relative stereochemistry of diol D5a was determined by J-based configurational analysis. See J-based configurational analysis section for details.

Determination of Relative Stereochemistry for Diol D5b

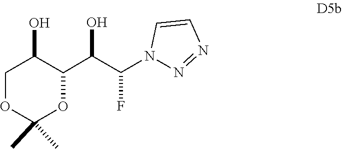

The relative stereochemistry of diol D5b was determined by J-based configurational analysis. See J-based configurational analysis section for details.

Determination of Relative Stereochemistry for Nucleoside 28

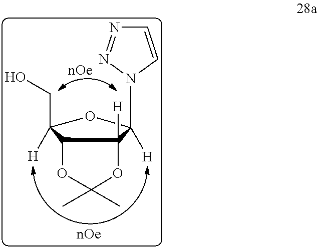

Analysis of 2D NOESY of nucleoside 28a supported the indicated stereochemistry.

Determination of Enantiomeric Excess of Diol D5a

Following General Procedures A and B, using a 1:1 mixture of L-:D-proline, a racemic sample of diol D5a was prepared. The enantiomeric diols were separated by chiral HPLC using a Lux® 3 μm i-Cellulose-5 column; flow rate 0.20 mL/min; eluent: hexanes-iPrOH 90:10; detection at 210 nm; retention time=4.69 min for (+)-D5a; 5.80 min for (−)-D5a. The enantiomeric ratio of the optically enriched (+)-D5a diol was determined using the same method (93:7 e.r.).

Determination of Enantiomeric Excess of Diol D5b

Following General Procedures A and B, using a 1:1 mixture of L-:D-proline, a racemic sample of diol D5b was prepared. The enantiomeric diols were separated by chiral HPLC using a Lux® 3 μm i-Cellulose-5 column; flow rate 0.20 mL/min; eluent: hexanes-iPrOH 90:10; detection at 210 nm; retention time=3.94 min for (−)-D5b; 4.95 min for (+)-D5b. The enantiomeric ratio of the optically enriched (+)-D5b diol was determined using the same method (96:4 e.r.).

Determination of Enantiomeric Excess of Diols Ent-D5a

Following General Procedures A and B, using a 1:1 mixture of L-:D-proline, a racemic sample of diol ent-D5a was prepared. The enantiomeric diols were separated by chiral HPLC using a Lux® 3 μm i-Cellulose-5 column; flow rate 0.20 mL/min; eluent: hexanes-iPrOH 90:10; detection at 210 nm; retention time=4.69 min for (+)-D5a; 5.80 min for (−)-D5a. The enantiomeric ratio of the optically enriched ent-D5a diol was determined using the same method (95:5 e.r.).

Determination of Enantiomeric Excess of Diols Ent-D5b

Following General Procedures A and B, using a 1:1 mixture of L-:D-proline, a racemic sample of diol ent-D5b was prepared. The enantiomeric diols were separated by chiral HPLC using a Lux® 3 μm i-Cellulose-5 column; flow rate 0.20 mL/min; eluent: hexanes-iPrOH 90:10; detection at 210 nm; retention time=3.94 min for (−)-D5b; 4.95 min for (+)-D5b. The enantiomeric ratio of the optically enriched ent-D5b diol was determined using the same method (95:5 e.r.).

Preparation of S6, Hydrate SM6, Aldol Adduct A6, Diol Adducts D6a and D6b, and Nucleoside Analogue 29

A solution of trifluoromethyluracil (1.00 g, 5.52 mmol, 1.0 equiv.), bromoacetaldehyde diethyl acetal (1.66 mL, 11.1 mmol, 2.0 equiv.) and K₂CO₃ (1.53 g, 11.1 mmol, 2.0 equiv.) was stirred for 24 hours at 90° C. in DMF (27.6 mL). The reaction mixture was then filtered and washed with 40 mL of CH₂Cl₂ and concentrated under reduced pressure. Purification of crude S6 by flash chromatography (pentane:ethyl acetate—7:3) afforded S6 (0.605 g, 37% yield) as a colorless oil. A solution of S7 (0.100 g, 0.340 mmol, 1.0 equiv.) was heated to 90° C. in 0.5 M HCl (0.34 mL) for 5 hours. Upon complete conversion to aldehyde/hydrate SM6, the reaction mixture was concentrated under reduced pressure and the resulting aldehyde/hydrate SM6 was used in the reaction without purification.

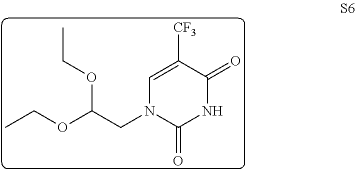 [SMILES: CCOC(Cn1cc(C(F)(F)F)c(=O)[nH]c1=O)OCC]

Data for S6: IR (neat): ν=3430, 2988, 2800, 1109, 1025 cm⁻¹; ¹H NMR (600 MHz, CDCl₃): δ 8.56 (br s, 1H), 7.82 (s, 1H), 4.61 (t, J=5.0 Hz), 3.88 (d, J=5.0 Hz), 3.78 (m, 2H), 3.54 (m, 2H), 1.21 (m, 6H); ¹³C NMR (150 MHz, CDCl₃): δ 158.6, 150.0, 147.0 (q, J=5.8 Hz), 121.9 (q, J=270.5 Hz), 104.7 (q, J=33.5 Hz), 100.0, 64.6, 51.0, 15.3. HRMS (EI⁺) calcd for C₁₁H₁₆F₃N₂O₄[M+H]⁺ 297.1057; found 297.1056

α-Fluorination/Aldol

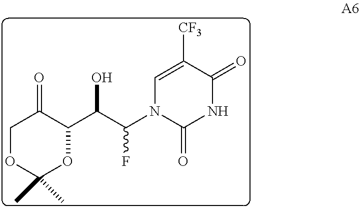

Following General Procedure A, a solution of SM6 (0.340 mmol), NFSI (0.107 g, 0.340 mmol), L-proline (0.039 g, 0.340 mmol) and NaHCO₃ (0.029 g, 0.340 mmol) was stirred for 12 hours at 4° C. in DMF (0.45 mL). Dioxanone 8 (0.027 mL, 0.227 mmol) in CH₂Cl₂ (0.57 mL) was then added and the reaction mixture was stirred for 96 hrs at 4° C. Purification of the crude fluorohydrin A6 by flash chromatography (pentane:ethyl acetate—65:35) afforded fluorohydrin A6 (0.050 g, 60% yield) as a light yellow oil.

Data for syn- and anti-fluorohydrins A6: IR (neat): ν=2991, 1699, 1450, 1087, 1049 cm⁻¹; ¹H NMR (600 MHz, CD₃CN): δ 9.53, 9.52, 8.15, 8.11, 6.58, 6.46, 4.62, 4.56, 4.55, 4.43, 4.31, 4.29, 3.98, 3.98, 1.43, 1.40, 1.40, 1.38; ¹³C NMR (150 MHz, CD₃CN): δ 208.4, 207.9, 159.6, 159.5, 150.6, 150.1, 144.0, 144.0, 123.6, 123.5, 106.6, 106.0, 102.4, 102.3, 95.3, 92.4, 76.3, 76.1, 69.9, 69.1, 67.9, 67.8, 24.5, 24.4, 24.2, 23.9; ¹⁹F NMR (470 MHz, CD₃CN): δ −64.1, −64.1, −161.4, −169.1. HRMS (EI⁺) calcd for C₁₃H₁₄F₄N₂NaO₆ [M+Na]⁺ 393.0680; found 393.0682

Syn-Reduction of Syn- and Anti-Fluorohydrins A6

Following General Procedure B, Me₄NHB(OAc)₃ (0.355 g, 1.35 mmol) and AcOH (0.155 mL, 2.79 mmol) were added to a stirred solution of A6 (0.100 g, 0.27 mmol, 1 equiv.) at −15° C. in MeCN (1.80 mL) and the reaction mixture was stirred for 24 hrs. Purification of the crude diols D6a and D6b by flash chromatography (pentane:ethyl acetate—4:1) afforded diols D6a (0.040 g, 40% yield) and D6b (0.019 g, 19% yield) as white solids.

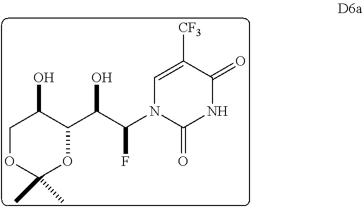

Data for syn-diol, syn-fluorohydrin D6a: [α]_{D}²⁰=+18.4 (c 0.50 in CH₂Cl₂); IR (neat): ν=3426, 2996, 1702, 1463, 1379, 1070 cm⁻¹; ¹H NMR (600 MHz, CD₃CN): δ 9.42 (br s, 1H), 8.10 (s, 1H), 6.33 (dd, J=45.1, 5.6 Hz, 1H), 4.28 (dd, J=14.8, 5.6 Hz, 1H), 3.79 (dd, J=11.1 5.5 Hz, 1H), 3.70 (m, 2H), 3.60 (dd, J=9.5, 2.7 Hz, 1H), 3.55 (dd, J=10.4, 9.5 Hz, 1H), 1.35 (s, 3H), 1.30 (s, 3H); ¹³C NMR (150 MHz, CD₃CN): δ 159.5, 150.1, 144.2 (q, J=6.3 Hz), 123.5 (q, J=266.4 Hz), 106.3 (q, J=32.9 Hz), 99.9, 96.3 (d, J=210.9 Hz), 73.9 (d, J=3.8 Hz), 70.5 (d, J=24.5 Hz), 65.4, 63.0, 29.1, 19.8; ¹⁹F NMR (470 MHz, CD₃CN): δ −64.1, −168.0. HRMS (EI⁺) calcd for C₁₃H₁₇F₄N2NaO₆ [M+Na]⁺ 395.0837; found 395.0836.

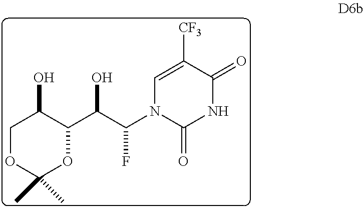

Data for syn-diol, anti-fluorohydrin D6b: [α]_{D}²⁰=−37.2 (c 1.1 in CH₂Cl₂); IR (neat): ν=3424, 1703, 1466, 1379, 1281, 1138, 1042 cm⁻¹; ¹H NMR (600 MHz, CD₃CN): δ 8.26 (s, 1H), 6.67 (dd, J=43.0, 4.9 Hz, 1H), 4.34 (m, 1H), 3.78 (dd, J=11.2, 5.1 Hz, 1H), 3.72 (m, 2H), 3.54 (dd, J=11.2, 8.3 Hz, 1H), 1.39 (s, 3H), 1.26 (s, 3H); ¹³C NMR (150 MHz, CD₃CN): δ 159.5, 150.6, 144.2, 123.6 (q, J=272.9 Hz), 105.9 (q, J=32.5 Hz), 100.0, 92.5 (d, J=206.1 Hz), 74.2 (d, J=4.4 Hz), 72.3 (d, J=27.7 Hz), 65.4, 64.8, 29.0, 19.7; ⁹F NMR (470 MHz, CD₃CN): δ −64.1, −161.7. HRMS (EI⁺) calcd for C₁₃H₁₇F₄N₂NaO₆, [M+Na]+395.0837; found 395.0838.

Cyclization of Diols D6a and D6b

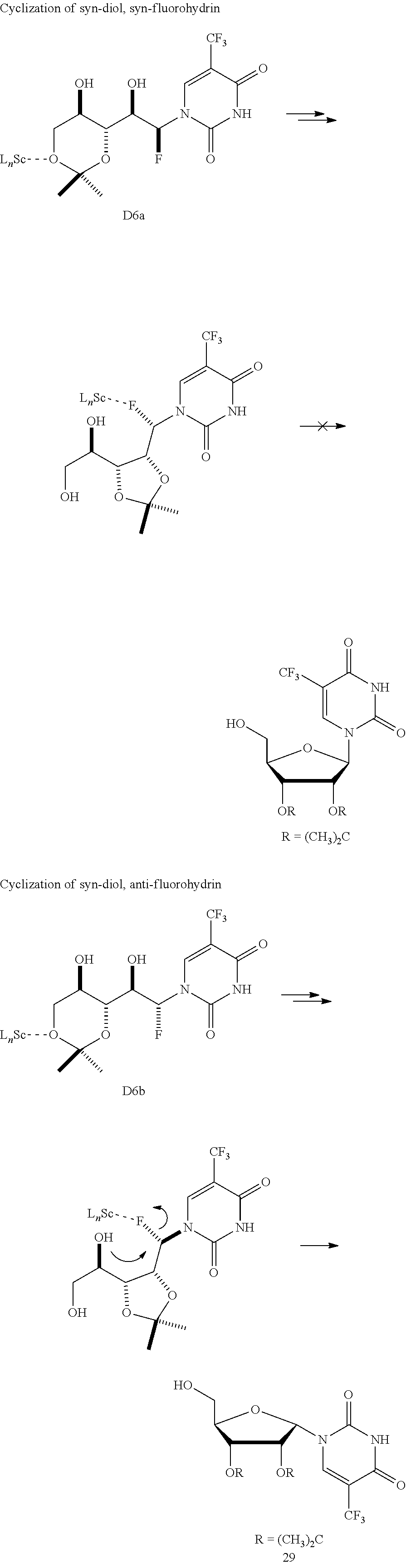

Following General Procedure D, diol D6b was cyclized separately to 29 while diol D6a did not cyclize. This suggests the product from generated from the diol mixture comes only from the D6b diol via an S_{N}2 cyclization.

Following General Procedure D, a solution of D6a and D6b (0.045 g, 0.121 mmol, d.r. (syn/anti)=1:2) and Sc(OTf)₃ (8.9 mg, 0.018 mmol, 0.15 equiv.) was stirred for 24 hours in dry MeCN (1.21 mL). Purification of the crude 29 by flash chromatography (pentane:ethyl acetate—3:7) afforded nucleoside 29 (0.013 g, 45% yield (from anti-fluorohydrin D6b)) as a colorless oil.

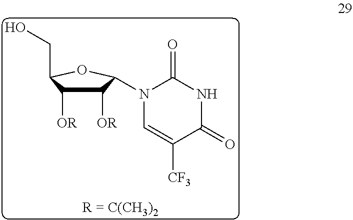

Data for nucleoside analogue 29: [α]_{D}²⁰=−16.7 (c 0.49 in CH₂Cl₂); IR (neat): ν=3405, 2924, 2854, 1702, 1465, 1276 cm⁻¹; ¹H NMR (600 MHz, CD₃CN): δ 9.33 (br s, 1H), 7.97 (q, J=1.2 Hz, 1H), 6.18 (d, J=4.1 Hz, 1H), 4.86 (m, 2H), 4.42 (dd, J=3.6, 2.4 Hz, 1H), 3.67 (m, 2H), 3.21 (dd, J=5.6, 4.4 Hz, 1H), 1.36 (s, 3H), 1.30 (s, 3H); ¹³C NMR (150 MHz, CD₃CN): δ 159.4, 149.9, 143.6 (q, J=6.0 Hz), 123.6 (q, J=269.7 Hz), 113.6, 103.4 (q, J=33.2 Hz), 87.7, 84.7, 82.8, 80.2, 64.0, 25.7, 24.0; ¹⁹F NMR (470 MHz, CD₃CN): δ −63.8 HRMS (EI⁺) calcd for C₁₃H₁₆F₃N₂O₆[M+H]⁺ 353.0955; found 353.0971

Determination of Relative Stereochemistry for Nucleoside 29

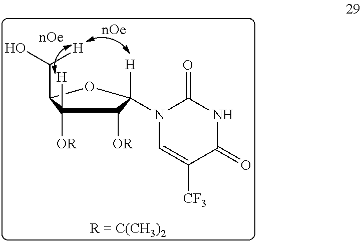

Analysis of 2D NOESY of nucleoside 29 supported the indicated stereochemistry.

Determination of Relative Stereochemistry for Diols D6a and D6b

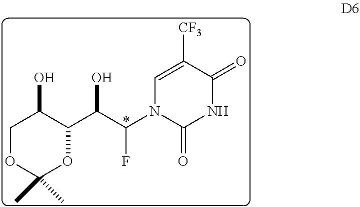

Based on J-based configurational analysis of compounds D5a/D5b, D8a/D8b and XRD analysis of compounds 18a, D7b, D9a a clear trend was established between the stereochemistry at the fluoromethine center and the chemical shift of the fluoromethine proton (*). In every case, the syn-fluorohydrin diol has a lower chemical shift than the diastereomeric anti-fluorohydrin diol. Here, D6a has a chemical shift of 6.33 ppm while D6b has a chemical shift of 6.67 ppm for the fluoromethine proton. D6a was assigned as the syn-fluorohydrin diol and D6b the anti-fluorohydrin diol.

Determination of Enantiomeric Excess of Nucleoside 29

Following General Procedures A, B, and C using a 1:1 mixture of L-:D-proline, a racemic sample of nucleoside 29 was prepared. The enantiomeric nucleosides were separated by chiral HPLC using a a Lux® 3 μm Amylose-1 column; flow rate 0.25 mL/min; eluent: hexanes-iPrOH 90:10; detection at 254 nm; retention time=9.10 min for (+)-29; 13.14 min for (−)-29. The enantiomeric ratio of the optically enriched (−)-29 nucleoside was determined using the same method (94:6 e.r.).

Preparation of S7, Hydrate SM7, Aldol Adduct A7, Diol Adducts D7a and D7b, and Nucleoside Analogue 30

α-Fluorination/Aldol and Syn-Reduction of Syn- and Anti-Fluorohydrins A

Following General Procedure A, a solution of phthalimidoacetaldehyde (0.100 g, 0.529 mmol, 1.5 equiv.), NFSI (0.167 g, 0.529 mmol, 1.5 equiv.), L-proline (0.061 g, 0.529 mmol, 1.5 equiv.) and 2,6-lutidine (0.061 mL, 0.529 mmol, 1.5 equiv.) was stirred for 12 hours at 4° C. in DMF (0.71 mL). Dioxanone 8 (0.042 mL, 0.353 mmol, 1 equiv.) in CH₂Cl₂ (0.88 mL) was then added and the reaction mixture was stirred for 48 hrs at room temperature. Purification of the crude fluorohydrin A7 by flash chromatography (pentane:ethyl acetate—1:1) afforded fluorohydrin A7 (0.069 g, 58% yield, d.r. 2.2:1) as a yellow oil. Following General Procedure B, Me₄NHB(OAc)₃ (0.776 g, 2.95 mmol) and AcOH (0.337 mL, 5.90 mmol) were added to a stirred solution of A7 (0.200 g, 0.59 mmol) at −15° C. in MeCN (5.90 mL) and the reaction mixture was stirred for 24 hrs. Purification of the crude diols D7a and D7b by flash chromatography (pentane:ethyl acetate—3:7) afforded diols D7a and D7b (0.094 g, 47% yield, d.r. (syn/anti)=1.5:1) as white solids.

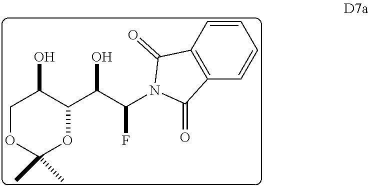

Data for syn-diol, syn-fluorohydrin D7a: [α]_{D}²⁰=−11.4 (c 2.0 in CH₂Cl₂); IR (neat): ν=3442, 2992, 1785, 1724, 1377, 1074, 721 cm⁻¹; ¹H NMR (600 MHz, CD₃CN): δ 7.93 (m, 2H), 7.89 (m, 2H), 6.07 (dd, J=48.6, 7.9 Hz, 1H), 4.76 (m, 1H), 4.43 (m, 1H), 3.73 (m, 2H), 3.58 (dd, J=8.8, 6.0 Hz, 1H), 3.47 (m, 1H), 3.41 (m, 1H), 1.21 (s, 3H), 0.92 (s, 3H); ¹³C NMR (150 MHz, CD₃CN): δ 167.8 (d, J=1.5 Hz), 136.0, 132.5, 124.6, 99.1, 91.1 (d, J=202.0 Hz), 73.3 (d, J=6.6 Hz), 71.8 (d, J=25.3 Hz), 65.1, 64.5, 28.1, 19.3; ¹⁹F NMR (470 MHz, CD₃CN): δ −157.8 HRMS (EI⁺) calcd for C₁₆H₁₉FNO₆ [M+H]⁺ 340.1191; found 340.1190.

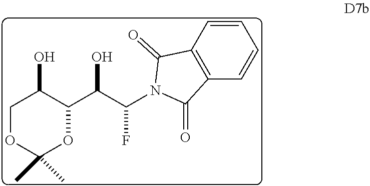

Data for syn-diol, anti-fluorohydrin D7b: [α]_{D}²⁰=−1.0 (c 2.3 in CH₂Cl₂); IR (neat): ν=3442, 2992, 1784, 1725, 1375, 1070, 723 cm⁻¹; ¹H NMR (600 MHz, CD₃CN): δ 7.94 (m, 2H), 7.89 (m, 2H), 6.34 (dd, J=46.0, 9.2 Hz, 1H), 4.80 (m, 1H), 3.92 (ddd, J=9.5, 1.8, 1.4 Hz, 1H), 3.84 (m, 2H), 3.73 (m, 1H), 3.60 (dd, J=10.8, 8.7 Hz, 1H), 3.30 (m, 1H), 1.47 (s, 3H), 1.35 (s, 3H); ¹³C NMR (150 MHz, CD₃CN): δ 168.1 (d, J=1.6 Hz), 136.0, 132.3, 124.6, 99.4, 89.5 (d, J=202.4 Hz), 75.1, 68.7 (d, J=31.7 Hz), 65.3, 63.1 (d, J=3.1 Hz), 28.6, 19.5; ¹⁹F NMR (470 MHz, CDCl₃): δ −159.8. HRMS (EI⁺) calcd for C₁₆H₁₉FNO₆ [M+H]⁺ 340.1191; found 340.1172

Cyclization of Diols D7a and D7b

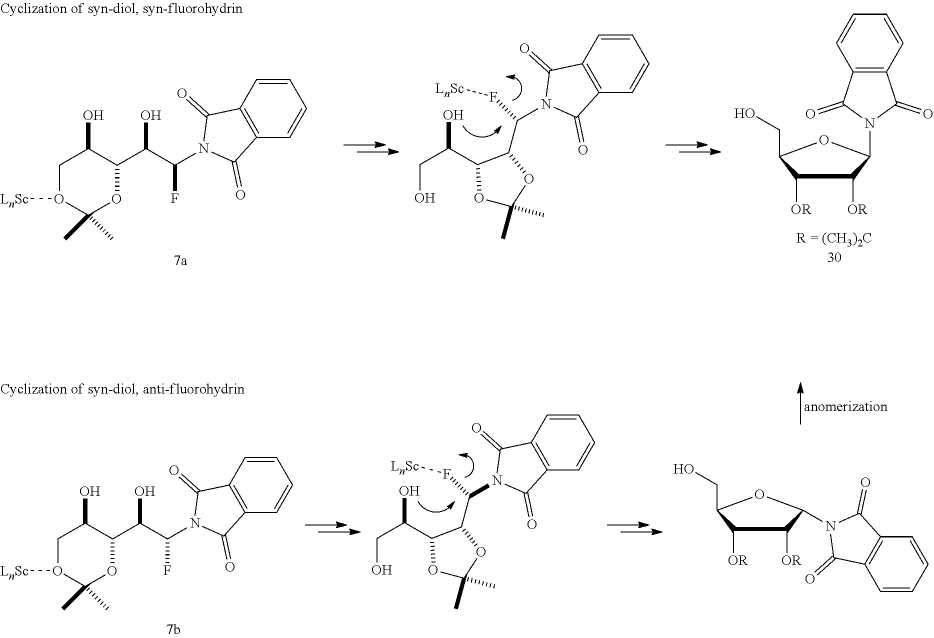

Following General Procedure D, diol D7a was cyclized separately to 30 while diol D7b cyclized to a mixture of 30 and its corresponding α-anomer. The diol mixture comes from both diols via an S_{N}2 cyclization and some epimerization of the α-anomer. Such emperizations have been reported for nucleosides (31).

Following General Procedure D, a solution of D7a and D7b (0.033 g, 0.097 mmol, 1.0 equiv., d.r. (syn/anti)=2:1) and Sc(OTf)₃ (0.120 g, 0.243 mmol, 2.5 equiv.) was stirred for 6 hours in MeCN (0.65 mL). 0.25 mL of pyridine and 0.25 mL of acetic anhydride were added and the reaction mixture was allowed to stir for a further 1.5 hrs. Purification of the crude 30 by flash chromatography (pentane:ethyl acetate—7:3) afforded nucleoside analogue 30 (0.027 g, 69% yield) as a colourless oil.

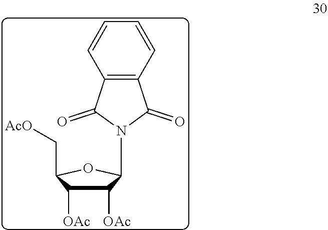

Data for nucleoside analogue 30: [α]_{D}²⁰=−9.0 (c 1.96 in CH₂Cl₂); IR (neat): ν=2922, 1781, 1744, 1721, 1374, 1222, 1047, 720 cm⁻¹; ¹H NMR (500 MHz, CDCl₃): δ 7.88 (m, 2H), 7.77 (m, 2H), 5.94 (dd, J=6.0, 4.1 Hz, 1H), 5.87 (d, J=4.1 Hz, 1H), 5.65 (dd, J=6.1, 6.0 Hz, 1H), 4.49 (dd, J=12.1, 3.4 Hz, 1H), 4.29 (ddd, J=9.5, 5.9, 3.4 Hz, 1H), 4.21 (dd, J=12.1, 5.9, 1H), 2.12 (s, 3H), 2.11 (s, 3H), 2.09 (s, 3H); ¹³C NMR (150 MHz, CDCl₃): δ 170.9, 169.8, 169.7, 166.9, 134.8, 131.7, 124.0, 82.8, 79.2, 72.0, 70.6, 63.2, 20.9, 20.7, 20.7. HRMS (EI⁺) calcd for C₁₉H₂₃N₂O₉ [M+NH₄]⁺ 423.1398; found 423.1378

Determination of Relative Stereochemistry for Diol D7b

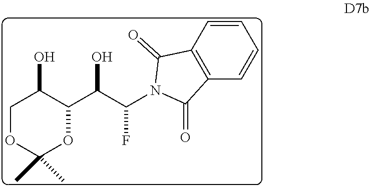

Recrystallization in ethanol allowed for the relative stereochemistry to be assigned using single X-ray crystallography.

Determination of the Relative Stereochemistry for Nucleoside 30

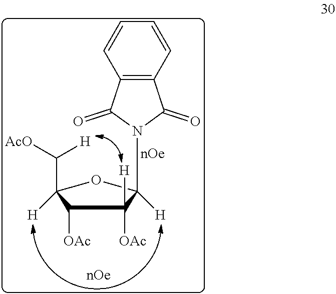

Analysis of 2D NOESY of nucleoside 30 supported the indicated stereochemistry.

Determination of Enantiomeric Excess of Diol Ent-D7a

Following General Procedures A and B, using a 1:1 mixture of L-:D-proline, a racemic sample of diol D7a was prepared. The enantiomeric nucleosides were separated by chiral HPLC using a a Lux® 3 μm Amylose-1 column; flow rate 0.25 mL/min; eluent: hexanes-iPrOH 90:10; detection at 254 nm; retention time=9.10 min for (−)-D7a; 13.14 min for (+)-D7a. The enantiomeric ratio of the optically enriched (+)-D7a diol was determined using the same method (95:5 e.r.).

Preparation of SM8, Aldol Adduct A8, Diol Adducts D8a/D8b, and Nucleoside Analogues 32/33

A solution of deazadenine (0.500 g, 1.79 mmol, 1.0 equiv.), bromoacetaldehyde diethyl acetal (0.323 mL, 2.15 mmol, 1.25 equiv.) and K₂CO₃ (0.491 g, 3.58 mmol, 2.0 equiv.) was stirred for 24 hours at 90° C. in DMF (9.00 mL). The reaction mixture was then filtered and washed with 10 mL of CH₂Cl₂ and concentrated under reduced pressure. Purification of crude S8 by flash chromatography (pentane:ethyl acetate—7:3) afforded S8 (0.375 g, 53% yield) as a white solid. A solution of S8 (17.0 g, 43.0 mmol, 1.0 equiv.) was heated to 70° C. in 2.0 M HCl (129 mL, 258 mmol, 6.0 equiv.) for 1 hours. The reaction mixture was then cooled to room temperature and allowed to stir for a further 2 hrs. The reaction mixture was stored overnight at −20° C. and the formed precipitate was then filtered and washed with 1:1 dioxane:water (10 mL×2). The filtrate SM8 was dried under reduced pressure and the resulting product SM8 (7.88 g, 54% yield) was used in the reaction without purification.

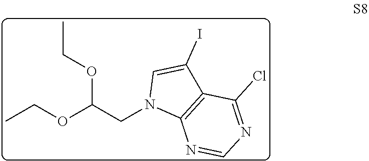 [SMILES: CCOC(Cn1cc(I)c2c(Cl)ncnc21)OCC]

Data for S8: ¹H NMR (600 MHz, CDCl₃): δ 8.61 (s, 1H), 7.50 (s, 1H), 4.67 (t, J=5.1 Hz, 1H), 4.35 (d, J=5.1 Hz, 2H), 3.73 (m, 2H), 3.48 (m, 2H), 1.16 (m, 6H); ¹³C NMR (150 MHz, CDCl₃): δ 152.7, 151.1, 150.8, 136.3, 116.9, 100.7, 63.9, 50.6, 47.7, 15.3. HRMS (EI⁺) calcd for C₁₂H₁₆CIlN₃O₂[M+H]⁺ 395.9970; found 395.9973

α-Fluorination/Aldol

Following General Procedure A, a solution of SM8 (2.00 g, 5.86 mmol, 1 equiv.), NFSI (1.85 g, 5.86 mmol, 1.0 equiv.), L-proline (0.674 g, 5.86 mmol, 1.0 equiv.) and NaHCO₃ (0.984 g, 11.71 mmol, 2.0 equiv.) was stirred for 18 hours at 20° C. in DMF (10 mL). Dioxanone 8 (0.762 g, 5.86 mmol, 1.0 equiv.) was then added and the reaction mixture was stirred for 36 hrs at room temperature. Purification of the crude A8 by flash chromatography (25-75% ethyl acetate in pentane) afforded syn- and anti-fluorohydrins A8 (1.58 g, 57% yield, d.r. 1.2:1) as a light yellow solid.

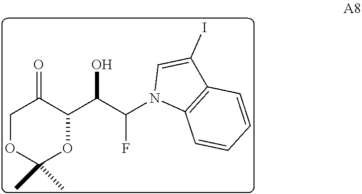

Data for syn- and anti-fluorohydrins A8: IR (neat): ν=3145, 2988, 1747, 1575, 1539, 1444, 1205, 1084, 949, 734 cm⁻¹; ¹H NMR (600 MHz, dmso-d₆): δ 8.76, 8.74, 8.39, 8.24, 6.89, 6.85, 6.37, 6.12, 4.98, 4.76, 4.61, 4.32, 4.30, 4.05, 3.95, 3.93, 1.40, 1.34, 1.33, 1.31 ¹³C NMR (150 MHz, dmso-d₆): δ 206.3, 206.1, 151.6, 151.5, 151.3, 151.2, 151.0, 134.5, 134.1, 116.8, 116.7, 100.4, 100.1, 91.4, 09.4, 76.1, 74.7, 68.7, 68.0, 66.6, 66.4, 55.3, 55.1, 24.6, 24.1, 22.9, 22.7 ¹⁹F NMR (470 MHz, dmso-d₆): δ −146.0, −152.6. HRMS (EI⁺) calcd for C₁₄H₁₅ClFIN₃O₄[M+H]⁺ 469.9774; found 469.9779

Syn-Reduction of Syn- and Anti-Fluorohydrins A8

Following General Procedure B, NaHB(OAc)₃ (0.316 g, 1.49 mmol, 5 equiv.) and AcOH (0.171 mL, 2.98 mmol, 10 equiv.) were added to a stirred solution of A8 (0.140 g, 0.298 mmol, 1 equiv.) at 0° C. in MeCN (2.8 mL). The reaction mixture was then stirred at room temperature for 2 hrs. Purification of the crude diols D8a and D8b by flash chromatography (pentane:ethyl acetate −70:30) afforded diols D8a and D8b (0.141 g, 77% yield, d.r. (syn/anti)=1.5:1) as a white solid.

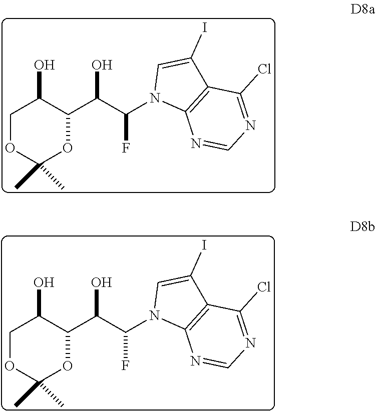

Data for syn-diol, syn-fluorohydrin D8a: [α]_{D}²⁰=−19.6 (c 2.0 in CH₂Cl₂); IR (neat): ν=3335, 2989, 2890, 1577, 1540, 1445, 1206, 1076, 951 cm⁻¹; ¹H NMR (600 MHz, dmso-d₆): δ 8.73 (s, 1H), 8.27 (s, 1H), 6.73 (dd, J=49.4, 7.0 Hz, 1H), 6.08 (br s, 1H), 4.84 (d, J=4.1 Hz, 1H), 4.59 (m, 1H), 3.59 (m, 1H), 3.44 (m, 1H), 3.42 (m, 1H), 3.33 (m, 1H), 1.16 (s, 3H), 1.13 (s, 3H); ¹³C NMR (150 MHz, dmso-d₆): δ 151.4, 151.2, 151.1, 134.5, 116.7, 97.8, 92.0 (d, J=203.3), 73.2 (d, J=5.7 Hz), 71.0 (d, J=24.2 Hz), 63.8, 62.5, 54.9, 28.0, 19.1; ¹⁹F NMR (470 MHz, dmso-d₆): δ −147.1. HRMS (EI⁺) calcd for C₁₄H₁₅ClFIN₃O₄[M+H]⁺ 471.9931; found 471.9940.

Data for syn-diol, anti-fluorohydrin D8b: [α]_{D}²⁰=−11.6 (c 0.38 in CH₂Cl₂); IR (neat): ν=3363, 2931, 2890, 1579, 1540, 1444, 1212, 1067, 951 cm⁻¹; ¹H NMR (600 MHz, dmso-d₆): δ 8.73 (s, 1H), 8.34 (s, 1H), 6.97 (dd, J=46.9, 7.9 Hz, 1H), 5.74 (d, J=5.7 Hz, 1H), 5.22 (d, J=5.7 Hz, 1H), 4.61 (m, 1H), 3.84 (m, 1H), 3.72 (m, 1H), 3.52 (dd, J=11.7, 8.7 Hz, 1H), 1.35 (s, 3H), 1.20 (s, 3H); ¹³C NMR (150 MHz, dmso-d₆): δ 151.5, 151.4, 151.2, 134.1, 116.6, 97.9, 90.9 (d, J=203.5 Hz), 74.3, 69.1 (d, J=30.3 Hz), 64.2, 61.4, 54.8, 28.4, 19.0; ¹⁹F NMR (470 MHz, dmso-d₆): δ −146.3. HRMS (EI⁺) calcd for C₁₄H₁₅ClFIN₃O₄[M+H]⁺ 471.9931; found 471.9940

Cyclization of Diol D8a

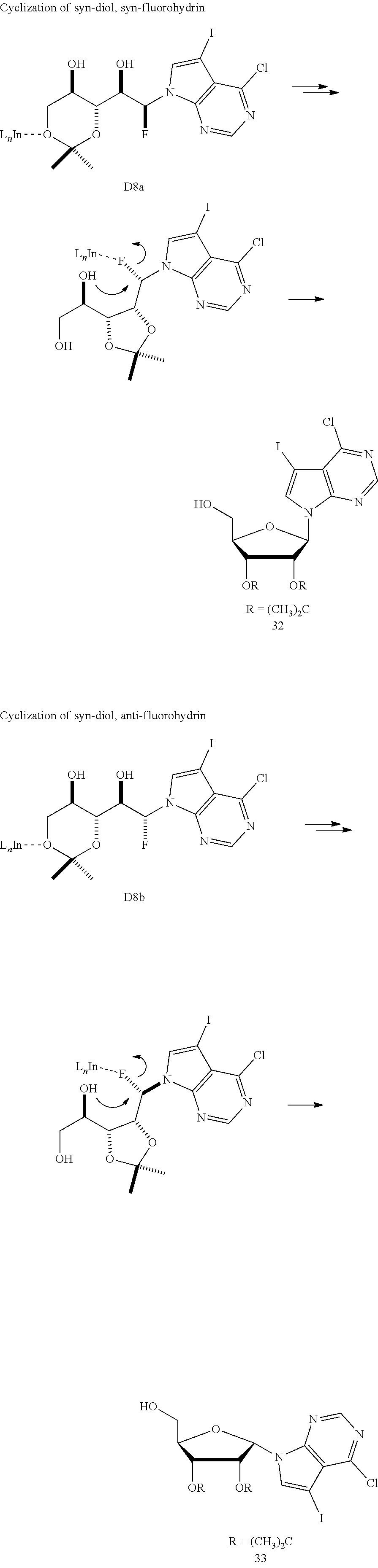

Following General Procedure D, diol D8a was cyclized separately to 32 while diol D8b cyclized to 33. This supports an S_{N}2 cyclization without subsequent epimerization.

Following General Procedure D, a solution of D8a (0.050 g, 0.106 mmol, 1.0 equiv.) and InCl₃ (2.3 mg, 0.011 mmol, 0.10 equiv.) was stirred for 16 hrs in dry MeCN (1.00 mL). Purification of the crude nucleoside 32 by flash chromatography (20-80% ethyl acetate in pentanes) afforded nucleoside 32 (0.029 g, 61% yield) as a white solid.

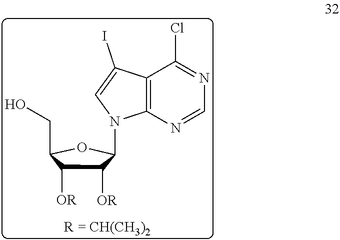

Data for nucleoside analogue 32: [α]_{D}²⁰=−23.9 (c 0.46 in CH₂Cl₂); IR (neat): ν=3339, 3113, 2935, 1576, 1539, 1445, 1207, 1108, 951 cm⁻¹; ¹H NMR (600 MHz, dmso-d₆): 6 8.69 (s, 1H), 8.23 (s, 1H), 6.34 (d, J=3.1 Hz, 1H), 5.19 (dd, J=6.3, 3.1 Hz, 1H), 5.14 (br s, 1H), 4.94 (dd, J=6.3, 2.9 Hz, 1H), 4.20 (m, 1H), 3.56 (m, 2H), 1.54 (s, 3H), 1.31 (s, 3H); ¹³C NMR (150 MHz, dmso-d₆): δ 151.2, 150.8, 150.4, 133.9, 116.7, 113.2, 89.4, 86.3, 83.9, 80.9, 61.4, 53.7, 27.0, 25.1. HRMS (EI⁺) calcd for C₁₄H₁₆CIlN₃O₄[M+H]⁺ 451.9869; found 451.9875

Cyclization of Diol D8b

Following General Procedure D, a solution of D8b (0.050 g, 0.106 mmol, 1.0 equiv.) and InCl₃ (2.3 mg, 0.011 mmol, 0.10 equiv.) was stirred for 16 hrs in dry MeCN (1.00 mL). Purification of the crude nucleoside 33 by flash chromatography (20-80% ethyl acetate in pentanes) afforded nucleoside 33 (0.034 g, 70% yield) as a white solid.

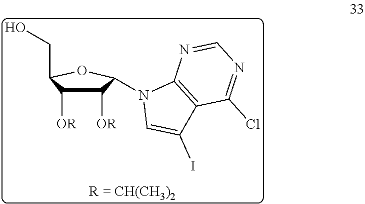

Data for nucleoside analogue 33: [α]_{D}²⁰=−47.8 (c 0.51 in CHCl₃); ¹H NMR (600 MHz, dmso-d₆): δ 8.66 (s, 1H), 7.81 (s, 1H), 6.73 (d, J=4.3 Hz, 1H), 5.22 (br s, 1H), 4.91 (m, 2H), 4.41 (dd, J=3.6, 3.1 Hz, 1H), 3.62 (m, 2H), 1.32 (s, 3H), 1.23 (s, 3H); ¹³C NMR (150 MHz, dmso-d₆): δ 151.0, 150.7, 149.8, 134.6, 116.3, 112.3, 85.6, 83.1, 81.9, 79.4, 62.5, 51.9, 25.2, 23.9. HRMS (EI⁺) calcd for C₁₄H₁₆CIlN₃O₄[M+H]⁺ 451.9869; found 451.9888

Determination of Relative Stereochemistry for Diol D8a

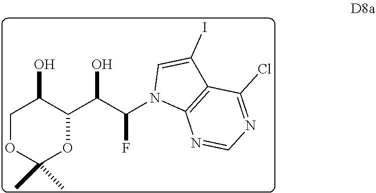

The relative stereochemistry of diol D8a was determined by J-based configurational analysis. See J-based configurational analysis section for details.

Determination of Relative Stereochemistry for Diol D8b

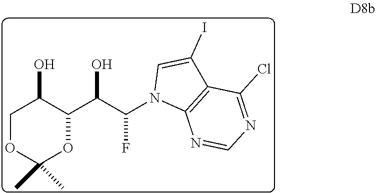

The relative stereochemistry of diol D8b was determined by J-based configurational analysis. See J-based configurational analysis section for details

Determination of Relative Stereochemistry for Nucleoside 32

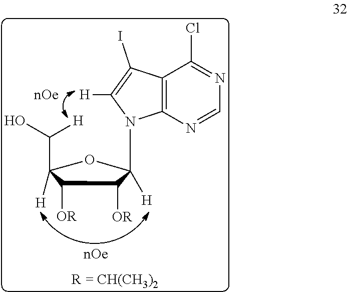

Analysis of 2D NOESY of nucleoside 32 supported the indicated stereochemistry.

Determination of Relative Stereochemistry for Nucleoside 33

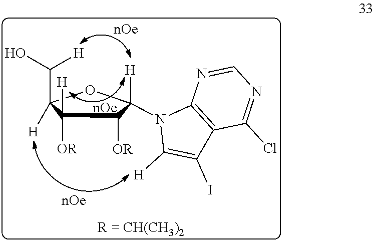

Analysis of 2D NOESY of nucleoside 33 supported the indicated stereochemistry.

Determination of Enantiomeric Excess of Diol D8a

Following General Procedures A and B, using a 1:1 mixture of L-:D-proline, a racemic sample of diol D8a was prepared. The enantiomeric diols were separated by chiral HPLC using an IB column; eluent: 90:10 (MeCN:water) to 10:90 (MeCN:water); detection at 230 nm; retention time=12.23 min for (+)-D8a; 13.39 min for (−)-D8a. The enantiomeric ratio of the optically enriched ent-D8a diol was determined using the same method (90:10 e.r.).

Determination of Enantiomeric Excess of Diol D8b

Following General Procedures A and B, using a 1:1 mixture of L-:D-proline, a racemic sample of diol D8b was prepared. The enantiomeric diols were separated by chiral HPLC using a IG column; eluent: 90:10 (MeCN:water) to 10:90 (MeCN:water); detection at 230 nm; retention time=12.35 min for (−)-D8b; 12.56 min for (+)-D8b. The enantiomeric ratio of the optically enriched ent-D8b diol was determined using the same method (93:7 e.r.).

Preparation of SM9, Aldehyde S9, Aldol Adduct A9, Diol Adducts D9a/D9b, and Nucleoside Analogues S19/NA9

A solution of iodouracil (2.50 g, 10.5 mmol, 1.0 equiv.), bromoacetaldehyde diethyl acetal (1.91 mL, 12.7 mmol, 1.2 equiv.) and K₂CO₃ (2.92 g, 21.1 mmol, 2.0 equiv.) was stirred for 16 hours at 90° C. in DMF (70 mL). The reaction mixture was filtered, and the filtrate was diluted with 200 mL of ethyl acetate. The organic layer was washed 3 times with water, separated, dried over MgSO₄, filtered, and concentrated under reduced pressure. Purification of crude S9 by flash chromatography (pentane:ethyl acetate—75:25) afforded S9 (0.301 g, 8% yield) as a white solid. A solution of S9 (0.142 g, 0.401 mmol, 1.0 equiv.) was heated to 90° C. in 0.5 M HCl (0.40 mL) for 5 hours. Upon complete conversion to aldehyde/hydrate SM9, the reaction mixture was concentrated under reduced pressure and the resulting aldehyde/hydrate SM9 was used in the reaction without purification.

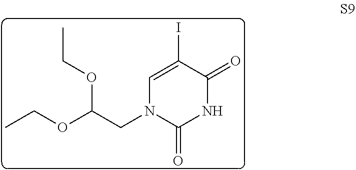 [SMILES: CCOC(Cn1cc(I)c(=O)[nH]c1=O)OCC]

Data for S9: IR (neat): ν=2975, 1686, 1439, 1121, 1059, 1021 cm⁻¹; 1H NMR (600 MHz, CDCl₃): δ 8.56 (br s, 1H), 7.82 (s, 1H), 4.61 (t, J=5.0 Hz), 3.88 (d, J=5.0 Hz), 3.78 (m, 2H), 3.54 (m, 2H), 1.21 (m, 6H); ¹³C NMR (150 MHz, CDCl₃): δ 158.6, 150.0, 147.0 (q, J=5.8 Hz), 121.9 (q, J=270.5 Hz), 104.7 (q, J=33.5 Hz), 100.0, 64.6, 51.0, 15.3. HRMS (EI⁺) calcd for C₁₀H₁₆1N₂O₄[M+H]⁺ 355.0149; found 355.0145

α-Fluorination/Aldol and Syn-Reduction of Syn- and Anti-Fluorohydrins A9

Following General Procedure A, a solution of S9 (0.401 mmol), NFSI (0.126 g, 0.401 mmol), L-proline (0.046 g, 0.401 mmol) and NaHCO₃ (0.034 g, 0.401 mmol) was stirred for 12 hours at 4° C. in DMF (0.53 mL). Dioxanone 8 (0.053 mL, 0.270 mmol) in CH₂Cl₂ (0.67 mL) was then added and the reaction mixture was stirred for 72 hrs at 4° C. Purification of the crude fluorohydrin A9 by flash chromatography (pentane-ethyl acetate—1:1) afforded fluorohydrin A9. as a yellow oil. Following General Procedure B, Me₄NHB(OAc)₃ (0.066 g, 0.251 mmol) and AcOH (0.0.30 mL, 0.502 mmol) were added to a stirred solution of A9 (0.021 g, 0.049 mmol) at −15° C. in MeCN (0.49 mL) and the reaction mixture was stirred for 24 hrs. The crude diols D9a and D9b were used directly for the cyclization owing to challenges with stability and purification.

Cyclization of Diols D9a and D9b

Following General Procedure C, a solution of D9a and D9b (16.2 mg, 0.038 mmol, 1 equiv.) and 2 M NaOH (0.038 mL, 0.38 mmol, 10 equiv.) was stirred for 18 hours in MeCN (1.51 mL). Purification of the crude nucleoside S19 by flash chromatography (CH₂Cl₂:MeOH—90:10) afforded nucleoside S19 as a white solid. S19 (10.3 mg, 0.025 mmol) was dissolved in MeOD (0.25 mL) and two drops of 1 M HCl was added and the solution was left for 12 hrs at room temperature. Subsequently, the reaction mixture was concentrated under reduced pressure to afford NA9 as a white solid. The spectral data matched previous reports (37).

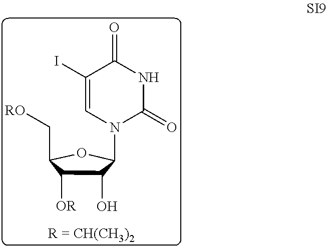

Data for nucleoside analogue S19: ¹H NMR (600 MHz, MeOD): δ 7.99 (s, 1H), 5.58 (s, 1H), 4.35 (d, J=4.5 Hz, 1H), 4.19 (dd, J=10.0, 4.6 Hz, 1H), 4.08 (dd, J=10.0, 9.7 Hz, 1H), 3.83 (m, 2H), 1.57 (s, 3H), 1.45 (s, 3H); ¹³C NMR (150 MHz, MeOD): δ 162.8, 151.7, 147.2, 102.5, 95.7, 74.5, 73.8, 72.5, 68.9, 65.8, 29.3, 20.0

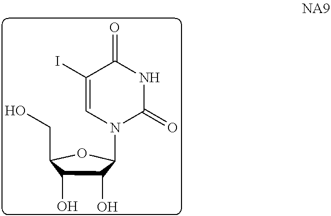

Data for nucleoside NA9: [α]_{D}²⁰=−41 (c=0.1, MeOH); IR (neat): ν=3353, 2929, 1679, 1447, 1262, 1101, 1023, 799 cm⁻¹; ¹H NMR (600 MHz, MeOD): δ 8.61 (s, 1H), 5.86 (d, J=3.6 Hz, 1H), 4.16-4.17 (m, 2H), 4.02-4.03 (m, 1H), 3.89 (dd, J=12.2, 2.6 Hz, 1H), 3.76 (dd, J=12.1, 2.5 Hz, 1H); ¹³C NMR (150 MHz, MeOD): δ 162.8, 152.2, 147.3, 90.9, 86.3, 76.1, 70.9, 68.3, 61.7. HRMS (EI⁺) calcd for C₉H₁₂IN₂O₆[M+H]⁺ 370.9735; found: 370.9739

Determination of Relative Stereochemistry for Diols D9a

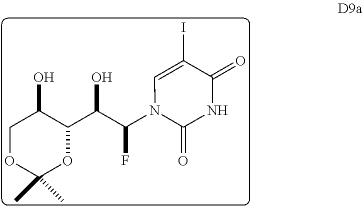

Recrystallization in ethanol allowed for the relative stereochemistry to be assigned using single X-ray crystallography.

Preparation of Nucleoside Analogue 36

To a solution of nucleoside analogue 17 (0.020 g, 0.083 mmol, 1.0 equiv.) in dry CH₂Cl₂ (0.83 mL) was added TEMPO (1.3 mg, 0.008 mmol, 0.10 equiv.) and (diacetoxyiodo)benzene (0.067 g, 0.208 mmol, 2.5 equiv.). Following 18 hrs or complete consumption of 17 as monitored by ¹H NMR spectroscopy, the reaction mixture was cooled to room temperature and diluted with CH₂Cl₂. The organic layer was then washed with saturated sodium bicarbonate solution, dried over MgSO₄, filtered, and concentrated under reduced pressure to yield crude 36. Purification of the crude nucleoside 36 by flash chromatography (pentane:ethyl acetate—1:1) afforded nucleoside 36 (0.019 g, 92% yield) as a white solid.

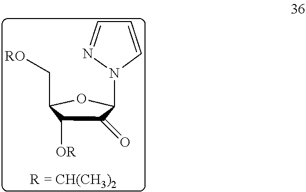

Data for nucleoside analogue 36: [α]_{D}²⁰=−115.6 (c 1.0 in MeCN); IR (neat): ν=3001, 2989, 1694, 1374, 1305, 1088 cm⁻¹; ¹H NMR (600 MHz, CD₃CN): δ 7.80 (d, J=2.4 Hz, 1H), 7.62 (d, J=1.5 Hz, 1H), 6.36 (dd, J=2.4, 1.5 Hz, 1H), 5.78 (s, 1H), 4.69 (d, J=11.1 Hz, 1H), 4.22 (d, J=10.0, 5.0 Hz, 1H), 4.13 (dd, J=10.6, 10.6 Hz, 1H), 3.87 (ddd, J=11.1, 10.0, 5.0 Hz, 1H), 1.56 (s, 3H), 1.45 (s, 3H); ¹³C NMR (150 MHz, CD₃CN): δ 201.5, 143.3, 133.2, 108.1, 103.5, 86.5, 76.8, 69.4, 66.1, 29.3, 20.0. HRMS (EI⁺) calcd for C₁₁H₁₇N₂O₅ [M+H]⁺ 257.1132; found 257.1130

Determination of Relative Stereochemistry for Nucleoside 36

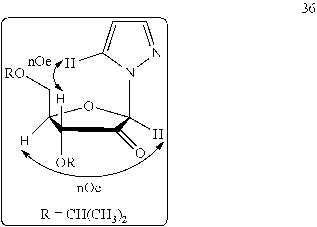

Analysis of 2D NOESY of nucleoside 36 supported the indicated stereochemistry

Preparation of Nucleoside Analogue 37

To a solution of nucleoside analogue 35 (0.100 g, 0.352 mmol, 1 equiv.) in THF (3.52 mL) was added 1, 1′-thiocarbonyldiimidazole (0.125 g, 0.704 mmol, 2 equiv.). The reaction mixture was stirred for 24 hrs. Subsequently, CH₂Cl₂ (10 mL) was added to the reaction mixture and washed with water 3 times. The organic layer was dried over MgSO₄, filtered, and concentrated under reduced pressure to yield crude S37. Purification of crude S37 by flash chromatography (ethyl acetate) afforded S37 (0.129 g, 96%).

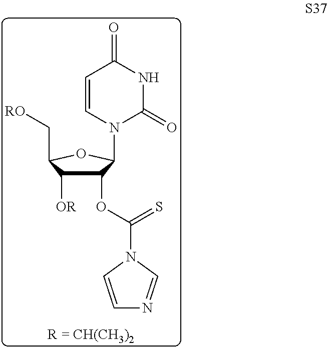

Data for nucleoside analogue S37: [α]_{D}²⁰=+25.8 (c 1.2 in MeCN); IR (neat): ν=3000, 1701, 1443, 1375, 1039, 918, 749 cm⁻¹; ¹H NMR (600 MHz, CD₃CN): δ 9.34 (br s, 1H), 8.38 (s, 1H), 7.73 (s, 1H), 7.43 (d, J=7.4 Hz, 1H), 7.04 (s, 1H), 6.08 (d, J=5.2 Hz, 1H), 5.88 (d, J=5.2 Hz, 1H), 5.69 (d, J=7.4 Hz, 1H), 4.22 (m, 2H), 4.06 (dd, J=10.4 Hz, 1H), 3.83 (ddd, J=10.4, 10.3, 5.0 Hz, 1H), 1.55 (s, 3H), 1.39 (s, 3H); ¹³C NMR (150 MHz, CD₃CN): δ 184.8, 164.1, 151.3, 143.3, 138.4, 132.3, 119.8, 103.8, 102.9, 92.4, 82.7, 73.5, 72.8, 65.5, 29.5, 20.4. HRMS (EI⁺) calcd for C₁₆H₁₉N₄O₆S [M+H]⁺ 395.1020; found 395.1010

To a solution of nucleoside S37 (0.020 g, 0.045 mmol, 1 equiv.) in dry toluene (3.0 mL) under nitrogen was added tributyltin hydride (0.024 mL, 0.090 mmol, 2 equiv.) and AIBN (1.8 mgs, 0.011 mmol, 0.25 equiv.). The resulting reaction mixture was purged with nitrogen for 30 minutes. Subsequently, the reaction mixture was stirred for 16 hrs at 90° C. The reaction mixture was diluted with CH₂Cl₂ (10 mL). The organic layer was washed with water, separated, dried over MgSO₄, filtered, and concentrated under reduced pressure to yield crude 37. Purification of crude 37 by flash chromatography (ethyl acetate) afforded nucleoside 37 (6.8 mg, 57%) as a colorless oil.

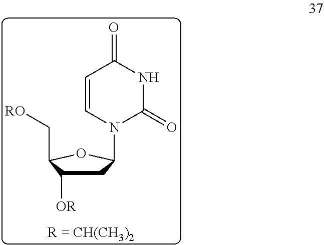

Data for nucleoside analogue 37: [α]_{D}²⁰=+7.8 (c 0.32 in MeOH); ¹H NMR (600 MHz, CD₃CN): δ 8.94 (br s, 1H), 7.50 (d, J=8.2 Hz, 1H), 6.14 (dd, J=8.7, 2.1 Hz, 1H), 5.63 (d, J=8.2 Hz, 1H), 4.10 (dd, J=10.0, 4.6 Hz, 1H), 4.00 (dd, J=10.3, 10.0 Hz, 1H), 3.94 (m, 1H), 3.35 (ddd, J=10.3, 10.0, 4.6 Hz, 1H), 2.27 (m, 1H), 2.17 (m, 1H), 1.52 (s, 3H), 1.37 (s, 3H); ¹³C NMR (150 MHz, CD₃CN): δ 164.1, 151.6, 142.6, 103.3, 102.2, 84.4, 76.3, 72.7, 65.6, 36.4, 29.8, 20.5. HRMS (EI⁺) calcd for C₁₂H₁₇N₂O₅ [M+H]⁺ 269.1132; found 269.1111.

Preparation of Nucleoside Analogue 38

To a stirred solution of nucleoside 36 (0.020 g, 0.084 mmol, 1.0 equiv.) in dry THF (0.84 mL) was added methylmagnesium bromide (0.126 mL, 0.378 mmol, 4.5 equiv.) at −78° C. and the resulting reaction mixture was stirred for 3.5 hrs. The reaction mixture was quenched at −78° C. with 0.50 mL of an ammonium chloride:methanol solution (1:1—saturated ammonium chloride solution:methanol) and warmed to room temperature. The resulting mixture was diluted with 3 mL of CH₂Cl₂ and washed twice with water. The organic layer was dried over MgSO₄, filtered, and concentrated under reduced pressure to give crude 38. Purification of crude 38 by flash chromatography (ethyl acetate:pentane—30:70) afforded nucleoside analogue 38 (19.1 mg, 90%) as a white solid.

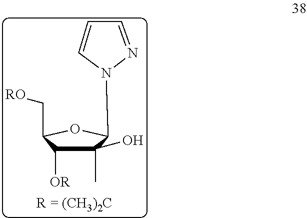

Data for nucleoside analogue 38: [α]_{D}²⁰=−117.7 (c 0.57 in CH₂Cl₂); IR (neat): ν=3425, 2992, 1398, 1384, 1088, 851 cm⁻¹; ¹H NMR (600 MHz, CD₃CN): δ 7.73 (d, J=2.3 Hz, 1H), 7.60 (d, J=1.3 Hz, 1H), 6.33 (dd, J=2.3, 1.3 Hz, 1H), 5.60 (s, 1H), 4.13 (d, J=10.0 Hz, 1H), 4.06 (dd, J=9.8, 4.7 Hz, 1H), 3.93 (dd, J=10.1, 9.8 Hz, 1H), 3.54 (s, 1H), 3.48 (ddd, J=10.1, 10.0, 4.7 Hz, 1H), 1.53 (s, 3H), 1.41 (s, 3H), 1.36 (s, 3H); ¹³C NMR (150 MHz, CD₃CN): 142.1, 132.5, 107.2, 102.2, 95.1, 80.5, 78.4, 71.6, 66.2, 29.7, 20.6, 20.4. HRMS (EI⁺) calcd for C₁₂H₁₉N₂O₄ [M+H]⁺ 255.1339; found 255.1333

Determination of Relative Stereochemistry for Nucleoside 38

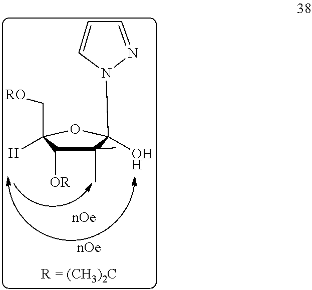

Analysis of 2D NOESY of nucleoside 38 supported the indicated stereochemistry.

Preparation of Nucleoside Analogue 39

To a solution of nucleoside analogue 35 (0.025 g, 0.088 mmol, 1 equiv.) in CH₂Cl₂ (0.45 mL) at 0° C. was added dropwise diethylaminosulfur trifluoride (0.058 mL, 0.44 mmol, 5 equiv.). The reaction mixture was warmed to room temperature and allowed to stir for 1 hr. Subsequently, ethyl acetate (10 mL) was added and the organic layer was washed 3 times with saturated sodium bicarbonate solution. The organic layer was then separated, dried, filtered, and concentrated under reduced pressure. Purification of the crude S39 by flash chromatography (CH₂Cl₂:MeOH 95:5) afforded 2′,2′-anhydrouridine S39 (0.012 g, 51% yield) as a white solid. 2′,2′-anhydrouridine S39 (0.011 g, 0.039 mmol, 1 equiv.) was dissolved in a 1 M HCl:MeOH solution (0.20 mL:0.20 mL). The reaction mixture was heated to 50° C. for 24 hrs and then concentrated under reduced pressure to yield nucleoside 39 (9.5 mg, 100% yield). The spectral data matched previous reports (41).

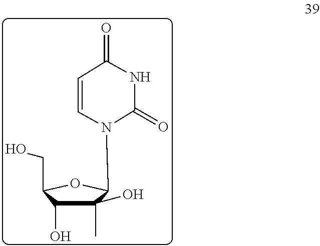

Data for nucleoside analogue 39: ¹H NMR (600 MHz, dmso-d₆): δ 11.28 (d, J=2.1 Hz, 1H), 7.62 (d, J=8.1 Hz, 1H) 5.98 (d, J=4.5 Hz, 1H), 5.56 (dd, J=8.1, 2.1 Hz, 1H), 3.99 (dd, J=4.4, 3.2 Hz, 1H), 3.89 (dd, J=3.6, 3.2 Hz, 1H), 3.73 (ddd, J=5.6, 4.6, 3.6 Hz, 1H), 3.60 (dd, J=11.6, 4.6 Hz, 1H), 3.56 (dd, J=11.6, 5.6 Hz, 1H); ¹³C NMR (150 MHz, dmso-d₆): δ 163.4, 150.5, 142.3, 100.0, 85.1, 84.7, 75.5, 75.1, 60.7. HRMS (EI⁺) calcd for C₉H₁₃N₂O₆ [M+H]⁺ 245.0768; found 245.0777

Preparation of Nucleoside Analogue 43

Methylmagnesium chloride (3.0 M in THF, 1.49 mL, 4.47 mmol, 2.1 equiv.) was added dropwise to a solution of 41 (syn-/anti-fluorohydrin=3:1, 1.00 g, 2.13 mmol, 1.0 equiv.) at −78° C. in CH₂Cl₂ (10 mL). The reaction mixture was stirred at this temperature for 2 hrs and then allowed to warm gradually to room temperature and stirred for 12 hrs. The reaction mixture was quenched with saturated ammonium chloride solution and diluted with ethyl acetate. The organic layer was separated, dried over MgSO₄, filtered, and concentrated under reduced pressure. Purification of crude product 43 by flash chromatography (0-10% MeOH in CH₂Cl₂) afforded nucleoside 43 (0.418 g, 42%) as a white solid.

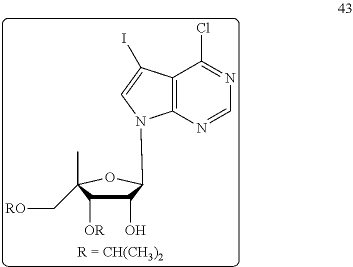

Data for nucleoside analogue 43: [α]_{D}²⁰=−13.6 (c 0.28 in CH₂Cl₂); IR (neat): ν=3443, 2250, 1661, 1053, 1005, 821 cm⁻¹; ¹H NMR (600 MHz, CDCl₃): δ 8.64 (s, 1H), 7.55 (s, 1H), 6.28 (d, J=7.6 Hz, 1H), 4.92 (ddd, J=9.8, 7.5, 4.4 Hz, 1H), 4.21 (d, J=4.5 Hz, 1H), 3.83 (d, J=12.6 Hz, 1H), 3.74 (d, J=12.6 Hz, 1H), 3.40 (d, J=9.8 Hz, 1H), 1.53 (s, 3H), 1.49 (s, 3H), 1.42 (s, 3H); ¹³C NMR (150 MHz, CDCl₃): δ 153.2, 151.0, 151.0, 132.6, 118.1, 99.2, 89.9, 79.1, 75.5, 73.9, 66.2, 52.8, 27.4, 23.0, 20.8. HRMS (EI⁺) calcd for C₁₅H₁₈CIlN₃04 [M+H]⁺ 466.0025; found 466.0054

Determination of Relative Stereochemistry for Nucleoside 43

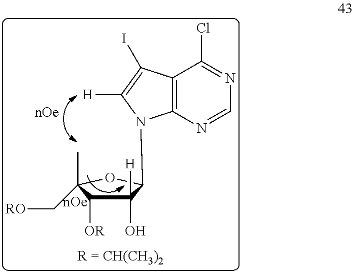

Analysis of 2D NOESY of nucleoside 43 supported the indicated stereochemistry.

Preparation of Nucleoside Analogue 44

Methylmagnesium chloride (3.0 M in THF, 1.56 mL, 4.68 mmol, 2.2 equiv.) was added dropwise to a solution of 41 (syn-/anti-fluorohydrin=3:1, 1.00 g, 2.13 mmol, 1.0 equiv.) at −78° C. in CH₂Cl₂ (20.0 mL). The resulting reaction mixture was stirred at −78° C. for 5 hrs. The reaction mixture was quenched with an ammonium chloride:methanol solution (1:1—saturated ammonium chloride solution:methanol) and warmed to room temperature. The reaction mixture was diluted with CH₂Cl₂ (50 mL) and the organic layer was separated, dried over MgSO₄, filtered, and concentrated under reduced pressure. Purification of crude product 42b by flash chromatography (pentane:ethyl acetate—65:35) afforded 42b (0.498 g, 48%) as an off-white solid.

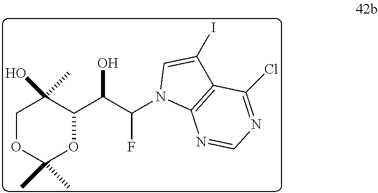

Data for 42b: [α]_{D}²⁰=−17.7 (c 1.8 in CH₂Cl₂); IR (neat): ν=3316, 2991, 1206, 1086, 863, 736 cm⁻¹; ¹H NMR (600 MHz, dmso-d₆): δ 8.76 (s, 1H), 8.28 (s, 1H), 6.92 (dd, J=45.8, 3.3 Hz, 1H), 6.23 (d, J=5.0 Hz, 1H), 4.65 (s, 1H), 4.45 (m, 1H), 3.44 (d, J=11.1 Hz, 1H), 3.28 (d, J=8.0 Hz, 1H), 3.23 (d, J=11.1, 1H), 1.28 (s, 3H), 1.13 (s, 3H), 0.75 (s, 3H); ¹³C NMR (150 MHz, dmso-d₆): δ 151.5, 151.4, 151.2, 134.3, 116.0, 98.3, 90.2 (d, J=202.7 Hz), 74.1 (d, J=4.5 Hz), 70.1 (d, J=25.1 Hz), 70.0, 66.7, 55.2, 28.4, 19.7, 18.1; ¹⁹F NMR (470 MHz, dmso-d₆): δ −151.1. HRMS (EI⁺) calcd for C₁₅H₁₉ClFIN₃O₄[M+H]⁺ 486.0087; found 486.0080

To a stirred solution of 42b (0.100 g, 0.206 mmol, 1.0 equiv.) in dry MeCN (2.0 mL) was added InCl₃ (0.046 g, 0.206 mmol, 1.0 equiv.). The resulting reaction mixture was heated to 50° C. for 2 hrs. 2,2-dimethoxypropane (0.214 mg, 2.06 mmol, 10.0 equiv.) and camphorsulfonic acid (9.6 mg, 0.041 mmol, 0.20 equiv.) were added and the reaction mixture was stirred for a further 1 hr at 50° C. The reaction mixture was then concentrated and purified by flash chromatography (0-10% MeOH in CH₂Cl₂) to afford nucleoside 44 (0.049 g, 51%) as a white solid.

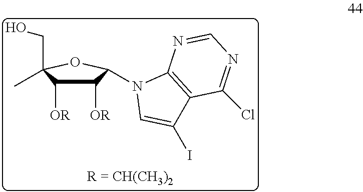

Data for nucleoside analogue 44: [α]_{D}²⁰=+1.4 (c 0.84 in MeOD); ¹H NMR (600 MHz, CDCl₃): δ 8.58 (s, 1H), 7.68 (s, 1H), 6.83 (d, J=4.5 Hz. 1H), 5.01 (dd, J=6.0, 4.7 Hz, 1H), 4.77 (d, J=6.0 Hz, 1H), 3.79 (dd, J=10.9, 5.2 Hz, 1H), 3.74 (dd, J=10.9, 3.6 Hz, 1H), 2.02 (dd, J=5.2, 3.6 Hz, 1H), 1.48 (s, 3H), 1.41 (s, 3H), 1.31 (s, 3H); ¹³C NMR (150 MHz, CDCl₃): δ 152.6, 150.8, 150.3, 134.5, 117.4, 113.2, 85.1, 85.0, 83.0, 81.1, 69.5, 50.8, 25.6, 24.1, 17.4. HRMS (EI⁺) calcd for C₁₅H₁₈CIlN₃O₄[M+H]⁺ 466.0025; found 466.0000

Determination of Relative Stereochemistry for Nucleoside 44

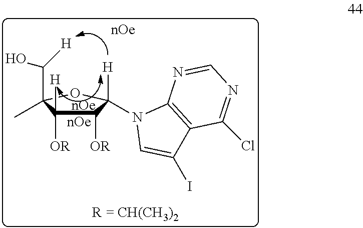

Analysis of 2D NOESY of nucleoside 44 supported the indicated stereochemistry

Preparation of Nucleoside Analogue 45

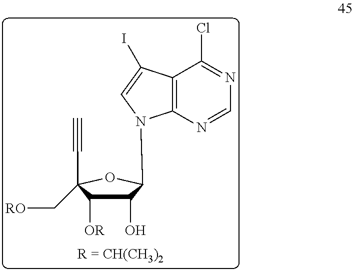

Ethynylmagnesium chloride (0.5 M in THF, 8.94 mL, 4.47 mmol, 2.1 equiv.) was added dropwise to a solution of 41 (syn-/anti-fluorohydrin=3:1, 1.00 g, 2.13 mmol, 1.0 equiv.) at −78° C. in CH₂Cl₂ (10 mL). The reaction mixture was stirred at this temperature for 2 hrs and then allowed to warm gradually to room temperature and stirred for 12 hrs. The reaction mixture was quenched with saturated ammonium chloride solution and diluted with ethyl acetate. The organic layer was separated, dried over MgSO₄, filtered, and concentrated under reduced pressure. Purification of crude product 45 by flash chromatography (0-10% MeOH in CH₂Cl₂) afforded nucleoside 45 (0.415 g, 41%) as a white solid.

Data for nucleoside analogue 45: [α]_{D}²⁰=−29.5 (c 0.58 in MeOH); IR (neat): ν=3291, 2924, 1446, 1201, 1023, 600 cm⁻¹; ¹H NMR (600 MHz, dmso-d₆): δ 8.72 (s, 1H), 8.02 (s, 1H), 6.44 (d, J=8.1 Hz, 1H), 5.05 (dd, J=8.1, 3.6 Hz, 1H), 4.44 (d, J=3.6 Hz, 1H), 4.16 (s, 1H), 4.01 (d, J=13.2 Hz, 1H), 3.82 (d, J=13.2 Hz, 1H), 3.44 (br s, 1H), 1.49 (s, 3H), 1.43 (s, 3H); ¹³C NMR (150 MHz, dmso-d₆): δ 151.7, 151.4, 151.1, 132.8, 116.6, 97.5, 86.5, 81.1, 80.5, 75.0, 74.1, 72.3, 64.2, 53.0, 28.5, 18.9. HRMS (EI⁺) calcd for C₁₆H₁₆CIlN₃04 [M+H]⁺ 475.9869; found 475.9849

Determination of Relative Stereochemistry for Nucleoside 45

The relative stereochemistry was assigned based on comparison of the chemical shift of the anomeric proton with compounds 43 and 46.

Preparation of Nucleoside Analogue 46

Phenylmagnesium chloride (2.0 M in THF, 2.24 mL, 4.47 mmol, 2.1 equiv.) was added dropwise to a solution of 41 (syn-/anti-fluorohydrin=3:1, 1.00 g, 2.13 mmol, 1.0 equiv.) at −78° C. in CH₂Cl₂ (10 mL). The reaction mixture was stirred at this temperature for 2 hrs and then allowed to gradually warm to room temperature and stirred for 12 hrs. The reaction mixture was quenched with saturated ammonium chloride solution and diluted with ethyl acetate. The organic layer was separated, dried over MgSO₄, filtered, and concentrated under reduced pressure. Purification of crude product 46 by flash chromatography (0-10% MeOH in CH₂Cl₂) afforded nucleoside 46 (0.496 g, 45%) as a white solid.

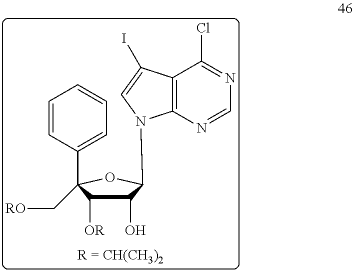

Data for nucleoside analogue 46: [α]_{D}²⁰=−23.6 (c 1.7 in CH₂Cl₂); IR (neat): ν=3309, 2990, 2938, 1575, 1538, 1445, 1200 cm⁻¹; ¹H NMR (600 MHz, dmso-d₆): δ 8.70 (s, 1H), 7.63 (s, 1H), 7.43 (m, 5H), 6.55 (d, J=8.3 Hz, 1H), 5.55 (d, J=6.9 Hz, 1H), 4.77 (d, J=3.8 Hz, 1H), 4.67 (ddd, J=8.3, 6.9, 3.8 Hz, 1H), 3.81 (d, J=12.9 Hz, 1H), 3.68 (d, J=12.9 Hz, 1H), 1.62 (s, 3H), 1.50 (s, 3H); ¹³C NMR (150 MHz, dmso-d₆): δ 152.0, 151.3, 151.0, 140.4, 133.4, 128.5, 128.0, 125.3, 111.8, 97.4, 86.1, 80.8, 73.9, 72.5, 67.0, 54.3, 28.3, 20.2. HRMS (EI⁺) calcd for C₂₀H₂₀CIlN₃O₄[M+H]⁺ 528.0182; found 528.0206.

Determination of Relative Stereochemistry for Nucleoside 46

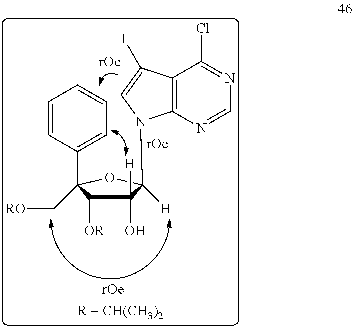

Analysis of 2D NOESY of nucleoside 46 supported the indicated stereochemistry.

Preparation of Nucleoside Analogue 47

Ethynylmagnesium chloride (0.5 M in THF, 8.94 mL, 4.47 mmol, 2.1 equiv.) was added dropwise to a solution of 41 (syn-/anti-fluorohydrin=3:1, 1.00 g, 2.13 mmol, 1.0 equiv.) at −78° C. in CH₂Cl₂ (20 mL). The resulting reaction mixture was stirred at −78° C. for 1 hr. The reaction mixture was quenched with an ammonium chloride:methanol solution (1:1—saturated ammonium chloride solution:methanol) and warmed to room temperature. The reaction mixture was diluted with CH₂Cl₂ (50 mL) and the organic layer was separated, dried over MgSO₄, filtered, and concentrated under reduced pressure. Purification of crude product S47 by flash chromatography (pentane:ethyl acetate—65:35) afforded S47 (0.720 g, 68%, 1:1 mixture of diastereomers) as an off-white solid.

To a stirred solution of S47 (0.050 g, 0.101 mmol, 1.0 equiv.) in dry MeCN (2.0 mL) was added InCl₃ (0.022 g, 0.101 mmol, 1.0 equiv.). The resulting reaction mixture was heated to 50° C. for 2 hrs. 2,2-dimethoxypropane (0.124 mL, 1.01 mmol, 10.0 equiv.) and camphorsulfonic acid (4.7 mg, 0.020 mmol, 0.20 equiv.) were added and the reaction mixture was stirred for a further 1 hr at 50° C. The reaction mixture was then concentrated and purified by flash chromatography (0-10% MeOH in CH₂Cl₂) to afford nucleoside 47 (0.029 g, 60%) as a white solid.

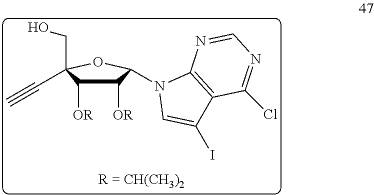

Data for nucleoside analogue 47: [α]_{D}²⁰=+6.3 (c 2.0 in CH₂Cl₂); ¹H NMR (600 MHz, CDCl₃): δ 8.59 (s, 1H), 7.82 (s, 1H), 6.85 (d, J=4.6 Hz, 1H), 5.03 (dd, J=6.0, 4.9 Hz, 1H), 4.98 (d, J=6.0 Hz, 1H), 3.97 (dd, J=11.5, 4.4 Hz, 1H), 3.92 (dd, J=11.5, 3.5 Hz, 1H), 2.82 (s, 1H), 2.18 (dd, J=4.4, 3.5 Hz, 1H), 1.53 (s, 3H), 1.34 (s, 3H); ¹³C NMR (150 MHz, CDCl₃): δ 152.7, 150.9, 1505, 134.6, 117.4, 114.6, 85.3, 83.0, 82.9, 80.6, 78.2, 77.8, 68.7, 51.4, 25.7, 24.5. HRMS (EI⁺) calcd for C₁₆H₁₆CIlN₃O₄[M+H]⁺ 475.9869; found 475.9885

Determination of Relative Stereochemistry for Nucleoside 47

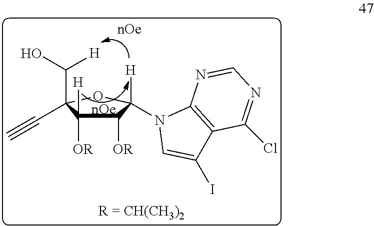

Analysis of 2D NOESY of nucleoside 47 supported the indicated stereochemistry.

Preparation of Nucleoside Analogue 48

Methylmagnesium iodide (3.0 M in THF, 0.39 mL, 1.16 mmol, 3 equiv.) was added dropwise to a solution of A5 (0.100 g, 0.388 mmol, 1 equiv.) at −78° C. in CH₂Cl₂. The resulting reaction mixture was gradually warmed to −10° C. and allowed to stir for 2 hours. Following completion of the reaction as monitored by TLC analysis, the reaction mixture was quenched with saturated ammonium chloride solution and diluted with CH₂Cl₂. The organic layer was subsequently washed twice with water and once with brine. The organic layer was then dried over MgSO₄, filtered, and concentrated under reduced pressure. Purification of crude product S48 by flash chromatography (pentane:ethyl acetate—25:75) afforded S48 (0.089 g, 84%) as a light yellow oil.

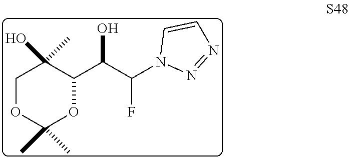

Data for S48: ¹H NMR (600 MHz, CDCl₃): δ 8.16, 8.02, 7.76, 7.76, 6.80, 6.58, 4.62, 4.52, 4.40, 4.31, 4.07, 3.81, 3.59, 3.55, 3.45, 3.25, 3.13, 3.10, 1.52, 1.47, 1.45, 1.40, 1.38, 1.17; ¹³C NMR (150 MHz, CDCl₃): δ 134.2, 134.1, 124.9, 124.3, 99.8, 99.8, 95.6, 93.4, 72.4, 72.4, 71.9, 71.8, 70.2, 70.0, 67.9, 67.8, 28.8, 28.7, 20.0, 19.8, 19.2, 18.5; ¹⁹F NMR (470 MHz, CDCl₃): δ −157.8, −162.8 HRMS (EI⁺) calcd for C₁₁H₁₉FN₃O₄ [M+H]⁺ 276.1354; found 276.1366

To a solution of S48 (0.060 g, 0.218 mmol, 1 equiv.) in dry MeCN (2.18 mL) was added Sc(OTf)₃ (0.268 g, 0.545 mmol, 2.5 equiv.). After stirring the reaction mixture for 16 hrs, 0.50 mL of acetic anhydride and 0.50 mL of pyridine were added to the reaction mixture. The reaction mixture was stirred for a further 4 hrs and then diluted with CH₂Cl₂. The organic layer was washed with twice with 1 M HCl and once with water, dried over sodium sulfate, filtered, and concentrated under reduced pressure to yield crude 48. Purification of crude product 48 by flash chromatography (pentane:ethyl acetate—60:40) afforded 48 (0.024 g, 32% yield).

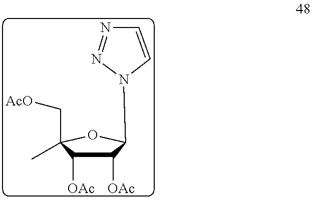

Data for nucleoside analogue 48: [α]_{D}²⁰=+18.4 (c 1.46 in CH₂Cl₂); IR (neat): ν=2925, 1744, 1374, 1215, 1049 cm⁻¹; ¹H NMR (600 MHz, CDCl₃): δ 7.76 (d, J=0.60 Hz, 1H), 7.75 (d, J=0.60 Hz, 1H) 6.19 (d, J=4.7 Hz, 1H), 6.02 (dd, J=5.4, 4.7 Hz, 1H), 5.67 (d, J=5.4 Hz, 1H), 4.17 (d, J=12.0 Hz, 1H), 4.08 (d, J=12.0 Hz, 1H), 2.15 (s, 3H), 2.09 (s, 3H), 2.03 (s, 3H), 1.37 (s, 3H); ¹³C NMR (150 MHz, CDCl₃): δ 170.3, 169.3, 169.2, 134.4, 122.7, 89.4, 85.6, 75.0, 71.9, 67.9, 20.8, 20.5, 20.5, 19.3. HRMS (EI⁺) calcd for C₁₄H₂₀N₃O₇ [M+H]⁺ 342.1296; found 342.1312

Determination of Relative Stereochemistry for Nucleoside Analogue 48

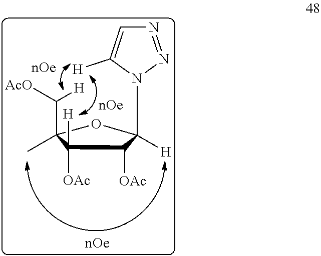

Analysis of 2D NOESY of nucleoside 48 supported the indicated stereochemistry.

Preparation of Nucleoside Analogue 49

Following General Procedure E, p-tolylmagnesium bromide (1.0 M in THF, 0.712 mL, 0.71 mmol) was added to a solution of 59 (0.050 g, 0.158 mmol) in CH₂Cl₂ (6.30 mL) at −78° C. The reaction mixture was stirred for 4.5 hrs. Without further purification, crude S49 was dissolved in MeCN (1.58 mL) and 2 M NaOH (0.198 mL, 0.395 mmol) was added and the reaction mixture was heated to 50° C. for 4 hrs. Purification of crude product 49 by flash chromatography (pentane:ethyl acetate—35:65) afforded nucleoside 49 (0.024 g, 39% yield over two steps) as colorless oil.

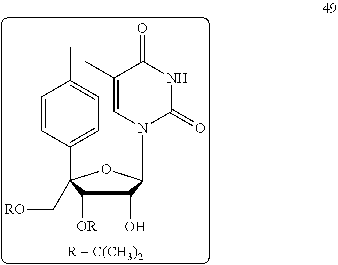

Data for nucleoside analogue 49: [α]_{D}²⁰=−56.5 (c 0.4 in MeOH); IR (neat): ν=3432, 2939, 1700, 1466, 1378, 1129, 1051 cm⁻¹; ¹H NMR (600 MHz, CD₃CN): δ 8.96 (br s, 1H), 7.38 (d, J=8.1 Hz, 2H), 7.26 (d, J=8.1 Hz, 2H), 6.78 (d, J=0.90 Hz, 1H), 6.24 (d, J=8.2 Hz, 1H), 4.76 (d, J=3.8 Hz, 1H), 4.19 (s, 1H), 3.80 (d, J=13.2 Hz, 1H), 3.73 (d, J=13.2 Hz, 1H), 3.48 (br s), 2.35 (s, 3H), 1.68 (d, J=0.90 Hz, 3H), 1.60 (s, 3H), 1.49 (s, 3H); ¹³C NMR (150 MHz, CD₃CN): δ 164.6, 152.8, 139.6, 138.7, 137.4, 130.6, 126.7, 112.0, 99.2, 88.9, 81.6, 74.9, 74.3, 68.7, 29.0, 21.4, 20.8, 12.8. HRMS (EI⁺) calcd for C₂₀H₂₅N₂O₆ [M+H]⁺ 389.1707; found 389.1707

Determination of Relative Stereochemistry for Nucleoside 49

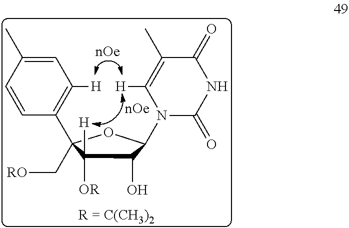

Analysis of 2D NOESY of nucleoside 49 supported the indicated stereochemistry

Preparation of Nucleoside Analogue 50

Following General Procedure E, cyclopropylmagnesium bromide (1.0 M in 2-methylTHF, 0.79 mL, 0.79 mmol, 5 equiv.) was added to a solution of 59 (0.050 g, 0.158 mmol, 1 equiv.) in CH₂Cl₂ (6.30 mL) at −78° C. The reaction mixture was stirred for 5 hrs. Without further purification, crude S50 was dissolved in MeCN (1.60 mL) and 2 M NaOH (0.193 mL, 0.395 mmol) was added and the reaction mixture was stirred for 4 hrs at 50° C. Purification of crude product 50 by flash chromatography (pentane:ethyl acetate—30:70) afforded nucleoside 50 (0.021 g, 40% yield) as an off-white solid.

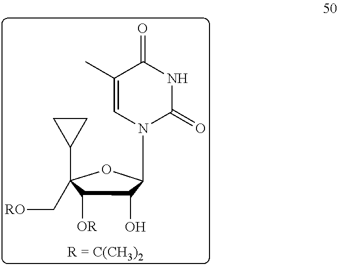

Data for nucleoside analogue 50: [α]_{D}²⁰=−32.6 (c 0.47 in CH₂Cl₂); IR (neat): ν=3500, 3251 2997, 2175, 1690, 1088, 888 cm⁻¹; ¹H NMR (600 MHz, CDCl₃): δ 7.10 (s, 1H), 6.04 (d, J=7.9 Hz, 1H), 4.25 (dd, J=7.9, 5.1 Hz. 1H), 4.08 (d, J=5.1 Hz, 1H), 3.70 (d, J=11.9 Hz, 1H), 3.63 (d, J=11.9 Hz, 1H), 3.15 (br s, 1H), 1.93 (s, 3H), 1.44 (s, 3H), 1.43 (s, 3H), 1.21 (m, 1H), 0.63 (m, 1H), 0.55 (m, 1H), 0.46 (m, 1H), 0.42 (m, 1H); ¹³C NMR (150 MHz, CDCl₃): δ 163.3, 151.0, 134.9, 111.9, 100.1, 87.5, 81.2, 74.0, 72.5, 64.3, 25.9, 25.6, 16.2, 12.9, 1.31, 0.50. HRMS (EI⁺) calcd for C₁₆H₂₂N₂O₆ [M+H]⁺ 339.1551; found 339.1575

Determination of Relative Stereochemistry for Nucleoside 50

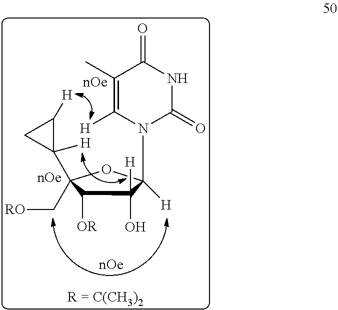

Analysis of 2D NOESY of nucleoside 50 supported the indicated stereochemistry.

Preparation of Nucleoside Analogue 51

Following General Procedure E, p-methoxyphenylmagnesium bromide (0.5 M in THF, 1.58 mL, 0.79 mmol, 5 equiv.) was added to a solution of 59 (0.050 g, 0.158 mmol, 1 equiv.) in CH₂Cl₂ (6.30 mL) at −78° C. The reaction mixture was stirred for 5 hrs. Without further purification, crude S51 was dissolved in MeCN (1.60 mL) and 2 M NaOH (0.193 mL, 0.395 mmol) was added and the reaction mixture was stirred for 4 hrs at 50° C. Purification of crude product 51 by flash chromatography (pentane:ethyl acetate—30:70) afforded nucleoside 51 (0.026 g, 41% yield) as a white solid.

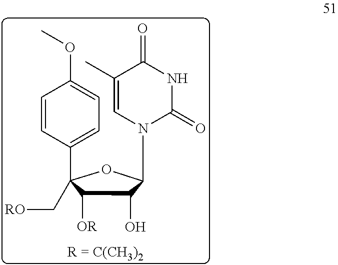

Data for nucleoside analogue 51: [α]_{D}²⁰=−52.8 (c 1.0 in CH₂Cl₂); IR (neat): ν=3197, 2990, 1693, 1252, 1036, 834 cm⁻¹; ¹H NMR (600 MHz, CDCl₃): δ 7.38 (d, J=8.7 Hz, 2H), 6.96 (d, J=8.7 Hz, 2H), 6.78 (s, 1H), 6.37 (d, J=7.9 Hz, 1H), 4.75 (d, J=4.1 Hz, 1H), 4.16 (m, 1H), 3.87 (d, J=13.1 Hz, 1H), 3.84 (s, 3H), 3.79 (d, J=13.1, 1H), 2.99 (br s, 1H), 1.63 (s, 3H), 1.56 (s, 3H); ¹³C NMR (150 MHz, CDCl₃): δ 163.2, 159.9, 151.1, 135.8, 131.8, 126.5, 114.5, 111.7, 98.7, 88.7, 80.6, 74.8, 73.2, 67.7, 55.6, 28.1, 20.4, 12.7. HRMS (EI⁺) calcd for C₂₀H₂₅N₂O₇ [M+H]⁺ 405.1656; found 405.1650

Determination of Relative Stereochemistry for Nucleoside Analogue 51

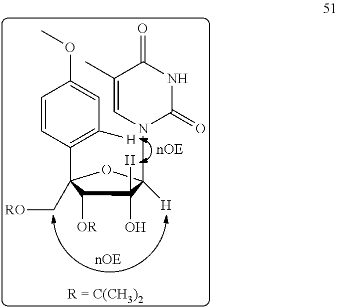

Analysis of 2D NOESY of nucleoside 51 supported the indicated stereochemistry.

Preparation of Nucleoside Analogue 52

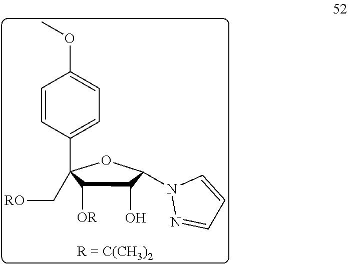

Following General Procedure E, p-methoxyphenylmagnesium bromide (0.5 M in THF, 4.66 mL, 2.33 mmol, 3 equiv.) was added to a solution of A1 (0.200 g, 0.775 mmol, 1 equiv.) in CH₂Cl₂ (7.75 mL) at −78° C. The reaction mixture was stirred for 6 hrs. Crude S52 was purified by flash chromatography (ethyl acetate-pentane—4:6) to yield S52 (0.157 g, 55% yield). S52 (0.155 g, 0.423 mmol, 1 equiv.) was dissolved in MeCN (2.82 mL) and 2 M NaOH (0.53 mL, 1.06 mmol, 2.5 equiv.) was added and the reaction mixture was stirred for 5 hrs at 50° C. Purification of crude nucleoside analogue 52 by flash chromatography (pentane:ethyl acetate—40:60) afforded 52 (0.085 g, 58% yield) as a light orange oil. Data for nucleoside analogue 52: [α]_{D}²⁰=−14.8 (c 1.4 in CH₂Cl₂); IR (neat): ν=3418, 2991, 1611, 1512, 1250, 1032, 759 cm⁻¹; ¹H NMR (600 MHz, CD₃CN): δ 7.69 (d, J=2.7 Hz, 1H), 7.56 (d, J=1.4 Hz, 1H), 7.39 (d, J=8.9 Hz, 2H), 6.91 (d, J=8.9 Hz, 2H), 6.35 (dd, J=2.7, 1.4 Hz, 1H), 5.99 (d, J=7.9 Hz, 1H), 4.73 (dd, J=7.9, 3.7 Hz, 1H), 4.59 (d, J=3.7 Hz, 1H), 3.92 (d, J=13.3 Hz, 1H), 3.78 (s, 3H), 3.68 (d, J=13.3 Hz, 1H), 1.62 (s, 3H), 1.51 (s, 3H); ¹³C NMR (150 MHz, CD₃CN): δ 160.6, 141.5, 133.7, 132.1, 128.4, 114.8, 107.6, 99.1, 93.9, 82.0, 75.9, 75.1, 68.9, 56.3, 29.0, 21.2. HRMS (EI⁺) calcd for C₁₈H₂₃N₂O₅ [M+H]⁺ 347.1601; found 347.1610

Determination of Relative Stereochemistry for Nucleoside 52

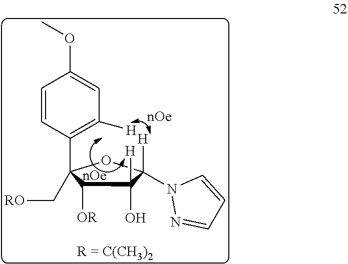

Analysis of 2D NOESY of nucleoside 52 supported the indicated stereochemistry.

Preparation of Nucleoside Analogue 53

Following General Procedure E, methylmagnesium bromide (3.0 M in THF, 0.258 mL, 0.78 mmol, 4 equiv.) was added to a solution of A1 (0.050 g, 0.194 mmol, 1 equiv.) in CH₂Cl₂ (3.90 mL) at −78° C. The reaction mixture was stirred for 6 hrs. Crude S53 was purified by flash chromatography (ethyl acetate-pentane—6:4) to yield S53 (0.026 g, 49% yield). S53 (0.030 g, 0.109 mmol) was dissolved in MeCN (1.09 mL) and 2 M NaOH (0.545 mL, 1.09 mmol, 10 equiv.) was added and the reaction mixture was stirred for 5 hrs at 50° C. Purification of crude nucleoside analogue 53 by flash chromatography (pentane:ethyl acetate—25:75) afforded 53 (0.017 g, 61% yield) as a light yellow oil.

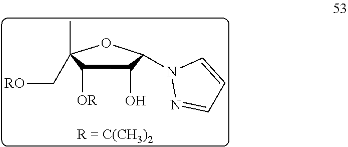

Data for nucleoside analogue 53: [α]_{D}²⁰=+11.3 (c 0.38 in CH₂Cl₂)); IR (neat): ν=3383, 2992, 2922, 1382, 1199, 1090, 908 cm^{-1 1}H NMR (600 MHz, CDCl₃): δ 7.60 (d, J=2.4 Hz, 1H), 7.59 (d, J=1.6 Hz, 1H), 6.35 (dd, J=2.4, 1.6 Hz, 1H), 5.29 (d, J=1.3 Hz, 1H), 4.12 (dd, J=3.0, 1.3 Hz, 1H), 3.98 (d, J=3.0 Hz, 1H), 3.76 (d, J=11.3 Hz, 1H), 3.52 (d, J=11.3 Hz, 1H), 1.47 (s, 3H), 1.44 (s, 3H), 1.41 (s, 3H); ¹³C NMR (150 MHz, CDCl₃): δ 141.3, 129.3, 107.4, 99.6, 72.6, 70.3, 67.3, 64.9, 57.0, 28.8, 20.5, 19.0. HRMS (EI⁺) calcd for C₁₂H₁₉N₂O₄ [M+H]⁺ 255.1339; found 255.1320

Determination of Relative Stereochemistry for Nucleoside Analogue 53

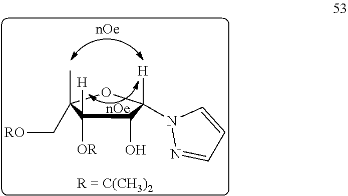

Analysis of 2D NOESY of nucleoside 53 supported the indicated stereochemistry

Preparation of Nucleoside Analogue 54

p-Chlorophenylmagnesium bromide (1.0 M in diethyl ether, 4.32 mL, 4.32 mmol, 3.2 equiv.) was added dropwise to a stirred solution of fluorohydrin aldol adduct A6 (0.500 g, 1.35 mmol, 1 equiv.) in THF (10.0 mL) at 0° C. The resulting reaction mixture was stirred for 14 hrs at room temperature and for a further 8 hrs at 40° C. The reaction mixture was then diluted with ethyl acetate (100 mL) and washed once with water (100 mL) and once with brine (50 mL). The organic layer was separated, dried over MgSO₄, filtered, and concentrated under reduced pressure to give crude 54. Purification of crude nucleoside analogue 54 by flash chromatography (pentane:ethyl acetate—50:50) afforded 54 (0.289 g, 46%).

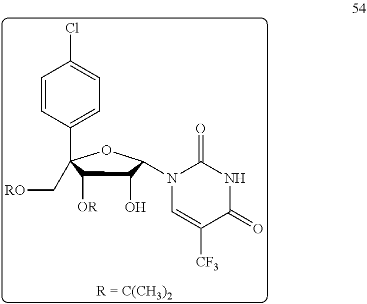

Data for nucleoside 54: [α]_{D}²⁰=+10.5 (c 0.8 in CH₂Cl₂); IR (neat): ν=3087, 2996, 1699, 1467, 1283, 1129, 1085 cm⁻¹; ¹H NMR (600 MHz, dmso-d₆): δ 11.94 (br s, 1H), 8.74 (s, 1H), 7.57 (d, J=8.7 Hz, 2H), 7.50 (d, J=8.7 Hz, 2H), 6.13 (d, J=7.2 Hz, 1H), 5.67 (br s, 1H), 4.66 (d, J=4.3 Hz, 1H), 4.17 (dd, J=6.8, 4.3 Hz, 1H), 3.98 (d, J=13.4 Hz, 1H), 3.88 (d, J=13.4 Hz, 1H), 1.63 (s, 3H), 1.40 9 s, 3H); ¹³C NMR (150 MHz, CD₃CN): δ 159.9, 150.5, 144.3 (q, J=5.9 Hz), 138.0, 134.9, 129.9, 128.1, 124.1 (q, J=269.0 Hz), 104.0 (q, J=32.0), 99.6, 84.7, 81.6, 73.6, 73.6, 67.7, 28.6, 19.9; ¹⁹F NMR (470 MHz, CD₃CN): δ −62.9. HRMS (EI⁺) calcd for C₁₉H₁₉ClF₃N₂O₆ [M+H]⁺ 463.0878; found 463.0875

Determination of Relative Stereochemistry for Nucleoside 54

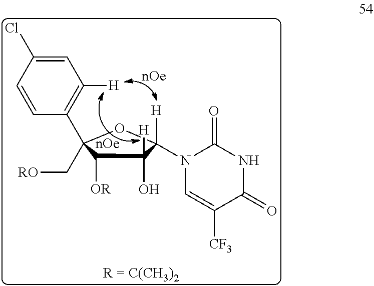

Analysis of 2D NOESY of nucleoside 54 supported the indicated stereochemistry.

Preparation of Nucleoside Analogue 57

To a solution of nucleoside 35 (0.285 g, 1.0 mmol, 1.0 equiv.) in dry dioxane (20 mL) was added (diacetoxyiodo)benzene (0.805 g, 2.5 mmol, 2.5 equiv.) and TEMPO (0.031 g, 0.20 mmol, 0.2 equiv.). The reaction mixture was stirred for 24 hrs at room temperature until complete consumption of starting material was detected by TLC analysis. The reaction mixture was concentrated to 2 mL and purified with flash chromatography (CH₂Cl₂:Et₂O—75:25) to afford ketone 56 (0.265 g, 0.94 mmol, 94% yield) as a white solid. Ketone 56 (0.053 g, 0.19 mmol, 1.0 equiv.) was dissolved in methanol (0.94 mL) and 3 drops of AcCl were added. The solution was stirred for 12 hrs at room temperature until complete consumption of starting material was detected by TLC analysis. The reaction mixture was concentrated under reduced pressure to a white solid S57. The spectral data matched previous reports (50). The crude product was subsequently dissolved in tetrahydrofuran (4.0 mL) and the resulting solution was cooled to −78° C. and methyl magnesium bromide (3.0 M in THF, 0.38 mL, 1.13 mmol, 6.0 equiv.) was added. The resulting brown suspension was stirred at −78° C. for 3 hrs. The reaction mixture was quenched at −78° C. with a solution of methanol:TFA (10:1) and then concentrated under reduced pressure. The crude product 57 was purified by flash chromatography (CH₂Cl₂:MeOH—85:15) to yield nucleoside analogue (0.024 g, 49% yield) as a white solid. The spectral data matched previous reports (51).

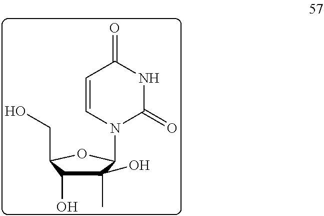

Data for nucleoside analogue 57: ¹H NMR (600 MHz, MeOD): δ 7.86 (d, J=8.1 Hz, 1H), 5.96 (s, 1H), 5.64 (d, J=8.1 Hz, 1H), 3.85 (m, 4H), 1.29 (s, 3H). HRMS (EI⁺) calcd for C₁₀H₁₅N₂O₆ [M+H]⁺ 259.0925; found 259.0915

Preparation of Nucleoside Analogue 60

To a stirred solution of 59 (0.100 g, 0.316 mmol, 1 equiv.) in THF (3.10 mL) was added BnNH₂ (0.086 ml, 0.790 mmol, 2.5 equiv) and glacial acetic acid (18.2 μl, 0.316 mmol, 1 equiv.), and the resulting mixture was stirred at 20° C. for 1 hr. NaBH₃CN (0.050 g, 0.79 mmol, 2.5 equiv.) was then added and the mixture was stirred for an additional hr. The reaction mixture was then diluted with CH₂Cl₂ to a concentration of 0.05M and treated with water. The layers were separated, and the organic layer was washed with brine, dried with MgSO₄, and concentrated under reduced pressure. The resulting product S60 was used without any further purification. To a stirred solution of S60 in MeCN (8.7 mL) was added 2 M NaOH (0.240 mL, 0.478 mmol, 1.1 equiv.). The reaction mixture was stirred for 14 hrs at room temperature. The reaction mixture was then diluted with CH₂Cl₂ and quenched with saturated ammonium chloride solution. The organic layer was washed with saturated ammonium chloride solution and water, dried over MgSO₄, filtered, and concentrated under reduced pressure. Crude 60 was purified by flash chromatography (ethyl acetate:pentane −80:20) to afford nucleoside analogue 60 (0.060 g, 49% yield over two steps) as a light yellow oil.

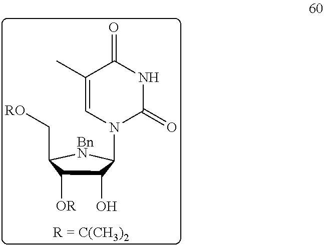

Data for nucleoside analogue 60: [α]_{D}²⁰=−15.5 (c 0.53 in CH₂Cl₂); IR (neat): ν=2990, 1670, 1382, 1200, 1078, 701 cm⁻¹; ¹H NMR (600 MHz, CDCl₃): δ 7.23-7.32 (m, 4H), 7.19 (d, J=7.0 Hz, 2H), 5.07 (s, 1H), 4.11 (d, J=4.8 Hz, 1H), 3.81 (d, J=12.9 Hz, 1H), 3.77 (d, J=12.9 Hz, 1H), 3.72 (dd, J=10.4, 4.6 Hz, 1H), 3.67 (dd, J=10.4, 10.2 Hz, 1H), 3.61 (dd, J=9.8, 4.8 Hz, 1H), 3.11 (ddd, J=10.2, 9.8, 4.6 Hz, 1H), 1.86 (s, 3H), 1.49 (s, 3H), 1.46 (s, 3H); ¹³C NMR (150 MHz, CDCl₃): δ 163.5, 150.7, 136.8, 135.9, 129.1, 128.7, 128.2, 110.1, 101.0, 83.1, 74.9, 73.2, 66.6, 58.1, 58.0, 29.2, 19.9, 12.8. HRMS (EI⁺) calcd for C₂₀H₂₆N₃O₅ [M+H]⁺ 388.1867; found 388.1843.

Determination of Relative Stereochemistry for Nucleoside 60

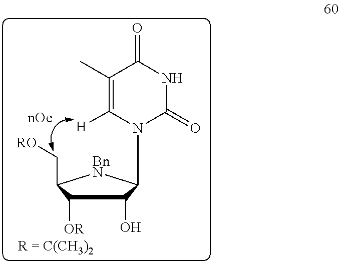

Analysis of 2D NOESY of nucleoside 60 supported the indicated stereochemistry

Preparation of Nucleoside Analogue 61

Following General Procedure E, allylmagnesium bromide (1.0 M in diethyl ether, 1.42 mL, 1.42 mmol, 4.5 equiv.) was added to a solution of 59 (0.100 g, 0.316 mmol, 1 equiv.) in CH₂Cl₂ (12.6 mL) at −78° C. The reaction mixture was stirred for 5 hrs. Without further purification, crude S61 was dissolved in MeCN (3.16 mL) and 2 M NaOH (0.395 mL, 0.79 mmol, 2.5 equiv.) was added and the reaction mixture was stirred for 4 hrs at 50° C. Purification of crude 61 by flash chromatography (CH₂Cl₂:MeOH—4:96) afforded nucleoside analogue 61 (0.050 g, 47% yield) as a dark orange oil.

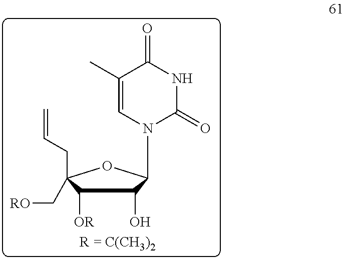

Data for nucleoside analogue 61: [α]_{D}²⁰=−6.0 (c 0.4 in MeOH); IR (neat): ν=3340, 2992, 1670, 1376, 1044 cm⁻¹; ¹H NMR (600 MHz, CD₃CN): δ 8.95 (br s, 1H), 7.27 (s, 1H), 6.02 (d, J=8.3 Hz, 1H), 5.87 (m, 1H), 5.22 (d, J=17.7 Hz, 1H), 5.20 (d, J=10.1 Hz, 1H), 4.31 (ddd, J=9.3, 8.3, 4.9 Hz, 1H), 4.11 (d, J=4.9 Hz, 1H), 3.68 (d, J=12.2 Hz, 1H), 3.64 (d, J=12.2 Hz, 1H), 3.41 (d, J=9.3 Hz, 1H), 2.50 (dd, J=14.2, 6.7 Hz, 1H), 2.41 (dd, J=14.2, 8.1 Hz, 1H), 1.85 (s, 3H), 1.40 (s, 3H), 1.39 (s, 3H); ¹³C NMR (150 MHz, CD₃CN): δ 164.6, 152.3, 136.8, 133.7, 120.4, 112.3, 100.3, 88.4, 81.7, 73.9, 73.3, 65.3, 41.7, 26.9, 22.4, 12.8. HRMS (EI⁺) calcd for C₁₆H₂₂N₂O₆ [M+H]⁺ 339.1551; found 339.1556

Determination of Relative Stereochemistry for Nucleoside 61

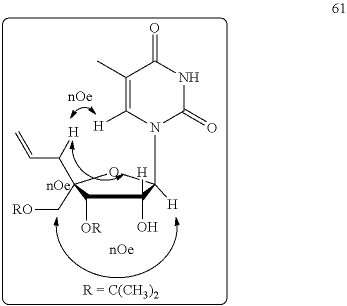

Analysis of 2D NOESY of nucleoside 61 supported the indicated stereochemistry

Preparation of Nucleoside Analogue 62

To a solution of nucleoside 61 (0.022 g, 0.061 mmol, 1 equiv.) in dry THF (0.61 mL) was added 1, 1′-thiocarbonyldiimidazole (0.022 g, 0.122 mmol, 2 equiv.). The reaction mixture was stirred for 18 hrs. Subsequently, CH₂Cl₂ (5 mL) was added to the reaction mixture and washed with water 3 times. The organic layer was dried over MgSO₄, filtered, and concentrated under reduced pressure to yield crude S62. Purification of crude S62 by flash chromatography (pentane:ethyl acetate—40:60) afforded S62 (0.018 g, 66% yield). To a solution of nucleoside S62 (0.014 g, 0.031 mmol, 1 equiv.) in dry toluene (4.45 mL) under nitrogen was added tributyltin hydride (8.35 μL, 0.031 mmol, 1 equiv.) and AIBN (5.1 mgs, 0.031 mmol, 1.0 equiv.). The resulting reaction mixture was purged with nitrogen for 30 minutes. Subsequently, the reaction mixture was stirred for 16 hrs at 90° C. Upon competition, CH₂Cl₂ was added to reaction mixture and the washed with water. The organic layer was dried over MgSO₄, filtered, and concentrated under reduced pressure to yield crude 62. Purification of crude 62 by flash chromatography (ethyl acetate) afforded nucleoside analogue 62 (6.0 mg, 61%) as a white solid.

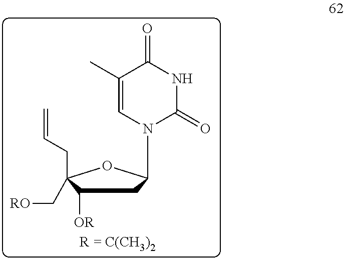

Data for nucleoside analogue 62: [α]_{D}²⁰=+13.3 (c 0.46 in CH₂Cl₂); IR (neat): ν=2924, 1690, 1467, 1375, 1263, 1226, 1053 cm⁻¹; ¹H NMR (600 MHz, CDCl₃): δ 8.26 (s, 1H), 7.31 (d, J=1.1 Hz, 1H), 6.38 (dd, J=9.6, 4.8 Hz, 1H), 5.86 (m, 1H), 5.25-5.27 (m, 2H), 4.22 (d, J=5.2 Hz, 1H), 3.69 (d, J=12.0 Hz, 1H), 3.64 (d, J=12.0 Hz, 1H), 2.50 (m, 2H), 2.41 (dd, J=13.5, 4.8 Hz, 1H), 2.00 (dd, J=13.5, 9.6, 5.2 Hz, 1H), 1.92 (s, 3H), 1.37 (s, 6H); ¹³C NMR (150 MHz, CDCl₃): δ 163.3, 150.0, 135.0, 131.9, 120.2, 111.1, 99.5, 85.8, 84.0, 73.9, 63.9, 40.9, 37.8, 25.6, 22.5, 12.7 HRMS (EI⁺) calcd for C₁₆H₂₃N₂O₅ [M+H]⁺ 323.1601; found 323.1580

Preparation of Fluorohydrins 63 and 64

Following General Procedure E, ethynylmagnesium chloride (0.5 M in THF, 3.5 mL, 1.75 mmol, 3.5 equiv.) was added to a solution of 59 (0.160 g, 0.50 mmol, 1 equiv.) in CH₂Cl₂ (25.0 mL) at −78° C. The reaction mixture was stirred for 4 hrs. The crude products 63 and 64 were purified by flash chromatography (ethyl acetate:hexane—70:30) to afford 63 (0.072 g, 42% yield) and 64 (0.058 g, 34% yield) as white solids.

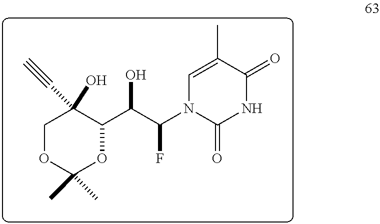

Data for fluorohydrin 63: [α]_{D}²⁰=−60.8 (c 0.4 in MeOH); IR (neat): ν=3320, 2944, 2832, 1670, 1449, 1022, 638 cm⁻¹; ¹H NMR (600 MHz, dmso-d₆): δ 11.47 (br s, 1H), 7.56 (s, 1H), 6.36 (dd, J=43.7, 4.1 Hz, 1H), 6.21 (d, J=5.3 Hz, 1H), 5.37 (br s, 1H), 4.14 (m, 1H), 3.71 (d, J=8.7 Hz, 1H), 3.68 (br s, 1H), 3.42 (s, 1H), 3.16 (d, J=5.0 Hz, 1H), 1.78 (s, 3H), 1.33 (s, 3H), 1.21 (s, 3H); ¹³C NMR (150 MHz, dmso-d₆): δ 163.6, 150.0, 136.7, 109.2, 98.8, 92.7 (d, J=206.6 Hz), 83.7, 76.2, 72.8 (d, J=2.8 Hz), 71.2 (d, J=24.6 Hz), 68.2, 65.7, 27.8, 18.7, 12.1; ¹⁹F NMR (470 MHz, dmso-d₆): δ −170.5 HRMS (EI⁺) calcd for C₁₅H₂₀N₂O₆ [M+H]⁺ 343.1300; found 343.1298.

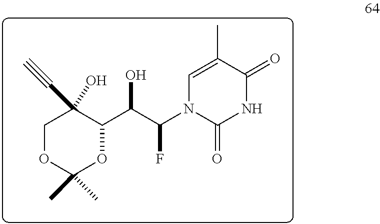

Data for fluorohydrin 64: [α]_{D}²⁰=−38.0 (c 1.2 in MeOH); IR (neat): ν=IR (neat): ν=3395, 2994, 1694, 1468, 1381, 1282, 1043 cm⁻¹; ¹H NMR (600 MHz, CD₃CN): δ 9.29 (br s, 1H), 7.41 (s, 1H), 6.40 (dd, J=43.4, 4.6 Hz, 1H), 4.54 (m, 1H), 4.27 (m, 1H), 4.22 (m, 1H), 3.82 (d, J=9.5 Hz, 1H), 3.79 (d, J=11.5 Hz, 1H), 3.75 (d, J=11.5 Hz, 1H), 2.81 (s, 1H), 1.85 (s, 3H), 1.41 (s, 3H), 1.28 (s, 3H); ¹³C NMR (150 MHz, CD₃CN): δ 164.8, 151.4, 137.7, 111.5, 100.8, 94.1 (d, J=206.9 Hz), 84.4, 75.7, 73.6 (d, J=3.8 Hz), 73.4 (d, J=24.7 Hz), 69.3, 67.3, 28.8, 19.4, 12.8; ¹⁹F NMR (470 MHz, CD₃CN): δ −175.5 HRMS (EI⁺) calcd for C₁₅H₂₀N₂O₆ [M+H]⁺ 343.1300; found 343.1305

Preparation of Nucleoside Analogue 65

Following General Procedure C, a solution of 63 (0.100 g, 0.292 mmol, 1.0 equiv.) and NaOH (29.2 mg, 0.73 mmol, 2.5 equiv.) in MeCN (2.0 mL) was heated to 50° C. for 36 hrs. Purification of the crude 65 by flash chromatography (0-10% MeOH in dichloromethane) afforded nucleoside analogue 65 (58.6 mg, 62% yield) as a white powder.

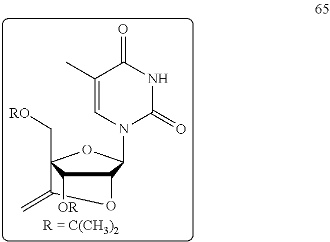

Data for nucleoside analogue 65: [α]_{D}²⁰=−8.7 (c 0.6 in CH₂Cl₂); IR (neat): ν=2994, 1748, 1690, 1270, 1043 cm⁻¹; ¹H NMR (600 MHz, dmso-d₆): δ 11.42 (s, 1H), 7.61 (d, J=1.3 Hz, 1H), 5.46 (s, 1H), 4.86 (s, 1H), 4.63 (d, J=11.2 Hz, 1H), 4.45 (d, J=2.6 Hz, 1H), 4.37 (d, J=2.6 Hz, 1H), 4.23 (d, J=11.2 Hz, 1H), 3.91 (s, 1H), 1.84 (s, 3H), 1.51 (s, 3H), 1.30 (s, 3H); ¹³C NMR (150 MHz, dmso-d₆): δ 163.8, 158.8, 150.0, 135.0, 109.3, 100.4, 87.2, 83.0, 78.4, 76.5, 71.9, 58.5, 28.7, 19.5, 12.0 HRMS (EI⁺) calcd for C₁₅H₁₉N₂O₆ [M+H]⁺ 323.1238; found 323.1235

Preparation of Nucleoside Analogue 68

Following General Procedure C, a solution 64 (0.220 g, 0.64 mmol, 1 equiv.) and 2M NaOH (0.640 mL, 1.28 mmol, 2.0 equiv.) was heated to 50° C. and stirred for 24 hours in MeCN (6.4 mL). Purification of the crude 66 by flash chromatography (MeOH:CH₂Cl₂ —3:97) afforded nucleoside analogue 66 (0.144 mg, 70% yield) as a white powder.

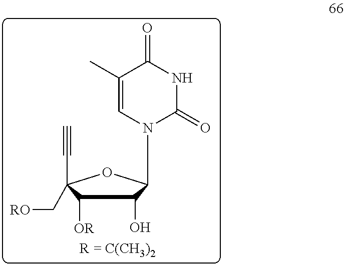

Data for nucleoside analogue 66: [α]_{D}²⁰=+30.8 (c 1.66 in CH₂C₁₂); ¹H NMR (600 MHz, CD₃CN): δ 9.06 (br s, 1H), 7.48 (s, 1H), 6.16 (d, J=8.2 Hz, 1H), 4.61 (ddd, J=8.4, 8.2, 3.7 Hz, 1H), 4.41 (d, J=3.7 Hz, 1H), 4.06 (d, J=13.3 Hz, 1H), 3.88 (d, J=13.3 Hz, 1H), 3.64 (d, J=8.4 Hz, 1H), 3.29 (s, 1H) 1.86 (s, 3H), 1.48 (s, 3H), 1.43 (s, 3H); ¹³C NMR (150 MHz, CD₃CN): δ 164.7, 152.5, 136.9, 112.7, 99.3, 89.4, 81.2, 80.8, 76.5, 75.5, 73.9, 65.9, 29.1, 19.7, 13.1. HRMS (EI⁺) calcd for C₁₅H₁₉N₂O₆ [M+H]⁺ 323.1238; found 323.1245

Determination of Relative Stereochemistry for Nucleoside 66

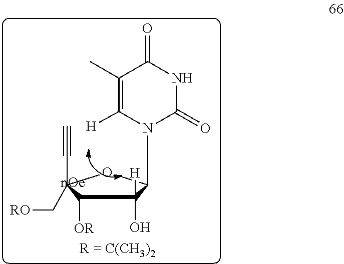

Analysis of 2D NOESY of nucleoside 66 supported the indicated stereochemistry

A solution of 66 (0.050 g, 0.155 mmol, 1 equiv.) in dry CH₂Cl₂ (0.78 mL) was cooled to 0° C. and diethylaminosulfur trifluoride (0.102 mL, 0.776 mmol, 5 equiv.) was added dropwise over 5 minutes. The resulting reaction mixture was slowly warmed to room temperature over 3 hrs. Following completion of the reaction, as monitored by TLC analysis, the reaction mixture was diluted with 5 mL of ethyl acetate and washed with 3 mL of H₂O (3×). Subsequently, the organic layer was dried over MgSO₄, filtered, and concentrated under reduced pressure. Purification of the crude product by flash chromatography (ethyl acetate) afforded nucleoside analogue S66 (0.043 g, 91%) as a white solid.

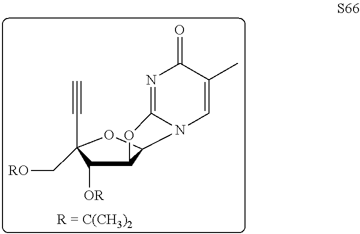

Data for nucleoside analogue S66: [α]_{D}²⁰=−47.5 (c 1.1 in MeCN); IR (neat): ν=3284, 3002, 1626, 1554, 1497, 1134, 1066, 1030 cm⁻¹; ¹H NMR (600 MHz, CD₃CN): δ 7.46 (s, 1H), 6.32 (d, J=5.3 Hz, 1H), 5.13 (d, J=5.3 Hz, 1H), 4.74 (s, 1H), 4.10 (d, J=13.7 Hz, 1H), 4.00 (d, J=13.7 Hz, 1H), 2.87 (s, 1H), 1.87 (s, 3H), 1.47 (s, 3H), 1.34 (s, 3H); ¹³C NMR (150 MHz, CD₃CN): δ 173.1, 161.5, 132.3, 119.6, 99.7, 91.8, 87.4, 79.8, 79.1, 77.8, 74.6, 64.9, 29.0, 19.3, 14.4. HRMS (EI⁺) calcd for C₁₅H₁₇N₂O₅ [M+H]⁺ 305.1132; found 305.1108

To a solution of S66 (0.042 g, 0.138 mmol, 1 equiv.) in wet MeCN (2.76 mL) was added InCl₃ (0.122 g, 0.553 mmol, 4 equiv.). The resulting reaction mixture was heated to 50° C. and was stirred for 16 hrs or until the reaction was complete as monitored by TLC. The reaction mixture was concentrated under reduced pressure and purified by flash chromatography (MeOH:CH₂Cl₂— 7.5:92.5) to afford S68 (0.038 g, 96%). To a solution of S68 (0.038 g, 0.133 mmol, 1 equiv.) in DMF (1.73 mL) was added K₂CO₃ (0.096 g, 0.69 mmol, 5 equiv.). The resulting reaction mixture was heated to 90° C. and stirred for 7 days or until the reaction was complete as monitored by ¹H NMR spectroscopy. Subsequently, the reaction mixture was filtered, concentrated under reduced pressure, and the crude product was purified by flash column chromatography (MeOH:CH₂Cl₂— 10:90) to afford 68 (0.027 g, 71%) as a white solid.

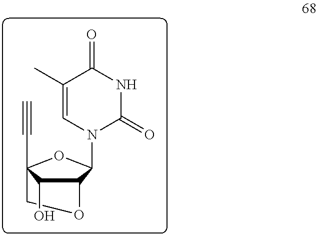 [SMILES: C#C[C@]12CO[C@@H](C1O)[C@H](n1cc(C)c(=O)[nH]c1=O)O2]

Data for nucleoside analogue 68: [α]_{D}²⁰=+16.9 (c 1.0 in MeOH); IR (neat): ν=3261, 2988, 1686, 1272, 1203, 1047, 799 cm⁻¹; ¹H NMR (600 MHz, CD₃CN): δ 9.43 (br s, 1H), 7.31 (d, J=1.1 Hz, 1H), 5.48 (s, 1H), 4.27 (s, 1H), 4.15 (s, 1H), 4.03 (d, J=8.0 Hz, 1H), 3.93 (d, J=8.0 Hz, 1H), 3.16 (s, 1H), 1.85 (d, J=1.1 Hz, 3H); ¹³C NMR (150 MHz, CD₃CN): δ 165.1, 151.4, 135.6, 111.0, 88.6, 80.9, 80.3, 80.2, 75.8, 75.2, 75.1, 13.0. HRMS (EI⁺) calcd for C₁₂H₁₃N₂O₅ [M+H]⁺ 265.0819; found 265.0813

General Procedure F (α-Fluorination/Aldol Reaction with Cyclohexanone/Thiopyranone 35)

A sample of aldehyde (1.0 equiv.) was added to a stirred suspension of NFSI (1.0 equiv.), L-proline (1.0 equiv.), and NaHCO₃ (1.0 equiv.) in DMF (0.75 M) at −10° C. When complete conversion to the α-fluoroaldehyde was observed by ¹H NMR spectroscopic analysis, cyclohexanone or thiopyranone 35 (5.0-10.0 equiv.) was then added and the resulting mixture was warmed gradually to room temperature. After a total of 18 hrs, the reaction mixture was diluted with Et₂O and the organic layer was washed twice with water and once with brine. The organic layer was then dried over MgSO₄, concentrated under reduced pressure and the crude product was purified by flash chromatography as indicated.

Preparation of Syn-Fluorohydrin 68a and Anti-Fluorohydrin 68b

Following General Procedure F, a solution of aldehyde (2.00 g, 5.86 mmol, 1.0 equiv.), NFSI (1.85 g, 5.86 mmol, 1.0 equiv.), L-proline (0.674 g, 5.86 mmol, 1.0 equiv.) and NaHCO₃ (0.984 g, 11.71 mmol, 2 equiv.) was stirred at rt in DMF (10 mL) for 2 hrs. Cyclohexanone (1.15 g, 11.71 mmol) was added and the reaction mixture was stirred for 18 hours. The reaction mixture was then diluted with ethyl acetate (100 mL) and water (30 mL). The organic layer was washed with brine (2×30 mL), dried over MgSO₄, filtered, and concentrated under reduced pressure. Purification of crude fluorohydrins 68 by flash chromatography (25-75% ethyl acetate in hexanes) afforded syn-fluorohydrin 68a (0.92 g, 36% yield) and anti-fluorohydrin 68b (1.21 g, 47% yield) as white solids.

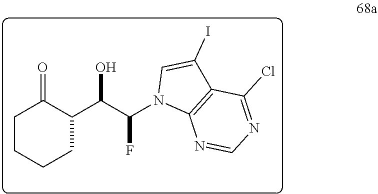

Data for syn-fluorohydrin 68a: ¹H NMR (500 MHz, CDCl₃): δ 8.73 (s, 1H), 8.27 (s, 1H), 7.02 (dd, J=50.0, 5.6 Hz, 1H), 5.82 (d, J=6.9 Hz, 1H), 4.47 (m, 1H), 2.43 (m, 1H), 2.24 (m, 1H), 2.16 (m, 1H), 2.05 (m, 1H), 1.80-1.86 (m, 2H), 1.73 (m, 1H), 1.55-1.60 (m, 2H); ¹³C NMR (125 MHz, CDCl₃): δ 209.9, 151.5, 151.3, 151.0, 134.0, 116.6, 92.5 (d, J=205.2 Hz), 69.7 (d, J=24.4 Hz), 55.3, 51.5, 51.5, 41.5, 29.2, 26.3, 23.5; ¹⁹F NMR (470 MHz, CDCl₃): δ −147.6.

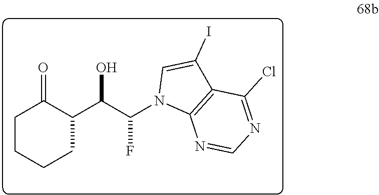

Data for anti-fluorohydrin 68b: ¹H NMR (500 MHz, CDCl₃): δ 8.75 (s, 1H), 8.34 (s, 1H), 7.05 (dd, J=47.6, 7.3 Hz, 1H), 5.59 (d, J=6.7 Hz, 1H), 4.55 (m, 1H), 2.70 (m, 1H), 2.39 (m, 1H), 2.27 (m, 1H), 1.87-1.99 (m, 2H), 1.84 (m, 1H), 1.56-1.76 (m, 3H); ¹³C NMR (125 MHz, CDCl₃): 210.1, 151.6, 151.4, 151.3, 133.8, 116.6, 91.5 (d, J=204.6 Hz), 68.9 (d, J=30.5 Hz), 55.2, 51.1, 41.7, 29.1, 26.4, 23.5

Determination of Relative Stereochemistry for Syn-Fluorohydrin 68a

Fluorohydrin 68a was converted into nucleoside 86. NOE analysis of nucleoside 86 confirmed relative stereochemistry of fluorohydrin 68a.

Determination of Enantiomeric Excess of Fluorohydrin 68a

Using a 1:1 mixture of L-:D-proline, a racemic sample of fluorohydrin 68a was prepared. The enantiomeric fluorohydrins were separated by chiral SFC using Daicel OJ-3; 2900 PSI CO₂, 40° C., 3 ml/min, gradient of 20-30% 25 mM isobutylamine in isopropanol:CO2 over seven minutes; retention times=2.57 min and 2.77 min. The enantiomeric excess of the optically enriched fluorohydrin 68a was determined using the same method (94% ee).

Determination of Enantiomeric Excess of Fluorohydrin 68b

Using a 1:1 mixture of L-:D-proline, a racemic sample of fluorohydrin 68b was prepared. The enantiomeric fluorohydrins were separated by chiral SFC using Daicel OJ-3; 2900 PSI CO₂, 40° C., 3 ml/min, gradient of 1-20% 25 mM diethylamine in methanol:CO₂ over five minutes; retention times=3.10 min and 3.32 min. The enantiomeric excess of the optically enriched fluorohydrin 68b was determined using the same method (93% ee).

Preparation of Aldol Adduct 69

Following General Procedure F, a solution of phthalimidoacetaldehyde (0.050 g, 0.265 mmol), NFSI (0.84 g, 0.265 mmol), L-proline (0.031 g, 0.265 mmol) and 2,6-lutidine (0.031 mL, 0.265 mmol) was stirred at 4° C. in DMF (0.35 mL) for 15 hrs. Thiopyranone 35 (0.307 g, 2.65 mmol) was added and the reaction mixture was stirred for 18 hours. The ratio of diastereomers was determined to be 5:1 by ¹H NMR spectroscopic analysis of the crude product. Purification by flash chromatography (pentane:EtOAc—60:40) afforded an inseparable mixture of syn- and anti-fluorohydrins 69 (0.075 g, 87% yield, d.r.=5:1) as a white solid.

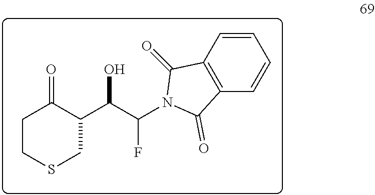

Data for fluorohydrin 69: ¹H NMR (600 MHz, CDCl₃): δ 7.93, 7.92, 7.79, 7.79, 6.26, 6.11, 5.37, 4.78, 3.44, 3.25, 3.24, 3.16, 3.11, 3.09, 3.03, 2.99, 2.98, 2.85, 2.80, 2.79; ¹³C NMR (150 MHz, CDCl₃): δ 212.8, 210.2, 167.1, 167.1, 135.1, 134.9, 131.6, 131.5, 124.3, 124.2, 89.6, 88.3, 70.1, 66.1, 54.6, 53.6, 45.7, 44.9, 34.6, 31.3, 30.7, 30.1; ¹⁹F NMR (470 MHz, CDCl₃): δ −155.5, −158.5 HRMS (EI⁺) calcd for [C₁₅H₁₄FNO₄S+NH₄]⁺ 341.0966; observed 341.0938

Preparation of Aldol Adduct 70

Following General Procedure F, a solution of phthalimidoacetaldehyde (0.050 g, 0.265 mmol), NFSI (0.84 g, 0.265 mmol), L-proline (0.031 g, 0.265 mmol) and 2,6-lutidine (0.031 mL, 0.265 mmol) was stirred at 4° C. in DMF (0.35 mL) for 16 hrs. cyclohexanone (0.275 mL, 2.65 mmol) was added and the reaction mixture was stirred for 18 hours. The ratio of diastereomers was determined to be 5:1 by ¹H NMR spectroscopic analysis of the crude product. Purification by flash chromatography (pentane:EtOAc—60:40) afforded an inseparable mixture of syn- and anti-fluorohydrin 70 (0.068 g, 84% yield, d.r.=5:1) as a white solid.

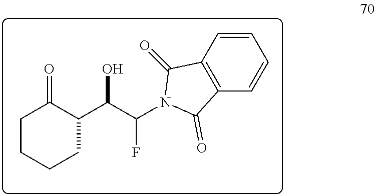

Data for fluorohydrin 70: ¹H NMR (600 MHz, CDCl₃): δ 7.92, 7.91, 7.78, 7.78, 6.29, 6.07, 5.37, 4.63, 3.51, 2.93, 2.92, 2.89, 2.80, 2.44, 2.41, 2.30, 2.25, 2.16, 2.01. 1.99, 1.87, 1.78, 1.71; ¹³C NMR (150 MHz, CDCl₃): δ 215.9, 213.5, 167.1, 167.1, 134.9, 134.8, 131.7, 131.6, 124.1, 124.1, 89.9, 88.3, 69.9, 65.5, 51.8, 51.0, 43.3, 42.7, 32.4, 28.3, 27.8, 26.1, 25.4, 24.8; ¹⁹F NMR (470 MHz, CDCl₃): δ −156.0, −160.7 HRMS (EI⁺) calcd for [C₁₆H₁₇FNO₄]⁺ 306.1136; observed 306.1135

Preparation of Nucleoside Analogue 86

To a suspension of 68a (100 mg, 0.228 mmol) in MeCN (2.0 mL) at 0° C. was added acetic acid (131 μl, 2.285 mmol), followed by sodium triacetoxyborohydride (242 mg, 1.142 mmol). The mixture was stirred at room temperature for 16 h, at which time LCMS indicated complete conversion to the reduced product in approximately 2.5:1 selectivity. The reaction mixture was then diluted with water and ethyl acetate. The organic layer was washed with brine, dried over MgSO₄, filtered, and concentrated under reduced pressure. The crude reduced product was then diluted with MeCN (2.0 mL) and indium chloride (50.5 mg, 0.228 mmol) was added. The resulting reaction mixture was stirred overnight at 50° C. The reaction mixture was then concentrated under reduced pressure and purified by flash column chromatography (25-100% ethyl acetate in hexanes) to afford nucleoside 86 (43 mg, 45%) as a white solid.

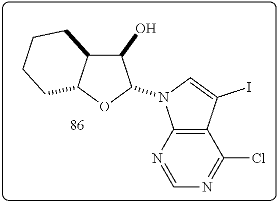

Data for nucleoside analogue 86: [α]_{D}²⁰=−15.0 (c 0.17 in MeOH); IR (neat): ν=3298, 2938, 2852, 1537, 1442, 1204, 1108 cm⁻¹; ¹H NMR (600 MHz, CDCl₃): δ 8.68 (s, 1H), 7.98 (s, 1H), 6.11 (s, 1H), 5.59 (d, J=4.7 Hz, 1H), 4.23 (dd, J=4.7, 4.4 Hz, 1H), 3.64 (ddd, J=11.1, 11.1, 4.0 Hz, 1H), 2.08 (m, 1H), 1.72-1.82 (4H), 1.49 (m, 1H), 1.19-1.40 (m, 3H); ¹³C NMR (150 MHz, CDCl₃): δ 151.1, 150.7, 150.1, 133.3, 116.5, 91.0, 80.9, 76.1, 53.4, 47.7, 40.8, 24.8, 23.6, 23.3 HRMS (EI⁺) calcd for C₁₄H₁₆ClIN₃O₂⁺ 419.9970; Found 419.9952.

Determination of Relative Stereochemistry for Nucleoside 86

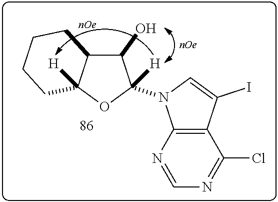

Analysis of 2D NOESY of nucleoside 86 supported the indicated stereochemistry

Preparation of Nucleoside Analogue 87

To a stirred solution of fluorohydrins 70 (0.105 g, 0.344 mmol, 1.0 equiv) in MeCN (3.00 mL) at −15° C. was added tetramethylammoniumtriacetoxyborohydride (0.453 g, 1.72 mmol, 5.0 equiv) and acetic acid (0.190 mL, 3.44 mmol, 10 equiv). The resulting mixture was stirred 16 hours. The reaction mixture was then diluted with a saturated solution of Rochelle salt and washed three times with CH₂Cl₂. The organic layer was separated, dried over MgSO₄, filtered, and concentrated under reduced pressure. The crude product S70 was purified by flash chromatography (EtOAc:pentane—70:30) to afford S70 as a white solid (0.076 g, 72%)

To a stirred solution of syn-diol-fluorohydrins S70 (0.076, 0.248 mmol, 1.0 equiv.) in MeCN (2.50 mL) was added InCl₃ (0.014 g, 0.062 mmol, 0.25 equiv.) and the reaction mixture was stirred for 24 hours. The reaction mixture was diluted with CH₂Cl₂ and was washed with saturated sodium bicarbonate solution. The organic layer was separated, dried over MgSO₄, filtered, and concentrated under reduced pressure. The ratio of anomers (α:β) was determined to be 2.5:1 by ¹H NMR spectroscopic analysis of the crude product. The crude product 87 was purified by flash chromatography (EtOAc:pentane—25:75) to afford nucleoside 87 (α-anomer) as a colorless oil (42.7 mg, 60%)

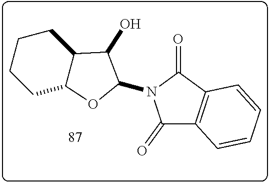

Data for nucleoside analogue 87 (α-anomer): [α]_{D}²⁰=+46.6 (c 0.38 in CH₂Cl₂); IR (neat): ν=3475, 2935, 1708, 1370, 720 cm⁻¹; ¹H NMR (600 MHz, CDCl₃): δ 7.88 (m, 2H), 7.77 (m, 2H), 6.13 (d, J=5.0 Hz, 1H), 4.40 (ddd, J=11.8, 5.0, 4.8 Hz, 1H), 4.03 (ddd, J=10.6, 10.6, 4.1 Hz, 1H), 3.13 (d, J=11.9 Hz, 1H), 2.22 (m, 1H), 1.94 (m, 1H), 1.85 (m, 2H), 1.62 (dddd, J=11.9, 11.9, 4.6, 3.2 Hz, 1H), 1.51 (m, 1H), 1.23-1.40 (3H); ¹³C NMR (150 MHz, CDCl₃): δ 169.1, 134.6, 132.1, 123.8, 84.4, 81.1, 75.3, 51.4, 31.7, 25.4, 24.0, 24.0 HRMS (EI⁺) calcd for C₁₆H₁₈NO₄ [M+H⁺] 288.1230; found 288.1246

Determination of Relative Stereochemistry for Nucleoside 87

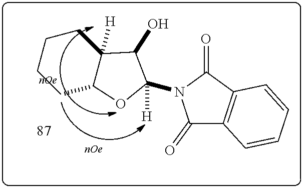

Analysis of 2D NOESY of nucleoside 87 (α-anomer) supported the indicated stereochemistry

Preparation of Nucleoside Analogue 88

To a stirred solution of fluorohydrins 69 (0.097 g, 0.30 mmol, 1.0 equiv) in MeCN (3.00 mL) at −15° C. was added tetramethylammoniumtriacetoxyborohydride (0.395 g, 1.50 mmol, 5.0 equiv) and acetic acid (0.172 mL, 1.50 mmol, 10 equiv). The resulting mixture was stirred 16 hours. The reaction mixture was then diluted with a saturated solution of Rochelle salt and washed three times with CH₂Cl₂. The organic layer was separated, dried over MgSO₄, filtered, and concentrated under reduced pressure. The crude product S69 was purified by flash chromatography (EtOAc:pentane—70:30) to afford S69 as a white solid (0.068 g, 70%)

To a stirred solution of syn-diol-fluorohydrins S69 (0.047, 0.143 mmol, 1.0 equiv.) in MeCN (1.43 mL) was added InCl₃ (7.9 mg, 0.036 mmol, 0.25 equiv.) and the reaction mixture was stirred for 24 hours. The reaction mixture was diluted with CH₂Cl₂ and was washed with saturated sodium bicarbonate solution. The organic layer was separated, dried over MgSO₄, filtered, and concentrated under reduced pressure. The ratio of anomers (α:β) was determined to be 3:1 by ¹H NMR spectroscopic analysis of the crude product. The crude product 88 was purified by flash chromatography (EtOAc:pentane—40:60) to afford 88 (α-anomer) as a colorless oil (23.7 mg, 73%).

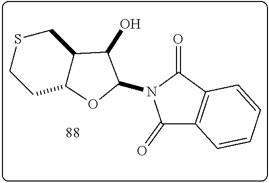

Data for nucleoside analogue 88 (α-anomer): [α]_{D}²⁰=+18.6 (c 2.37 in CH₂Cl₂); IR (neat): ν=3475, 2923, 1774, 1709, 1373, 719 cm⁻¹; ¹H NMR (600 MHz, CDCl₃): δ 7.88 (m, 2H), 7.77 (m, 2H), 6.13 (d, J=4.9 Hz, 1H), 4.40 (ddd, J=11.5, 4.7, 4.7 Hz, 1H), 4.03 (ddd, J=11.2, 11.2, 3.6 Hz, 1H), 3.35 (d, J=11.9 Hz, 1H), 2.98 (dd, J=13.1 11.9 Hz, 1H), 2.82 (m, 2H), 2.69 (m, 1H), 2.50 (m, 1H), 2.10 (m, 1H), 1.74 (m, 1H); ¹³C NMR (150 MHz, CDCl₃): δ 169.2, 134.8, 131.9, 124.0, 83.0, 80.2, 75.2, 51.3, 33.5, 27.6, 27.4. HRMS (EI⁺) calcd for C₁₅H₁₉N₂O₄S [M+NH₄+] 323.1060; found 323.1037

Determination of Relative Stereochemistry for Nucleoside 88

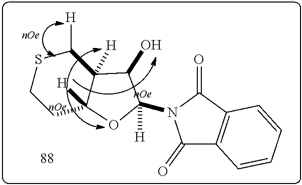

Analysis of 2D NOESY of nucleoside 88 (α-anomer) supported the indicated stereochemistry.

J-Based Configurational Analysis (JBCA)

The fluorine stereoconfigurations of the following compounds were assigned using NMR J-based configuration analysis, and then the assignments were verified using density functional theory calculations. The other stereocenters were known based on synthesis.

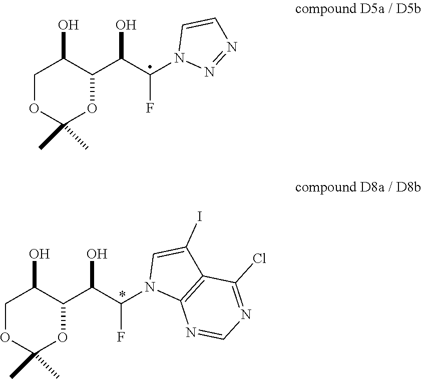

NMR Spectroscopy

NMR samples were prepared by dissolving several mg in 0.75 mL of DMSO-d₆. These solutions were then transferred to 5-mm NMR tubes. Proton chemical shifts were referenced to residual DMSO-d₅ at 2.50 ppm, and carbon chemical shifts were referenced to DMSO-d₆ at 39.52 ppm. NMR spectra were acquired on either a 600 MHz Bruker AVANCE III HD spectrometer equipped with a 5-mm triple resonance (HCN) helium cryoprobe or a 500 MHz Bruker AVANCE III HD spectrometer equipped with a 5-mm inverse Prodigy probe. Data were processed using Mnova, version 12.0.4. ¹H, ¹³C, COSY, HSQC, and HMBC data were acquired for all compounds to assign the proton and carbon chemical shifts. Either NOESY or ROESY spectra were acquired using a 200 ms mixing time to aid in the stereochemical determinations.

DFT Calculations

Density functional theory (DFT) calculations of NMR parameters, chemical shifts (d, ppm) and coupling constants (J, Hz), were performed in order to verify the peak assignments and relative stereoconfiguration. Initially, an ensemble of conformers was generated using a mixed torsional/low-mode sampling search with the OPLS3e force field, as implemented in Macromodel (52). The set of conformers less than 5 kcal/mol were then further subjected to DFT geometry optimizations and frequency determinations (to verify potential energy minima) using the B3LYP/6-31G(d) model chemistry in Gaussian '16 (53). Isotropic magnetic shielding values, s, were then calculated starting from the optimized geometries using either WPO4/cc-pVDZ or wB97X-D/6-31 G(d,p) gauge-including atomic orbital (GIAO) methods for proton and carbon, respectively, with implicit solvent corrections from the polarized continuum model (PCM). Linear scaling factors [d=intercept−s/−slope]were applied to convert the s values to chemical shifts, d, in ppm. The scaling factors were previously determined from a large test set of known structures, curated by Rablen et. al. (54) and Lodewyk et. al. (55) (¹H: intercept=31.8465, slope=−0.9976; ¹³C: intercept=198.1218, slope=−0.9816). Coupling constants were calculated using the B3LYP/6-31 G(d) model chemistry. Gibbs free energies were calculated using M06-2X/6-31+G(d,p) with SMD solvation model, and both chemical shifts and coupling constants were weighted according to the Boltzmann energy distribution.

Single Crystal X-Ray Diffraction

Suitable crystals were suspended in paratone oil, mounted on a MiTeGen Micro Mount, and transferred to the X-ray diffractometer, which was set to 150 K using an Oxford Cryosystems Cryostream. Data was collected at 150 K on a Bruker Smart instrument equipped with an APEX II CCD area detector fixed at a distance of 5.0 cm from the crystal and a Cu Kα fine focus sealed tube (λ=1.54178 Å) operated at 1.5 kW (45 kV, 0.65 mA), filtered with a graphite monochromator. Data were collected and integrated using the Bruker SAINT software package and were corrected for absorption effects using the multi-scan technique (SADABS) (56). The structures were solved with direct methods (SIR92) and subsequent refinements were performed using SHELXL (57) and ShelXIe (58). Hydrogen atoms on carbon atoms were included at geometrically idealized positions (C—H bond distance 0.95 Å) and were not refined. The isotropic thermal parameters of the hydrogen atoms were fixed at 1 0.2 times that of the preceding carbon atom. Diagrams were prepared using Mercury (59) and POV-RAY (60). Table 1 shows the summary of XRD analysis.

TABLE 1
Summary of XRD analysis

| Compound Reference | Bis-PNB ester of 18a | | D9a | | D7b | |
|---|---|---|---|---|---|---|
| Chemical Formula | C₂₅H₂₃N₄O₁₀F | | C₁₂H₁₆FIN₂O₆ | | C₁₆H₁₈O₆FN | |
| Formula Mass | 558.47 | | 430.1709 | | 339.31 | |
| Crystal System | | | Triclinic | | | |
| a/Å | 16.7932 | (13) | 9.2762 | (4) | 7.9861 | (18) |
| b/Å | 15.8691 | (11) | 9.6024 | (4) | 8.252 | (3) |
| c/Å | 19.4773 | (14) | 9.8870 | (4) | 12.936 | (3) |
| α/° | 90 | | 69.8990 | (10) | 79.83 | (2) |
| β/° | 90 | | 64.8030 | (10) | 81.342 | (19) |
| γ/° | 90 | | 87.7980 | (10) | 89.66 | (2) |
| Unit cell volume/Å³ | 5190.6 | (7) | 742.28 | (5) | 829.4 | (4) |

| | | | | | |
|---|---|---|---|---|---|
| Temperature/K | 150 | (2) | 100.15 | 150 | (2) |

| Space group | Pbca | P-1 | P1 |
|---|---|---|---|
| Number of formula unit | 8 | 2 | 2 |
| per cell/Z | | | |
| Radiation type | Cu Kα | | Cu Kα |

| Absorption coefficient, | 1.001 | 17.367 | 0.951 | |
|---|---|---|---|---|
| μ/mm⁻¹ | | | | |
| No. of reflections | 4759 | 18953 | 4704 | |
| Flack parameter | — | — | −0.4 | (3) |

| | | | |
|---|---|---|---|
| Rᵢₙₜ | 0.0309 | 0.0383 | 0.0764 |
| Final R₁ values (I > 2σ(I)) | 0.0642 | 0.0246 | 0.0693 |
| Final wR(F²) values | 0.1932 | 0.0632 | 0.1717 |
| (I > 2σ(I)) | | | |
| Final R₁ values (all data) | 0.0711 | 0.0246 | 0.0846 |
| Final wR(F²) (all data) | 0.2018 | 0.0632 | 0.1846 |
| Goodness of fit | 1.050 | 1.116 | 1.021 |

Examples of Large-Scale Preparation of αFAR Products

No additional optimization of the reaction conditions was done for large scale synthesis and in most cases only select chromatographed fractions were included in the final mass.

Large-Scale Preparation of 55

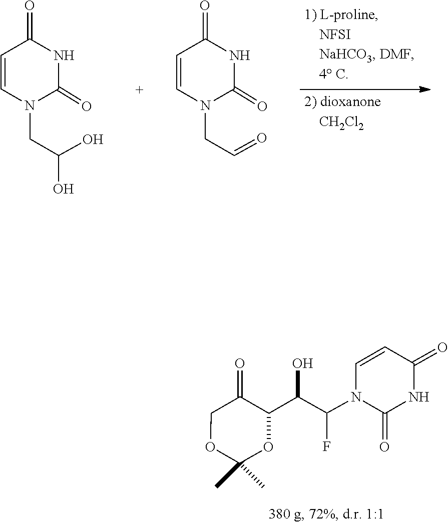

Three reactions were ran in parallel. To a large reactor was charged DMF (2.1 L) and uracil (300.0 g, 2.68 mol, 1.0 equiv.) at 15-25° C. Then, the reactor was individually charged with DBU (807 mL, 5.35 mol, 2.0 equiv.) and 2-bromo-1,1-diethoxy-ethane (483 mL, 3.21 mol, 1.2 equiv.). The reaction mixture was heated to 90° C.-100° C. for 16 hrs. The reaction mixture cooled to 25° C. and the three batches were combined and concentrated to dryness to give a residue. To the residue was water (2.5 L) and the pH of the resulting mixture was adjusted with 1 M HCl to 6-7 and extracted with EtOAc (2.0 L×8). The combined organic layer was dried with Na₂SO₄, filtered and the filtrate was concentrated to dryness under reduced pressure to give a residue. The crude residue was triturated with MBTE (3 L) at 20° C. for 60 minutes. The crude residue was purified by silica gel chromatography (petroleumether:EtOAc:CH₂Cl₂=10:2:1). The alkylated uracil product (738 g, 3.23 mol, 40.3% yield) was isolated as a white solid.

To a large reactor was charged HCl (1 M, 2.89 L, 1.0 equiv.) and the alkylated thymine product (660 g, 2.89 mol, 1.0 equiv.) at 15-25° C. The reaction mixture was heated to 90˜100° C. and stirred for 3 hours. Following complete consumption of starting material, the reaction mixture was cooled to 0° C. and stirred for 30 minutes. The resulting suspension was filtered, dried, and the crude product was used in the next step without further purification. The aldehyde/hydrate (425 g, 2.76 mol, 95.4%) was obtained as an off-white solid.

To a large reactor was charged with DMF (2800 mL) and aldehyde (400 g, 2.60 mol, 1.0 eq) and the resulting mixture was cooled to 4° C. Then, the reactor was individually charged with NFSI (818 g, 2.60 mol, 1.0 equiv.), NaHCO₃ (218 g, 2.60 mol, 1.0 equiv.) and L-proline (299 g, 2.60 mol, 1.0 equiv.). The reaction mixture was stirred at 4° C. for 18 hrs. HPLC (ET24077-13-P1A) showed starting material (RT=0.34) was consumed completely. To the reaction mixture was added dropwise a solution of dioxanone (226 g, 1.74 mol, 0.67 eq) in CH₂Cl₂ (1.3 L) at 4° C. The reaction mixture was stirred at 15˜25° C. for 18 hrs. HPLC (ET24077-13-P1A) showed starting material (RT=1.72 min) showed the α-fluorohydrate was completely consumed. 14.0 L H₂O was added into the reaction mixture and extracted with EtOAc (3.0 L×8). The organic phase was dried with Na₂SO₄, then filtered, and the filtrate was concentrated to dryness under reduced pressure to give a residue. The residue was purified by flash silica gel chromatography (Eluent of 0˜50% ethyl acetate/petroleum ether gradient) to afford 55 as a yellow oil (380 g, 72% yield, d.r. 1:1).

Large-Scale Preparation of A3

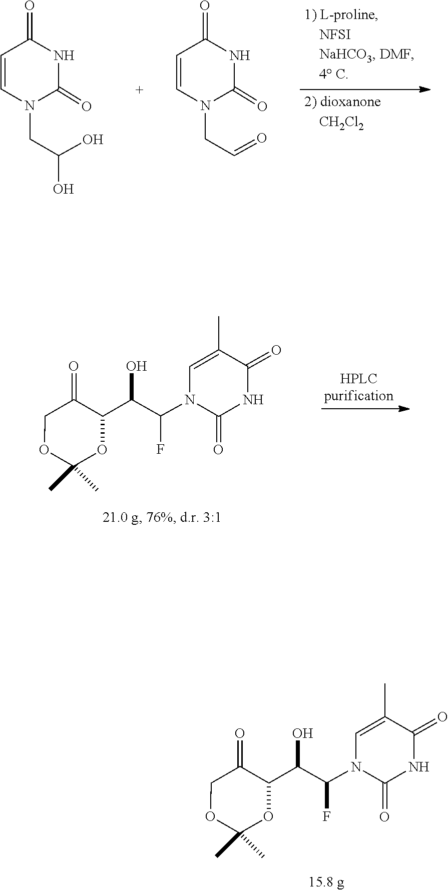

To a large reactor was charged DMF (1.7 L) and thymine (85.0 g, 0.674 mol, 1.0 equiv.) at 15-25° C. Then, the reactor was individually charged with DBU (203 mL, 1.35 mol, 2.0 equiv.) and 2-bromo-1,1-diethoxy-ethane (122 mL, 0.809 mol, 1.2 equiv.). The reaction mixture was heated to 90° C. for 14.5 hrs. The reaction mixture was concentrated to dryness to give a residue. To the residue was added EtOAc (1.7 L) and water (1.7 L), the organic layer was separated, the aqueous layer was extracted with EtOAc (1.7 L×2). The combined organic layer was washed with brine (500 mL), dried with Na₂SO₄, filtered and the filtrate was concentrated to dryness under reduced pressure to give a residue. The residue was purified by flash silica gel chromatography (ISCO®; 5000 g SepaFlash® Silica Flash Column, Eluent of 30˜60% Ethyl acetate/Petroleum ether gradient@800 mL/min). The alkylated thymine product (80.0 g, 301 mmol, 22.4% yield, 91.3% purity) was obtained as an off-white solid.

To a large reactor was charged HCl (1 M, 330 mL, 1.0 equiv.) and the alkylated thymine product (80.0 g, 0.330 mol, 1.0 equiv.) at 15-25° C. The reaction mixture was heated to 90˜100° C. and stirred for 15 hours. HPLC (ET17680-15-P1A) indicated starting material (RT=2.77) was consumed completely. The mixture was concentrated to dryness and the crude product was used in the next step without further purification. The aldehyde/hydrate (63.0 g mixture) was obtained as an off-white solid.

To a large reactor was charged with DMF (190 mL) and aldehyde (0.131 mol, 1.0 eq) and the resulting mixture was cooled to 4° C. Then, the reactor was individually charged with NFSI (41.3 g, 0.131 mol, 1.0 equiv.), NaHCO₃ (11.0 g, 0.131 mol, 1.0 equiv.) and L-proline (15.1 g, 0.131 mol, 1.0 equiv.). The reaction mixture was stirred at 4° C. for 18.5 hrs. HPLC (ET17918-3-P1A) showed starting material (RT=1.99) was consumed completely. To the reaction mixture was added dropwise a solution of dioxanone (11.4 g, 0.088 mol, 0.67 eq) in CH₂Cl₂ (200 mL) at 4° C. The reaction mixture was stirred at 15˜25° C. for 20.5 hrs. 570 mL CH₂Cl₂ was added into the mixture, and the organic phase was washed with water (190 mL×3). The organic phase was dried with Na₂SO₄, then filtered, and the filtrate was concentrated to dryness under reduced pressure to give a residue. The residue was purified by flash silica gel chromatography (ISCO®; 330 g SepaFlash® Silica Flash Column, Eluent of 0˜100% ethyl acetate/petroleum ether gradient@200 mL/min) to afford A3 as a yellow oil (21.0 g, 76% yield, d.r. 3:1 (syn:anti)).

### 16 g Scale Preparation of 59

39.0 g of A3 was dissolved in 240 mL of ethyl acetates and repurified by prep-HLPC to give 18.0 g of product. The 18.0 g product was dissolved in 240 mL of CH₂Cl₂ and concentrated under reduced pressure to give 17.5 g of 59. The 17.5 g of 59 was freeze-dried to obtain 15.8 g of 59 as a white solid (94.3% purity).

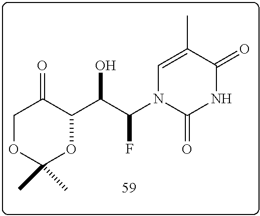

Data for syn-fluorohydrin 59: [α]_{D}²⁰=−89.4 (c 1.1 in MeOH); IR (neat): ν=2993, 1694, 1450, 1369, 1082, 1045 cm⁻¹; ¹H NMR (400 MHz, CDCl₃): δ 8.30 (br s, 1H), 7.57 (dd, J=1.3, 1.2 Hz, 1H), 6.66 (ddd, J=42.7, 2.3, 1.3 Hz, 1H), 4.40 (dd, J=8.9, 1.4 Hz, 1H), 4.33 (dd, J=17.7, 1.4 Hz, 1H), 4.12 (d, J=17.7 Hz, 1H), 4.10 (ddd, J=15.4, 3.1, 2.3 Hz, 1H), 3.64 (d, J=3.0 Hz, 1H), 1.95 (d, J=1.2 Hz, 3H), 1.52 (s, 3H), 1.46 (s, 3H); ¹³C NMR (100 MHz, CDCl₃): δ 211.2, 163.2, 149.9, 137.1 (d, J=4.0 Hz), 111.0, 102.1, 90.2 (d, J=207.8 Hz), 71.6 (d, J=2.3 Hz), 70.9 (d, J=23.4 Hz), 66.5, 23.8, 23.4, 12.6; ¹⁹F NMR (470 MHz, CDCl₃): δ −177.8 HRMS (EI⁺) calcd for C₁₃H₁₈FN₂O₆[M+H]⁺ 317.2929; found 317.1142

Large-Scale Preparation of A5

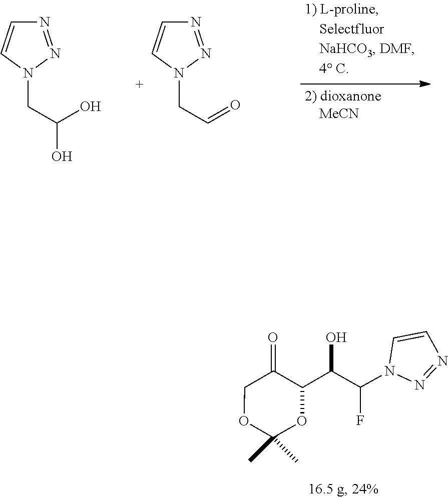

This reaction was executed without further optimization. Crude A5 was purified by column chromatography to afford 16.5 g of A5 (impure fractions were discarded).

Large-Scale Preparation of A6

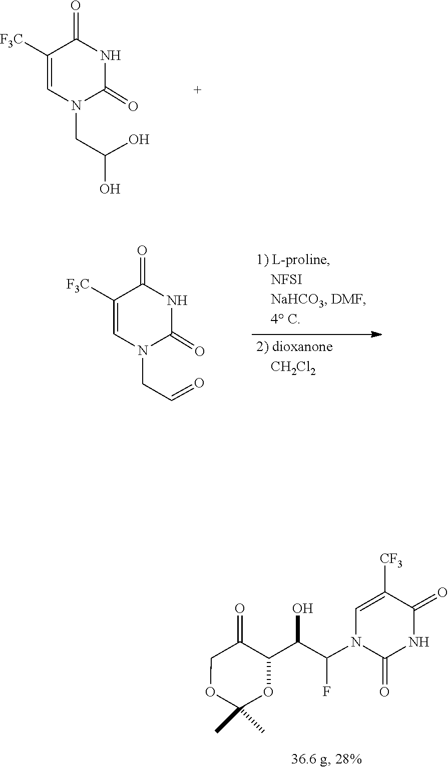

The reaction was executed without further optimization. The reaction was stopped after only 16 hrs. Crude A6 was purified by prep-HPLC to afford 36.6 g of A6 (impure fractions were discarded).

Large-Scale Preparation of A8

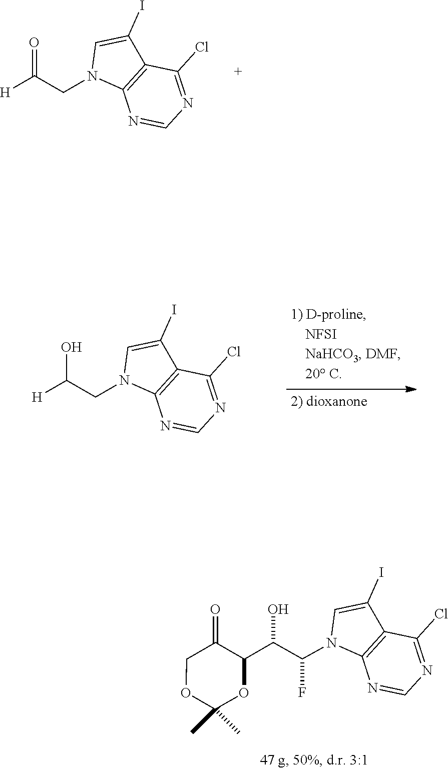

The reaction was executed without further optimization. Crude A8 was purified by prep-HPLC to afford 47 g of A8 (impure fractions were discarded).

Development of a Short De Novo NA Synthesis.

We investigated the α-fluorination (33) of α-pyrazolyl aldehyde 15 (FIG. 2B) and found that a combination of L-proline and N-fluorobenzene-sulfonamide (NFSI) in DMF(34) provided an α-fluorohydrate as the sole product (Table 2).

TABLE 2
Optimization of αFAR for α-pyrazole aldehyde

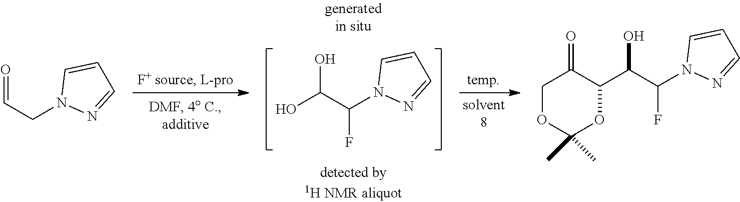

| entry | temp. (° C.) | additive^{a} | F⁺ source^{a} | solvent^{b} | e.r. | yield |
|---|---|---|---|---|---|---|
| 1 | 20 | NaHCO₃ | NFSI | CH₂Cl₂^{c} | N.D. | <10% |
| 2 | 20 | NaHCO₃ | NFSI | CH₂Cl₂ | (86:14); | 72% |
| | | | | | (99:1) | |
| 3 | 4 | NaHCO₃ | NFSI | CH₂Cl₂ | N.D. | 23% |
| 4 | 20 | NaHCO₃ | NFSI | MeCN | (93:7); (98:2) | 64% |
| 5 | 20 | NaHCO₃ | NFSI | DMF | (91:9); (98:2) | 56% |
| 6 | 20 | None | NFSI | CH₂Cl₂ | N.D. | <10% |
| 7 | 20 | NaHCO₃ | Selectfluor | MeCN | N.D. | 19% |
| 8 | 20 | None | Selectfluor | CH₂Cl₂ | N.D. | <5% |
| 9 | 20 | NaHCO₃ | Selectfluor | CH₂Cl₂ | N.D. | 25% |

^{a}1.5 equiv.
^{b}vol. of solvent added was 1.25 × DMF vol. in 1^{st} step.
^{c}volume of solvent added was 9 × DMF vol. added in 1^{st} step.

The direct addition of dioxanone 8 in MeCN to the reaction mixture afforded the fluorohydrins 16a and 16b in good yield and enantioselectivity (FIG. 2B, entry 2). As indicated, the fluorohydrins 16a and 16b were formed as a ˜1.4:1 mixture of epimers at the pseudo anomeric carbon (indicated with *) that do not interconvert under the reaction or isolation/purification conditions.

Reduction of the fluorohydrins 16a and 16b provided a mixture of 1,3-syn diols that was then treated with one of several Lewis acids to promote displacement of the fluoride by the distal alcohol function and an AFD reaction using fluorophilic Sc(OTf)₃(36) was realized that afforded the NA 17 in 38% yield as a single β-anomer (FIG. 2B, entry 4). Additionally, we found that treatment of a mixture of the diols 12a and 12b with base (NaOH) resulted in the formation of a mixture of α- and β-anomeric NAs that varied in composition depending on reaction time and equivalents of base (FIG. 2B, entries 5 and 6). Using a large excess of NaOH (10 equiv, entry 6), the β-anomer 17 was formed as the exclusive product in excellent yield (76%). To further examine the mechanism of cyclization, the intermediate diols 18a and 18b were separated by flash column chromatography and their relative stereochemistry assigned by J-based configurational analysis and/or X-ray analysis of derivatives.

Subjecting the purified syn-fluorohydrin 18a to the AFD reaction (NaOH, CH-3CN, FIG. 2C) promoted a clean cyclization to the β-anomer 17 via an S_{N}2 process. Similarly, the anti-fluorohydrin 18b cyclized to afford the α-anomer 19, again via stereochemical inversion. Under these same reaction conditions, the α-anomer 19 epimerizes to afford the naturally configured β-anomer 17, and thus both fluorohydrin aldol products can be transformed together into a single naturally configured p-D-NA. The enantiomeric purity of the NA 17 (e.r.=95:5, FIG. 2B, entry 6) represents an average of the enantiomeric purities of the epimeric fluorohydin αFAR products 16.

Preparation of NAs Using αFAR and AFD Strategies.

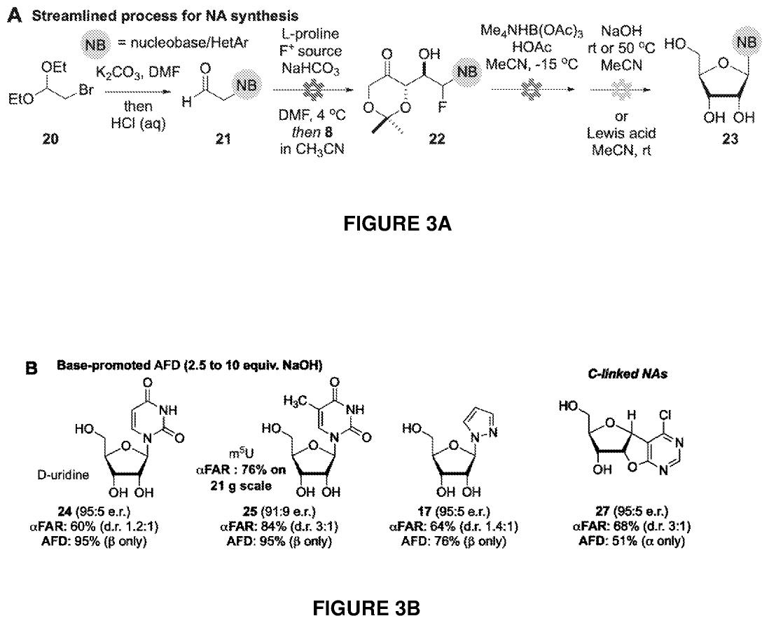

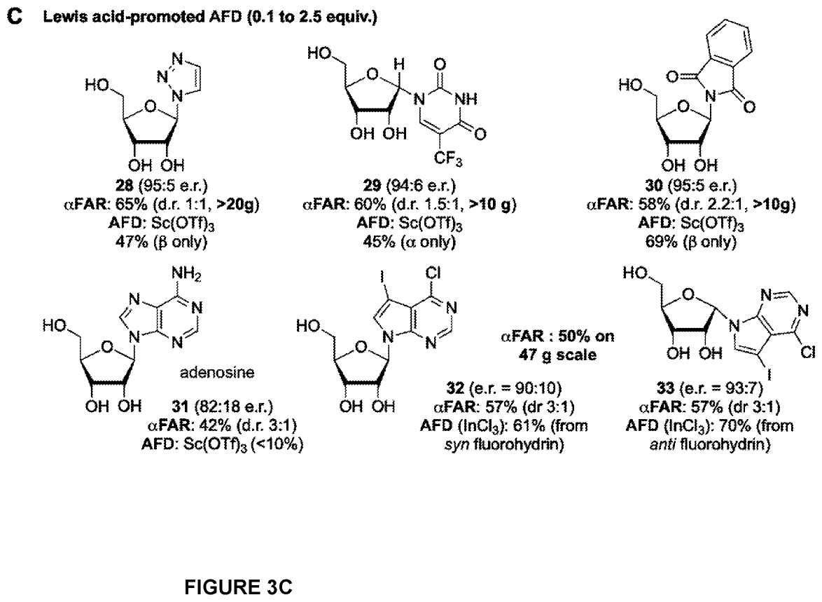

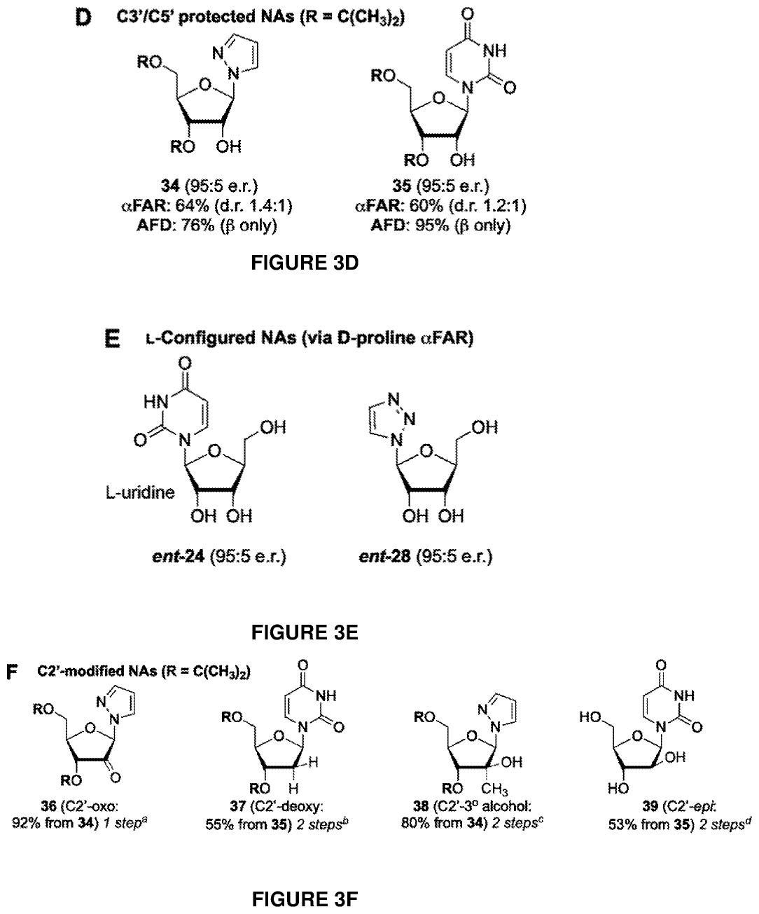

We prepared a collection of acetaldehyde derivatives through the alkylation of several heterocycles with bromoacetaldehyde diethyl acetal (FIG. 3A). Using either Selectfluor or NFSI as the electrophilic fluorinating agent (F⁺), the resulting aldehydes 21 then underwent proline-catalyzed αFAR with dioxanone 8 to provide a collection of fluorohydrin aldol products 22 functionalized with one of the heterocycles uracil, thymine, triazole, deazadenine, pyrazole, phthalimide, adenine, 2,6-dichloropyrimidine or tetrazole. These fluorohydrins were generally produced in good to excellent yield and enantiomeric purity. Table 3 shows optimization of αFAR for α-(1, 2, 3)-triazole aldehyde.

TABLE 3
Optimization of αFAR for α-(1,2,3)-triazole aldehyde

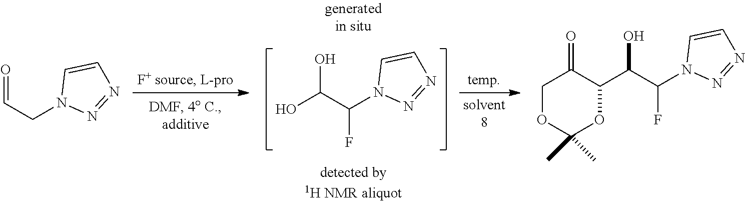

| entry | temp. (° C.) | additive^{a} | F⁺ source^{a} | solvent^{b} | e.r. | yield |
|---|---|---|---|---|---|---|
| 1 | 20 | NaHCO₃ | NFSI | CH₂Cl₂^{c} | N.D. | <5% |
| 2 | 20 | NaHCO₃ | Selectfluor | CH₂Cl₂ | (67:34); (95:5) | 54% |
| 3 | 20 | NaHCO₃ | Selectfluor | DMF | (91:9); (96:4) | 41% |
| 4 | 20 | NaHCO₃ | Selectfluor | THF | (80:20); (N.D.) | 58% |
| 5 | 20 | NaHCO₃ | Selectfluor | MeCN | (94:6); (96:4) | 65% |
| 6 | 4 | NaHCO₃ | Selectfluor | MeCN | N.D. | 29% |
| 7 | 37 | NaHCO₃ | NFSI | CH₂Cl₂ | N.D. | 90% |
| 8 | 20 | None | Selectfluor | MeCN | N.D. | <10% |

^{a}1.5 equiv.
^{b}vol. of solvent added was 1.25 × DMF vol. in 1^{st} step.
^{c}volume of solvent added was 9 × DMF vol. added in 1^{st} step

In the case of the adenine containing fluorohydrin, the enantiomeric purity was lowered by competing (non-proline) catalysis in the αFAR. Each of the αFAR products was isolated as a mixture of epimers at the fluoromethine center that subsequently underwent a 1,3-syn selective carbonyl reduction and AFD promoted by either base (NaOH, FIG. 3B) or a Lewis acid (FIG. 3C) as indicated. Several heterocycles were compatible with this process (FIGS. 3B-E) and uracil, thymine or adenine-substituted acetaldehydes could be exploited in short (4 step total) de novo syntheses of the endogenous ribonucleosides uridine (U: 24), 5-methyluridine (m⁵U: 25) and adenosine (A: 31). In these studies, Lewis acids for promoting AFD reactions were InCl₃ or Sc(OTf)₃, while pyrazole- and uracil-derived fluorohydrins were cyclized using NaOH. In this study, with the exception of triazole 28, trifluoromethyluracil 29 and deazaadenines 32 and 33, the NAs were produced as an approximate average of the enantiomeric purities of the individual precursor fluorohydrin epimers 22. Thus, the majority of NAs underwent epimerization following AFD providing a straightforward means to convert the mixture of epimeric aldol products into a single, naturally configured p-D-nucleoside analogue. For the trifluoromethyl uracil 29 and deazaadenines 32 and 33, αFAR products (e.g., 22) were reduced, separated and treated individually with Sc(OTf)₃ or InCl₃. As indicated in FIG. 3C, for trifluoromethyl uracil, only the anti-fluorohydrin underwent AFD to form 29, which did not epimerize under the reaction conditions. In the case of the deazaadenine, both the syn-fluorohydrin and anti-fluorohydrin underwent AFD to provide the β- and α-anomers 32 and 33, respectively, confirming that these reactions proceed via direct fluoride displacement.

Several of the αFARs were demonstrated on >10 g scale (e.g., 25, 28, 29, 30 and 32 (FIG. 3C) and we noted an improvement in diastereoselectivity when reactions were executed on larger scale. We also found that the C-linked NA 27 could be prepared using this sequence of reactions starting from a dichloropyrimidine, further extending the utility of this strategy to an additional and important class of NAs.(37) Here, the major product of the αFAR was an anti-fluorohydrin, which cyclizes stereospecifically to α-D-nucleoside analogue and undergoes a second cyclization event under the reaction conditions to form the tricycle 27. In addition to naturally configured NAs, this strategy can be easily adapted for the synthesis of enantiomeric (L-configured) nucleosides and NAs (FIG. 3E) by using D-proline in the αFAR. Thus, L-uridine (ent-24) and the L-configured NA ent-28 were accessed in this straightforward manner. While crude reaction mixtures were generally treated with aqueous acid to remove the acetonide protecting group and enable isolation of the targeted NA, eliminating this step allowed us to isolate C3′/C5′-protected NAs directly (e.g., 34 and 35, FIG. 3D). To demonstrate that these acetonide-protected NAs can be further derivatized using standard protocols, several C2′-modified NAs were prepared, including C2′-oxo (36), C2′-deoxy (37), C2′−3° alcohol (38) and C2′-epi (39) (FIG. 3F).

Optimization of AFD Reactions are shown in Tables 4 and 5.

TABLE 4
Optimization of AFD reaction

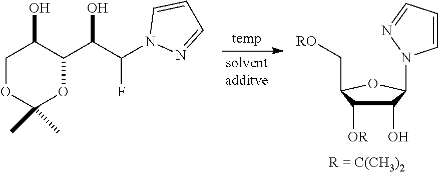

| entry | temp. (° C.) | additive | solvent^{a} | β:α | yield |
|---|---|---|---|---|---|
| 1 | 20 | NaOH | EtOH | — | <10% |
| 2 | 20 | NaOH^{b} | MeCN | 1:1 | 72% |
| 3 | 50 | NaOH^{c} | MeCN | 1:0 | 76% |
| 4 | 20 | TMSOTf | MeCN | — | 0% |
| 5 | 20 | Sc(OTf)₃ | MeCN | 0:1 | 38% |
| 6 | 20 | TsOH | MeCN | — | 0% |
| 7 | 100 | NaHCO₃ | Toluene | — | <10% |

^{a}0.10M.
^{b}2.5 equiv.
^{c}10 equiv.

TABLE 5
Optimization of AFD reaction.

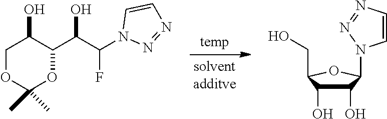

| entry | temp. (° C.) | additive | solvent^{a} | β:α | yield |
|---|---|---|---|---|---|
| 1 | 20 | NaOH | MeCN | — | 0% |
| 2 | 50 | NaOH^{b} | MeCN | — | 0% |
| 3 | 20 | Sc(OTf)₃^{c} | MeCN | — | 0% |
| 4 | 20 | Sc(OTf)₃^{c} | CH₂Cl₂ | — | 0% |
| 5 | 20 | InCl₃ | MeCN | — | 0% |
| 6 | 20 | TMSOTf | CH₂Cl₂ | — | 0% |
| 7 | 20 | Sc(OTf)₃^{d} | MeCN | 1:0 | 21% |
| 8 | 20 | NaOH | EtOH | — | 0% |
| 9 | 20 | Sc(OTf)₃^{e} | MeCN | 1:0 | 47% |

^{a}0.10M.
^{b}10 equiv.
^{c}0.15 equiv.
^{d}1.5 equiv.
^{e}2.5 equiv.

Rapid Synthesis of C4′-Modified α-L-Configured NAs.

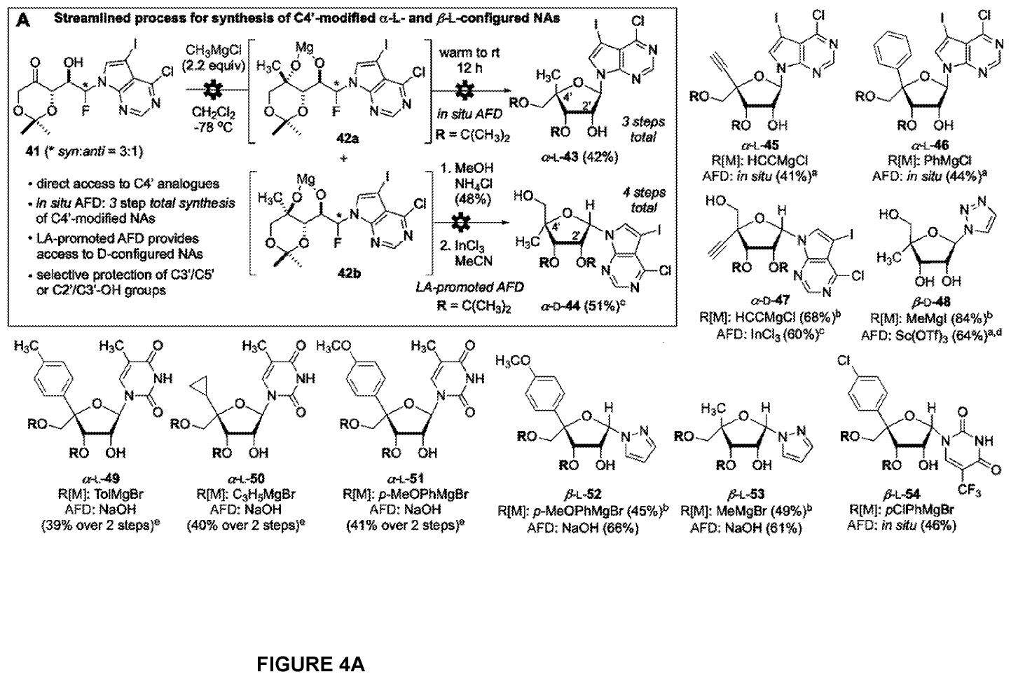

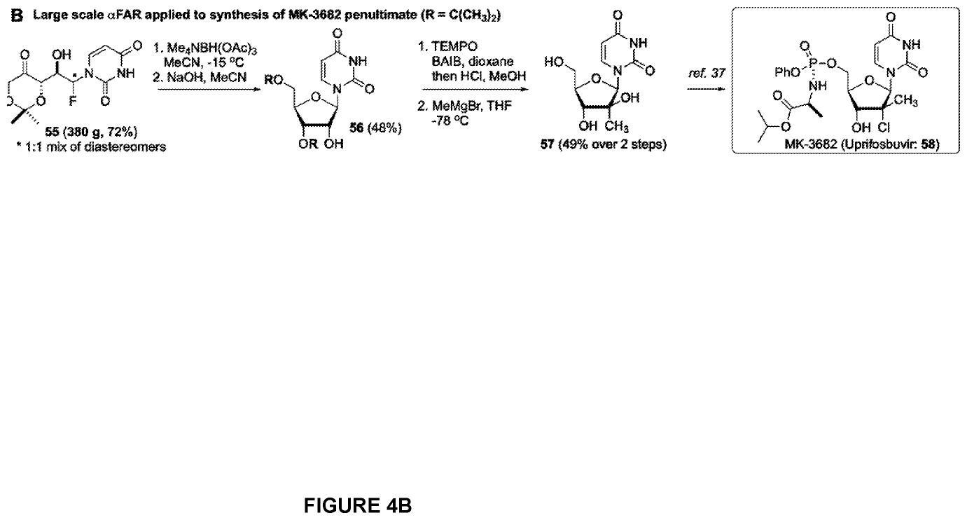

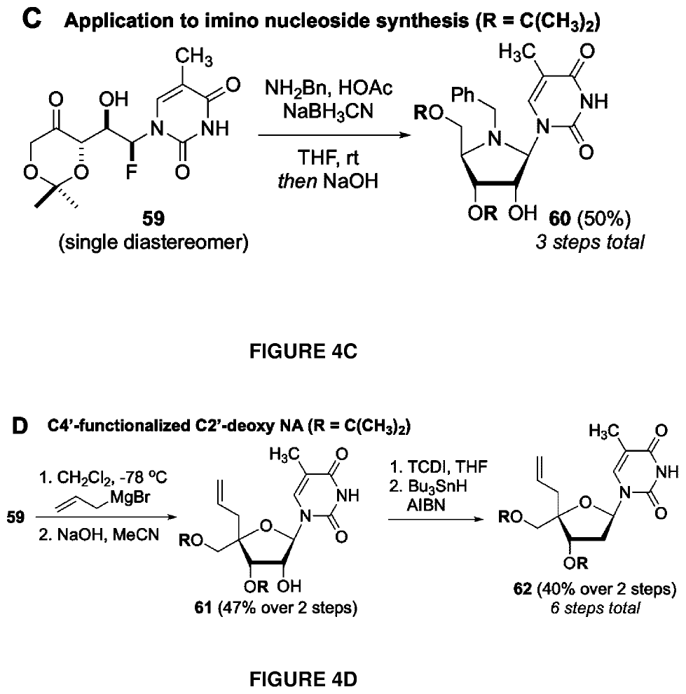

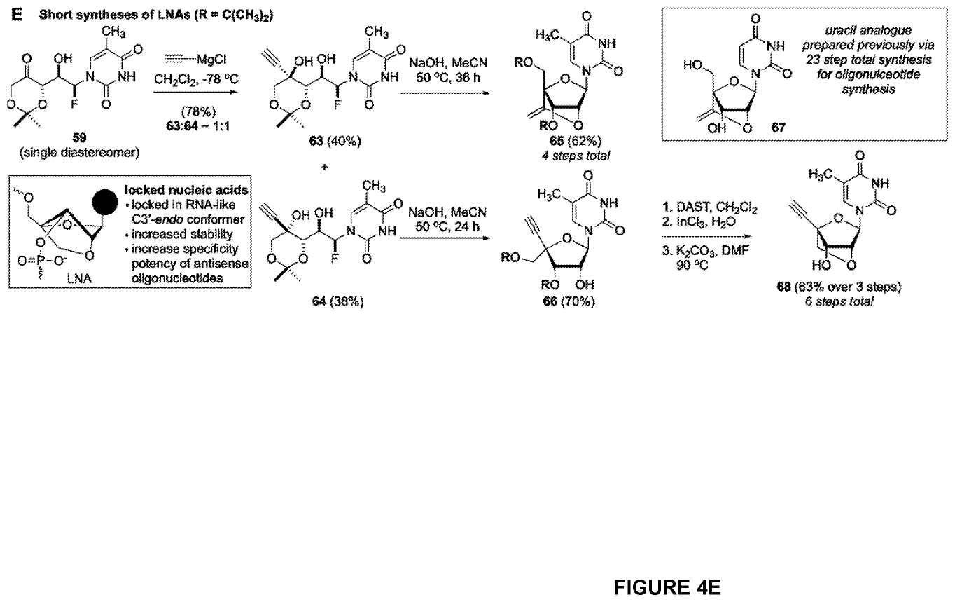

We investigated whether addition of organometallic reagents (rather than reduction with hydride) to a range of αFAR products would provide tertiary alcohols whose subsequent AFD would lead directly to C4′-modified NAs. Toward this goal, we examined reactions of the deazaadenine-substituted fluorohydrin 41 with a range of organometallic reagents (e.g., MeMgCl, MeMgBr, Me₂Zn, Me₃ZnLi, MeLi, Me₂Mg, Me₃MgLi) in CH₂Cl₂ or THF at −78° C., 0° C. or room temperature (FIG. 4A, inset). From this panel, Grignard (e.g., MeMgX) reagents in CH₂Cl₂ proved compatible with the densely functionalized fluorohydrin. The 1,2-addition reaction was performed at −78° C., as higher temperatures promoted 1,2-hydride shift/fluoride displacement as a major degradative pathway. With regards to stereochemistry, the 1,2-addition reactions gave mixtures of tertiary alcohols with a preference for addition from the least hindered face of the carbonyl function in 33 (the re face).(30) When the reaction was executed in CH₂Cl₂ and the crude reaction mixture was allowed to warm to room temperature overnight, the intermediate magnesium alkoxide 42a underwent AFD to provide the C4-modified NA 43 directly. Accordingly, this sequence enables access to enantimoerically enriched C4′-modified NAs in only 3 steps from simple achiral heterocycles and bromoacetaldehyde diethyl acetal. Alternatively, quenching the mixture of magnesium alkoxides 42a and 42b with ammonium chloride followed by a subsequent Lewis acid promoted AFD using InCl₃ gave the anomeric α-D NA 36. Thus, in this case, each of the magnesium alkoxides 42a and 42b cyclize selectively using complimentary base- or Lewis acid promoted AFD processes to afford access to α-L and α-D configured NAs.

We also examined the reaction of several additional organomagnesium reagents with fluorohydrin aldol adducts containing triazole, deazaadenine, thymine, pyrazole or trifluoromethyluracil functions (FIG. 4A). In this study, we found the degree of stereoselectivity in 1,2-addition reactions depended on both the solvent and heterocycle. For example, the addition of MeMgBr to ketofluorohydrins in THF gave mixtures of tertiary alcohols of different composition to those generated in CH₂Cl₂. The addition of MeMgBr to ketofluorohydrins substituted with triazole gave predominantly 1,3-syn-diols that underwent AFD to produce the naturally configured NA α-D-48.

Accordingly, a collection of deazaadenine-substituted NAs 35-39 were readily accessed as both α- and β-anomers. In these studies, base promoted AFD resulted in C3′,C5′-protected NAs (e.g., 49-54), while AFD promoted by Lewis acids resulted in deprotection or protecting group migration (e.g., 44, 47 and 48). As summarized in FIG. 4, a range of densely functionalized C4′-modified NAs could be rapidly accessed from the corresponding ketofluorohydrin aldol adducts, including NAs substituted with methyl, cyclopropyl, aryl and alkynyl groups. Each of the C4′-methyl, cyclopropyl, p-methoxyphenyl, p-chlorophenyl, alkynyl NAs 43-54 were prepared in only 3 or 4 steps total.

Optimization of 1,2-addition reactions is shown in Table 6.

TABLE 6
Optimization of 1,2-addition reaction

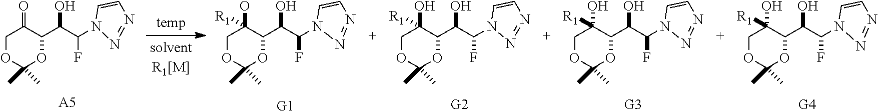

| entry | temp. (° C.) | R₁[M]^{a} | solvent^{b} | G1:G2:G3:G4^{c} | yield^{d} |
|---|---|---|---|---|---|
| 1 | −78 to −10 | MeMgl | CH₂Cl₂ | 5:4.6:1:1 | 84% |
| 2 | −78 to −10 | MeMgBr | CH₂Cl₂ | 5:4:1:1 | 63% |
| 3 | −78 to −10 | MeMgCl | CH₂Cl₂ | 5:4:1:1 | 60% |
| 4 | −78 to −10 | MeMgl | THF | 1:1:1:1 | 92% |
| 5 | 50 | MeMgl | THF | messy | N.D. |
| 6 | −78 to −10 | MeLi | CH₂Cl₂ | messy | N.D. |
| 7 | −78 to −10 | PhLi | CH₂Cl₂ | messy | N.D. |
| 8 | −78 to −10 | Me₃MgLi | CH₂Cl₂ | 4:4:1:1 | 46% |
| 9 | rt | Me₃ZnLi | CH₂Cl₂ | — | 0% |

^{a}3 equiv.
^{b}0.10M.
^{c}determined by analysis of crude reaction mixtures by ¹H NMR.
^{d}isolated yields.

Large Scale αFAR for the Synthesis of Uprifosbuvir.

We examined the synthesis of the D-uridine derivative 56 starting with 900 g of uracil. Without any additional optimization, we were able to generate ˜380 g of the aldol adduct 55 (FIG. 2B), which could be converted into the protected uridine 56 in excellent yield by base-promoted AFD. Oxidation of the C2′—OH function followed by deprotection and addition of MeMgBr in THF gave the tertiary alcohol 57. This later compound is a previously-reported intermediate in the large-scale production of MK-3682 (Uprifosbuvir: 58)(38).

Synthesis of Iminonucleosides, Deoxynucleosides and Locked Nucleic Acids.

We also assessed the utility of this process for accessing an unusual class of NAs known as iminonucleosides or 4′-azanucleosides, whereby the furanose oxygen is replaced by a nitrogen atom. Thus, in one example (FIG. 4C) it was shown that reductive amination of the fluorohydrin aldol adduct 59 (isolated as a single diastereomer as shown) using benzyl amine, followed by a basic work-up led directly to the p-D-configured iminonucleoside 60 in good yield.

To demonstrate the utility of this route for accessing NAs with modifications at both C2′ and C4′, we prepared a C4′-modified, C2′-deoxy NA (FIG. 4D). Here, C4′-allyl thymine 61 was readily prepared in good yield through addition of allylmagnesium bromide to the fluorohydrin aldol adduct 59 followed by base-promoted AFD. A Barton-McCombie deoxygenation then gave the 4′-allyl NA 62 in only 6 steps total from thymine.

To demonstrate utility of this process for NA synthesis, we investigated C4′-functionalization for the preparation of locked nucleic acids (LNAs). Towards a unified LNA synthesis, we evaluated the addition of alkynylmagnesium bromide to the thymine-containing aldol adduct 59 and found the reaction gave two diastereomeric addition products 63 and 64 in excellent overall yield. The major product was transformed directly into the unusual LNA 67 by reacting with NaOH, which promoted both the AFD reaction and a subsequent cyclization between the free alcohol function and alkyne in excellent overall yield. This 4 step total synthesis compares well with the 23-step route reported for the analogous uracil LNA 67 (40). We were also able to generate the unusual alkyne-functionalized LNA 68, a previously unreported scaffold in nucleoside chemistry, by simply effecting an AFD of the 1,2-addition product 64. From here, formation of the 2,2′-anhydrothymidine followed by deprotection and treatment with base in warm DMF(41) gave the LNA 68. This unique scaffold is primed for further diversification through standard click or Sonagashira coupling reactions.

### REFERENCES

1. G. M. Blackburn, Gait, M. J., Loakes, D., Williams, D. M., Ed., Nucleic Acids in Chemistry and Biology, (Royal Society of Chemistry, Cambridge, UK, 2006), pp. 503.
2. C. M. Galmarini, J. R. Mackey, C. Dumontet. Nucleoside Analogues and Nucleobases in Cancer Treatment. Lancet Oncol. 3, 415-424 (2002).
3. E. De Clercq. Highlights in Antiviral Drug Research: Antivirals at the Horizon. Med. Res. Rev. 33, 1215-1248 (2013).
4. L. P. Jordheim, D. Durantel, F. Zoulim, C. Dumontet. Advances in the Development of Nucleoside and Nucleotide Analogues for Cancer and Viral Diseases. Nat. Rev. Drug Discov. 12, 447-464 (2013).
5. D. M. Huryn, M. Okabe. AIDS-Driven Nucleoside Chemistry. Chem. Rev. 92, 1745-1768 (1992).
6. J. Shelton et al. Metabolism, Biochemical Actions, and Chemical Synthesis of Anticancer Nucleosides, Nucleotides, and Base Analogs. Chem. Rev. 116, 14379-14455 (2016).
7. B. Ewald, D. Sampath, W. Plunkett. Nucleoside Analogs: Molecular Mechanisms Signaling Cell Death. Oncogene 27, 6522-6537 (2008).
8. K. L. Seley-Radtke, M. K. Yates. The Evolution of Nucleoside Analogue Antivirals: A Review for Chemists and Non-Chemists. Part 1: Early Structural Modifications to the Nucleoside Scaffold. Antiviral Res. 154, 66-86 (2018).
9. M. K. Yates, K. L. Seley-Radtke. The Evolution of Antiviral Nucleoside Analogues: A Review for Chemists and Non-Chemists. Part II: Complex Modifications to the Nucleoside Scaffold. Antiviral Res. 162, 5-21 (2019).
10. H. Ma et al. Characterization of the Metabolic Activation of Hepatitis C Virus Nucleoside Inhibitor Beta-D-2′-Deoxy-2′-Fluoro-2′-C-Methylcytidine (PSI-6130) and Identification of a Novel Active 5′-Triphosphate Species. J. Biol. Chem. 282, 29812-29820 (2007).
11. E. P. Gillis, K. J. Eastman, M. D. Hill, D. J. Donnelly, N. A. Meanwell. Applications of Fluorine in Medicinal Chemistry. J. Med. Chem. 58, 8315-8359 (2015).
12. J. Deval, M. H. Powdrill, C. M. D'Abramo, L. Cellai, M. Gotte. Pyrophosphorolytic Excision of Nonobligate Chain Terminators by Hepatitis C Virus NS5B Polymerase. Antimicrob. Agents Chemother. 51, 2920-2928 (2007).
13. H. Ohrui. 2′-Deoxy-4′-C-Ethynyl-2-Fluoroadenosine, a Nucleoside Reverse Transcriptase Inhibitor, is Highly Potent Against All Human Immunodeficiency Viruses Type 1 and Has Low Toxicity. Chem. Rec. 6, 133-143 (2006).
14. J. T. Witkowski, R. K. Robins, R. W. Sidwell, L. N. Simon. Design, Synthesis, and Broad Spectrum Antiviral Activity of 1-Beta-D-Ribofuranosyl-1,2,4-Triazole-3-Carboxamide and Related Nucleosides. J. Med. Chem. 15, 1150-1154 (1972).
15. J. Zeidler, D. Baraniak, T. Ostrowski. Bioactive Nucleoside Analogues Possessing Selected Five-Membered Azaheterocyclic Bases. Eur. J. Med. Chem. 97, 409-418 (2015).
16. G. Ni et al. Review of α-Nucleosides: From Discovery, Synthesis to Properties and Potential Applications. RSC Advances 9, 14302-14320 (2019).
17. G. Gumina, G. Y. Song, C. K. Chu. L-Nucleosides as Chemotherapeutic Agents. FEMS Microbiol. Lett. 202, 9-15 (2001).
18. H. Cui et al. Synthesis and Evaluation of alpha-Thymidine Analogues as Novel Antimalarials. J. Med. Chem. 55, 10948-10957 (2012).
19. Chemical Synthesis of Nucleoside Analogues. P. Merino, Ed., (John Wiley & Sons, Inc., 2013), pp. 895.
20. M. Brodszki et al. Synthesis of the Hepatitis B Nucleoside Analogue Lagociclovir Valactate. Org. Process. Res. Dev. 15, 1027-1032 (2011).
21. M. McLaughlin et al. Enantioselective Synthesis of 4′-Ethynyl-2-fluoro-2′-deoxyadenosine (EFdA) via Enzymatic Desymmetrization. Org. Lett. 19, 926-929 (2017).
22. W. T. Markiewicz, M. Wiewiórowski. A New Type of Silyl Protecting Groups in Nucleoside Chemistry. Nucleic Acids Res. 5, s185-s190 (1978).
23. K. R. Campos et al. The Importance of Synthetic Chemistry in the Pharmaceutical Industry. Science 363, eaat0805 (2019).
24. M. Peifer, R. Berger, V. W. Shurtleff, J. C. Conrad, D. W. MacMillan. A General and Enantioselective Approach to Pentoses: a Rapid Synthesis of PSI-6130, the Nucleoside Core of Sofosbuvir. J. Am. Chem. Soc. 136, 5900-5903 (2014).
25. M. W. Powner, B. Gerland, J. D. Sutherland. Synthesis of Activated Pyrimidine Ribonucleotides in Prebiotically Plausible Conditions. Nature 459, 239 (2009).
26. J. S. Teichert, F. M. Kruse, O. Trapp. Direct Prebiotic Pathway to DNA Nucleosides. Angew. Chem. Int. Ed. 58, 9944-9947 (2019).
27. D. Chapdelaine et al. A stereoselective approach to nucleosides and 4′-thioanalogues from acyclic precursors. J. Am. Chem. Soc. 131, 17242-17245 (2009).
28. M. Bergeron-Brlek, T. Teoh, R. Britton. A Tandem Organocatalytic alpha-Chlorination-Aldol Reaction that Proceeds with Dynamic Kinetic Resolution: a Powerful Tool for Carbohydrate Synthesis. Org. Lett. 15, 3554-3557 (2013).
29. M. Bergeron-Brlek, M. Meanwell, R. Britton. Direct Synthesis of Imino-C-Nucleoside Analogues and Other Biologically Active Iminosugars. Nat. Commun. 6, 6903 (2015).
30. C. Grondal, D. Enders. A Direct Organocatalytic Entry to Selectively Protected Aldopentoses and Derivatives. Adv. Synth. Catal. 349, 694-702 (2007).
31. F. A. Davis, P. V. N. Kasu, G. Sundarababu, H. Qi. Nonracemic α-Fluoro Aldehydes: Asymmetric Synthesis of 4-Deoxy-4-fluoro-d-arabinopyranose. J. Org. Chem. 62, 7546-7547 (1997).
32. W. J. Middleton, E. M. Bingham. alpha-Fluorination of Carbonyl Compounds with Trifluoromethyl Hypofluorite. J. Am. Chem. Soc. 102, 4845-4846 (1980).
33. R. Britton, B. Kang. alpha-Haloaldehydes: Versatile Building Blocks for Natural Product Synthesis. Nat. Prod. Rep. 30, 227-236 (2013).
34. D. D. Steiner, N. Mase, C. F. Barbas III. Direct Asymmetric α-Fluorination of Aldehydes. Angew. Chem. Int. Ed. 44, 3706-3710 (2005).
35. E. M. Sánchez-Fernández et al. sp2-Iminosugar O-, S-, and N-Glycosides as Conformational Mimics of α-Linked Disaccharides; Implications for Glycosidase Inhibition. Chem.: Eur. J. 18, 8527-8539 (2012).
36. W. Huang, P. L. Diaconescu. Aromatic C—F Bond Activation by Rare-Earth-Metal Complexes. Organometallics 36, 89-96 (2017).
37. E. De Clercq. C-Nucleosides To Be Revisited. J. Med. Chem. 59, 2301-2311 (2016).
38. A. M. Hyde, R. Calabria, R. Arvary, X. Wang, A. Klapars. Investigating the Underappreciated Hydrolytic Instability of 1,8-Diazabicyclo[5.4.0]undec-7-ene and Related Unsaturated Nitrogenous Bases. Org. Process Res. Dev. 23, 1860-1871 (2019).
39. M. A. Campbell, J. Wengel. Locked vs. Unlocked Nucleic Acids (LNA vs. UNA): Contrasting Structures Work Towards Common Therapeutic Goals. Chem. Soc. Rev. 40, 5680-5689 (2011).
40. P. P. Seth, E. E. Swayze. (2008). 6-Disubstituted or Unsaturated Bicyclic Nucleic Acid Analogs. U.S. Pat. No. 8,278,283 B2. lonis Pharmaceuticals.
41. T. Yamaguchi, M. Horiba, S. Obika. Synthesis and properties of 2′-O,4′-C-spirocyclopropylene bridged nucleic acid (scpBNA), an analogue of 2′,4′-BNA/LNA bearing a cyclopropane ring. Chem. Commun. 51, 9737-9740 (2015).
42. W. Ren et al. Revealing the mechanism for covalent inhibition of glycoside hydrolases by carbasugars at an atomic level. Nat. Commun. 9, 3243 (2018).
43. A. Quintard, J. Rodriguez. Bicatalyzed Three-Component Stereoselective Decarboxylative Fluoro-Aldolization for the Construction of Elongated Fluorohydrins. ACS Catalysis 7, 5513-5517 (2017).
44. T. C. Britton, M. E. LeTourneau. (1995). Process for Anomerizing Nucleosides. U.S. Pat. No. 5,420,266. Eli Lilly and Company.
45. A. M. Downey, C. Richter, R. Pohl, R. Mahrwald, M. Hocek. Direct One-Pot Synthesis of Nucleoside from Unprotected or 5-O-Monoprotected D-Ribose. Org. Lett. 17, 4604-4607 (2015)
46. Z.-Q. Xu, Y.-L. Qui, S. Chokekijchai, H. Mitsuya, J. Zemlicka. Unsaturated Acyclic Analogs of 2′-Deoxyadenosine and Thymidine Containing Fluorine: Synthesis and Biological Activity. J. Med. Chem. 38, 875 (1995)
47. E. Moyroud, E. Biala, P. Strazewski. Synthesis and Enzymatic Digestion of an RNA Nonamer in Both Enantiomeric Forms. Tetrahedron 56, 1475-1484 (2000)
48. A. Hadj-Bouazza, R. Zerrouki, P. Krausz, G. Laumond, A. M. Aubertin, Y. Champavier. New Acyclonucleosides: Synthesis and Anti-HIV Activity. Nucleosides, Nucleotides, and Nucleic Acids 24, 1249-1263, (2005)
49. Y. Mehellou, R. Valente, H. Mottram, E. Walsby, K. I. Mills, J. Balzarini, C. McGuigan. Phosphoramidates of 2′-beta-D-arainouridine (AraU) as Phosphate Prodrugs; Design, Synthesis, in Vitro Activity and Metabolism. Bioorg. Med. Chem. 18, 2439-2446, (2010).
50. A. F. Cook, J. G. Moffatt. Sulfoxide-carbodiimide reactions. VI. Synthesis of 2′- and 3′-ketouridines. J. Am. Chem. Soc. 89, 2697, (1967)
51. S. F. Jenkinson, N. A. Jones, A. Moussa, A. J. Stewart, J. Heinz, G. W. J. Fleet. Anomeric stereospecific synthesis of 2′-C-methyl p-nucleosides; the Holy reaction of cyanamide with 2-C-methyl-D-arabinose. Tetrahedron Letters 48, 4441-4444, (2007)
52. Schrödinger Release 2018-3: MacroModel, Schrödinger, LLC, New York, N.Y. (2019).
53. Gaussian 16, Revision C.01, M. J. Frisch et al. Gaussian, Inc., Wallingford Conn. (2016).
54. P. R. Rablen, S. A. Pearlman, J. Finkbiner. A Comparison of Density Functional Methods for the Estimation of Proton Chemical Shifts with Chemical Accuracy. J. Phys. Chem. A 103, 7357-7363 (1999)
55. M. W. Lodewyk, M. R. Siebert, D. J. Tantillo. Computational Prediction of ¹H and ¹³C Chemical Shifts: A Useful Tool for Natural Product, Mechanistic, and Synthetic Organic Chemistry. Chem. Rev. 112, 1839-1862 (2012)
56. Bruker, APEX3, SAINT and SADABS, Bruker AXS Inc., Madison, Wis. (2016).
57. G. M. Sheldrick. Crystal structure refinement with SHELXL. Acta Crystallogr. Sect. C Struct. Chem. 71, 3-8 (2015).
58. C. B. Hübschle, G. M. Sheldrick, B. Dittrich. ShelXIe: a Qt graphical user interface for SHELXL. J. Appl. Crystallogr. 44, 1281-1284 (2011).
59. C. F. Macrae, I. J. Bruno, J. A. Chisholm, P. R. Edgington, P. McCabe, E. Pidcock, L. Rodriguez-Monge, R. Taylor, J. van de Streek and P. A. Wood. Mercury CSD 2.0—new features for the visualization and investigation of crystal structures. J. Appl. Cryst. 41, 466-470 (2008)
60. T. D. Fenn, D. Ringe, G. A. Petsko. POVScript+: a program for model and data visualization using persistence of vision ray-tracing. J. Appl. Crystallogr. 36, 944-947 (2003).

All citations are hereby incorporated by reference.

The present invention has been described with regard to one or more embodiments. However, it will be apparent to persons skilled in the art that a number of variations and modifications can be made without departing from the scope of the invention as defined in the claims. Therefore, although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to,” and the word “comprises” has a corresponding meaning. It is to be however understood that, where the words “comprising” or “comprises,” or a variation having the same root, are used herein, variation or modification to “consisting” or “consists,” which excludes any element, step, or ingredient not specified, or to “consisting essentially of” or “consists essentially of,” which limits to the specified materials or recited steps together with those that do not materially affect the basic and novel characteristics of the claimed invention, is also contemplated. The elements of the present invention as described may be indicated specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise. Citation of references herein shall not be construed as an admission that such references are prior art to the present invention. All publications are incorporated herein by reference as if each individual publication was specifically and individually indicated to be incorporated by reference herein and as though fully set forth herein. The invention includes all embodiments and variations substantially as hereinbefore described and with reference to the examples and drawings.

1. A method of synthesizing a nucleoside or analogue thereof, the method comprising:
(i) halogenating an aryl- or heteroaryl-substituted acetaldehyde compound by proline catalysis followed by an enantioselective aldol reaction to yield an halohydrin compound;
ii) reducing a halohydrin compound to yield a halohydrin diol compound; and
iii) contacting the halohydrin diol compound with a Lewis acid or a base in an annulative halide displacement (AHD) reaction,
to yield a nucleoside or analogue thereof.

2. The method of claim 1 wherein the Lewis acid is InCl₃ or Sc(OTf)₃.

3. The method of claim 1 wherein the halohydrin diol compound is separated prior to treatment with the Lewis base.

4. The method of claim 1 wherein the base is NaOH.

5. The method of claim 1 wherein the base-AHD reaction yields a C3′,C5′-protected nucleoside or analogue thereof.

6. A method of preparing an intermediate in the synthesis of a nucleoside or analogue thereof, the method comprising:
(i) halogenating a heteroaryl-substituted acetaldehyde compound by proline catalysis followed by an enantioselective aldol reaction to yield an halohydrin compound; and
ii) reducing the halohydrin compound to obtain a halohydrin diol compound, to yield an intermediate in the synthesis of a nucleoside or analogue thereof.

7. The method of claim 6 wherein the intermediate is 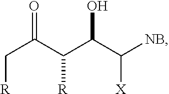
wherein NB is an aryl or heteroaryl, X is a halogen and R is independently —OH, —OC(CH₃)₂O—, —(CH₂)₃—, —CH₂SCH₂—, or —CH₂OCH₂—.

8. The method of claim 6 wherein the intermediate is 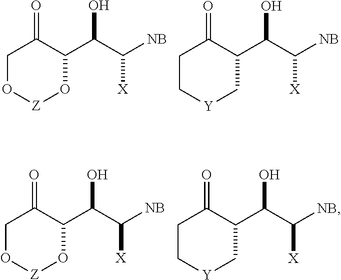
wherein NB is an aryl or heteroaryl, X is a halogen, Y is CH₂, O, S, NR, wherein R is alkyl or aryl, and Z is a protecting group for an alcohol.

9. The method of claim 8 wherein the protecting group for an alcohol is selected from the group consisting of acetonide, silyl protecting group, alkyl protecting group and aryl protecting group.

10. The method of claim 6 wherein the intermediate is 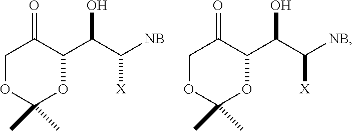
wherein NB is an aryl or heteroaryl and X is a halogen.

11. The method of claim 6 wherein the intermediate is 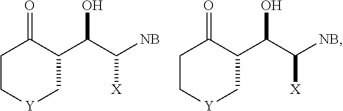
wherein NB is an aryl or heteroaryl, X is a halogen, and Y is CH₂, O, S, NR, wherein R is alkyl or aryl.

12. The method of claim 1 wherein the halohydrin compound is 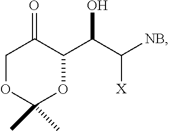
wherein NB is an aryl or heteroaryl and X is a halogen.

13. A method of synthesizing a nucleoside or analogue thereof, the method comprising:
(i) providing a halohydrin diol compound; and
ii) contacting the halohydrin diol compound with a Lewis acid or a base in an annulative halide displacement (AHD) reaction,
to yield a nucleoside or analogue thereof.

14. The method of claim 13 wherein the Lewis acid is InCl₃ or Sc(OTf)₃.

15. The method of claim 13 wherein the halohydrin diol compound is separated prior to treatment with the Lewis base.

16. The method of claim 13 wherein the base is NaOH.

17. The method of claim 13 wherein the base-AHD reaction yields a C3′,C5′-protected nucleoside or analogue thereof.

18. The method of claim 1 wherein the halohydrin diol compound is 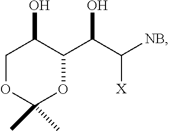
wherein NB is an aryl or heteroaryl and X is a halogen.

19. The method of claim 1 wherein the nucleoside or analogue thereof is a D-nucleoside, a L-nucleoside, a locked nucleic acid, an iminonucleoside, a C4′-modified nucleoside or a C2′-modified nucleoside.

20. The method of claim 1 wherein the nucleoside or analogue thereof is 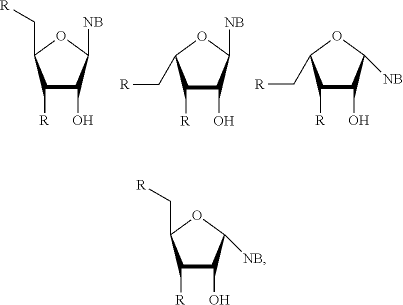
wherein NB is an aryl or heteroaryl and each R is independently —OH, —OC(CH₃)₂O—, —(CH₂)₃—, —CH₂SCH₂—, or —CH₂OCH₂—.
